(19) United States
(12) Patent Application Publication
(10) Pub. No.: US 2024/0368410 A1
(43) Pub. Date: Nov. 7, 2024

(54) SELECTIVE SELF-ASSEMBLED MONOLAYERS VIA SPIN-COATING METHOD FOR USE IN DSA

(71) Applicant: Merck Patent GmbH, Darmstadt (DE)

(72) Inventors: Sachin BOBADE, Somerville, NJ (US); Durairaj BASKARAN, Bridgewater, NJ (US)

(21) Appl. No.: 18/687,109

(22) PCT Filed: Sep. 5, 2022
(86) PCT No.: PCT/EP2022/074549

§ 371 Date: Feb. 27, 2024

Related U.S. Application Data
(60) Provisional application No. 63/241,305, filed on Sep. 7, 2021.

(51) Int. Cl. C09D 5/00; C09D 7/41; C09D 153/00; H01L 21/027

(52) U.S. Cl. CPC C09D 5/002; C09D 7/41; C09D 153/00; H01L 21/0271

(57) ABSTRACT

The present invention relates to a compound of structure (I), wherein A is a core moiety which is selected from structure (Ia), (Ib), (Ic) and (Id), to which is attached through X, a direct valence bond or a divalent linking group, m number of linear alkylene moieties of chain length n where each said linear alkylene moiety has a terminal B reactive moiety, and further wherein * designates possible attachment point of said linear alkylene moieties in each structure, B is selected from —OH, —CH═CH₂, —O—(P═O)(OR)₂, —O—(P═O)(OR)Rₛ, —N₃ and —SH, where n ranges from 8 to 12. The invention also pertains to composition comprising these compounds and the use these compositions to form self-assembled monolayers (SAM) the use of these in DSA processing as neutral or directing layers which can be removed selectively from metal substrates.and the remover solution to accomplish this selective removal.

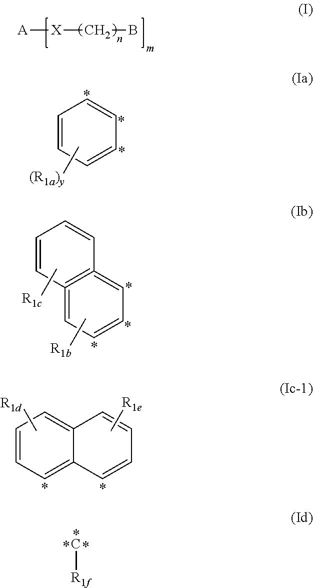

### FIELD OF THE INVENTION

The invention relates to compounds and compositions comprising these compounds which can form self-assembled monolayer on a substrate. These self-assembled monolayers may act as neutral, hydrophobic pinning or hydrophilic pinning layers in directed self-assembly of block copolymer and are useful for fabrication of electronic devices.

### BACKGROUND

Self-assembly of block copolymers is a method useful for generating smaller and smaller patterned features for the manufacture of microelectronic devices in which the critical dimensions (CD) of features on the order of nanoscale can be achieved. Self-assembly methods are desirable for extending the resolution capabilities of microlithographic technology for repeating features such as an array of contact holes or posts. In a conventional lithography approach, ultraviolet (UV) radiation may be used to expose through a mask onto a photoresist layer coated on a substrate or layered substrate. Positive or negative photoresists are useful, and these can also contain a refractory element such as silicon to enable dry development with conventional integrated circuit (IC) plasma processing. In a positive photoresist, UV radiation transmitted through a mask causes a photochemical reaction in the photoresist such that the exposed regions are removed with a developer solution or by conventional IC plasma processing. Conversely, in negative photoresists, UV radiation transmitted through a mask causes the regions exposed to radiation to become less removable with a developer solution or by conventional IC plasma processing. An integrated circuit feature, such as a gate, via or interconnect, is then etched into the substrate or layered substrate, and the remaining photoresist is removed. When using conventional lithographic exposure processes, the dimensions of features of the integrated circuit feature are limited. Further reduction in pattern dimensions is difficult to achieve with radiation exposure due to limitations related to aberrations, focus, proximity effects, minimum achievable exposure wavelengths and maximum achievable numerical apertures. The need for large-scale integration has led to a continued shrinking of the circuit dimensions and features in the devices. In the past, the final resolution of the features has been dependent upon the wavelength of light used to expose the photoresist, which has its own limitations. Directed (a.k.a. guided) self-assembly (DSA) techniques, such as graphoepitaxy and chemoepitaxy using block copolymer imaging, which employ a patterned area on a substrate, are highly desirable techniques used to enhance resolution while reducing CD variation. These techniques can be employed to either enhance conventional UV lithographic techniques or to enable even higher resolution and CD control in approaches employing EUV, e-beam, deep UV or immersion lithography. The directed self-assembly block copolymer comprises a block of etch resistant copolymeric unit and a block of highly etchable copolymeric unit, which when coated, aligned and etched on a substrate give regions of very high-density patterns.

For directed (guided), or unguided self-assembly, of a block copolymer film, respectively, on a patterned or non-patterned substrate area, typically the self-assembly process of this block polymer layer occurs during annealing of this film overlying a neutral layer. This neutral layer over a semiconductor substrate may be an unpatterned neutral layer, or in chemoepitaxy or graphoepitaxy, this neutral layer may contain, respectively, graphoepitaxy or chemoepitaxy guiding features (formed through the above-described UV lithographic technique). During annealing of the block copolymer film, the underlying, neutral layer, directs the nano-phase separation of the block copolymer domains. One example is the formation phase separated domains which are lamellas or cylinders perpendicular to the underlying neutral layer surface. These nano-phase separated block copolymer domains, form a pre-pattern (e.g., line and space L/S) which may be transferred into the substrate through an etching process (e.g., plasma etching). In graphoepitaxy, or in chemoepitaxy, these guiding features may dictate both pattern rectification and pattern rectification. In the case of an unpatterned neutral layer this produces a repeating array of for instance L/S or CH. For example, in a conventional block copolymer such as poly(styrene-b-methyl methacrylate (P(S-b-MMA)), in which both blocks have similar surface energies at the BCP-air interface, this can be achieved by coating and thermally annealing the block copolymer on a layer of non-preferential or neutral material that is grafted or cross-linked at the polymer-substrate interface.

In the graphoepitaxy directed self-assembly method, the block copolymers self organizes around a substrate that is pre-patterned with conventional lithography (Ultraviolet, Deep UV, e-beam, Extreme UV (EUV) exposure source) to form repeating topographical features such as a line/space (L/S) or contact hole (CH) pattern. In an example of a L/S directed self-assembly array, the block copolymer can form self-aligned lamellar regions which can form parallel line-space patterns of different pitches in the trenches between pre-patterned lines, thus enhancing pattern resolution by subdividing the space in the trench between the topographical lines into finer patterns. For example, a diblock copolymer or a triblock copolymer which is capable of microphase separation and comprises a block rich in carbon (such as styrene or containing some other element like Si, Ge, Ti) which is resistant to plasma etch, and a block which is highly plasma etchable or removable, can provide a high-resolution pattern definition. Examples of highly etchable blocks can comprise monomers which are rich in oxygen and which do not contain refractory elements and are capable of forming blocks which are highly etchable, such as methyl methacrylate. The plasma etching gases used in the etching process of defining the self-assembly pattern typically are those used in processes employed to make integrated circuits (IC). In this manner, very fine patterns can be created in typical IC substrates than were definable by conventional lithographic techniques, thus achieving pattern multiplication. Similarly, features such as contact holes can be made denser by using graphoepitaxy in which a suitable block copolymer arranges itself by directed self-assembly around an array of contact holes or posts defined by conventional lithography, thus forming a denser array of regions of etchable and etch resistant domains which when etched give rise to a denser array of contact holes. Consequently, graphoepitaxy has the potential to offer both pattern rectification and pattern multiplication.

In chemical epitaxy, or pinning chemical epitaxy, the self-assembly of the block copolymer is formed on a surface whose guiding features are regions of differing chemical affinity, having no, or insignificant topography (a.k.a. non-guiding topography) which predicates the directed self-assembly process. For example, the surface of a substrate could be patterned with conventional lithography (e.g., UV, Deep UV, e-beam EUV) to create surfaces of different chemical affinity in a line and space (L/S) pattern in which exposed areas whose surface chemistry had been modified by irradiation alternate with areas which are unexposed and show no chemical change. These areas present no topographical difference but do present a surface chemical difference or pinning to direct self-assembly of block copolymer segments. Specifically, the directed self-assembly of a block copolymer whose block segments contain etch resistant (such as styrene repeat unit) and rapidly etching repeat units (e.g., methyl methacrylate repeat units) would allow precise placement of etch resistant block segments and highly etchable block segments over the pattern. This technique allows for the precise placement of these block copolymers and the subsequent pattern transfer of the pattern into a substrate after plasma or wet etch processing. Chemical epitaxy has the advantage that it can be fined tuned by changes in the chemical differences to help improve line-edge roughness and CD control, thus allowing for pattern rectification. Other types of patterns such as repeating contact holes (CH) arrays could also be pattern rectified using chemoepitaxy.

These neutral layers are layers on a substrate or the surface of a treated substrate which have no affinity for either of the block segment of a block copolymer employed in directed self-assembly. In the graphoepitaxy method of directed self-assembly of block copolymer, neutral layers are useful as they allow the proper placement or orientation of block polymer segments for directed self-assembly which leads to proper placement of etch resistant block polymer segments and highly etchable block polymer segments relative to the substrate. For instance, in surfaces containing line and space features which have been defined by conventional radiation lithography, a neutral layer allows block segments to be oriented so that the block segments are oriented perpendicular to the surface of the substrates, an orientation which is ideal for both pattern rectification and pattern multiplication depending on the length of the block segments in the block copolymer as related to the length between the lines defined by conventional lithography. If a substrate interacts too strongly with one of the block segments it would cause it to lie flat on that surface to maximize the surface of contact between the segment and the substrate; such a surface would perturb the desirable perpendicular alignment which can be used to either achieve pattern rectification or pattern multiplication based on features created through conventional lithography. Modification of selected small areas or pinning of substrate to make them strongly interactive with one block of the block copolymer and leaving the remainder of the surface coated with the neutral layer can be useful for forcing the alignment of the domains of the block copolymer in a desired direction, and this is the basis for the pinned chemoepitaxy or graphoepitaxy employed for pattern multiplication. The pinning area may be one which is hydrophilic having a greater affinity for example to polar block copolymer segments such as the polymethyl methacrylate block segment in a block copolymer of styrene and methyl methacrylate or alternatively be a pinning area which may be hydrophobic having a greater affinity for example to the polystyrene block segments in a block copolymer of styrene and methyl methacrylate.

Area selective deposition of organic or inorganic material is an important process in IC industry which requires selectivity exclusive to either metal or dielectric via direct and indirect assembly processes. One such application is passivation of dielectric or metal surface on a given pattern substrates for area selective deposition of metal oxide via atomic layer deposition. This application requires selective grafting of organic materials such as self-assembled monolayers (SAM) or chain end functional polymers (brush) for introducing subsequent deposition or assembly process that enables passivation of selective area underneath. An easy method such as spin-coating organic materials that can exhibit selectivity to specific area of in chip making lithographic processes is highly sought after in industry for ALD or DSA. Common methods of SAM deposition are solution or immersion method and vapor phase. Both these methods have its own merits and demerits. Chemistry and processing of SAM precursors limits its selective deposition via spin-coat method.

There is a need for a novel materials which can easily form self-assembled monolayers (SAM) with a high density of moieties which can impart neutral or pinning characteristics on semiconductor (e.g., Si, GaAs, and the like), metals (e.g., Cu, W, Mo, Al, Zr, Ti, Hf, Au and the like) and metal oxides (e.g., Copper oxide, Aluminum oxide, Hafnium oxide, Zirconium oxide, Titanium oxide and the like) substrates through a simple spin coating, followed by a post coat bake to affect formation of the SAM. Such self-assembled monolayers would have a much higher density and uniformity of moieties which impart neutral or pinning characteristics than are found in convention polymer brush layers or crosslinked and/or grafted MAT materials. Additionally, MAT materials as DSA directing have considerable thickness compared to SAM materials, which may lead to residue issues during DSA processing, further MAT material may also contain other additives such radical generation, thermal acid generator or other additives which may contaminate the substrates used in DSA. A MAT layer in the present context is a crosslinked layer which is insoluble to any layer coated on top of it, which can be used as a DSA neutral or pinning layer.

There is also a need for such SAM neutral layers which can easily be selectively cleaved from a metal substrate which further enhance the lithographic performance of the directed self-assembly materials and processes by reducing the number of processing steps and providing better pattern resolution with good lithographic performance.

### DETAILED DESCRIPTION OF DRAWINGS

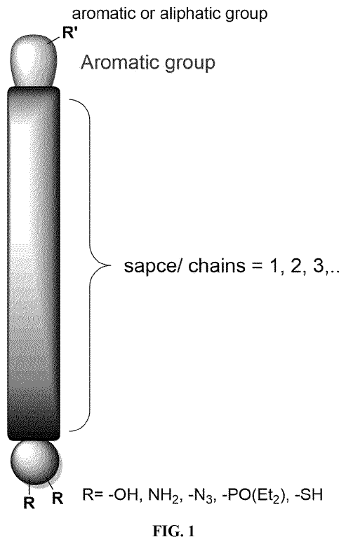

FIG. 1: Schematic structure of spin-coat able SAM.

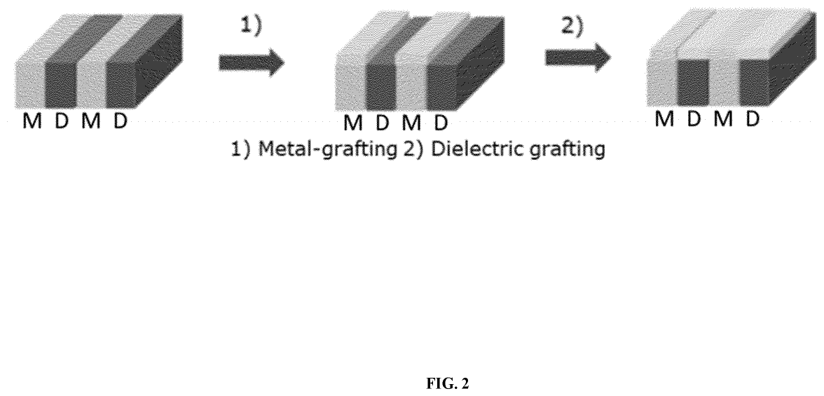

FIG. 2: Direct flow for chemically modified prepattern

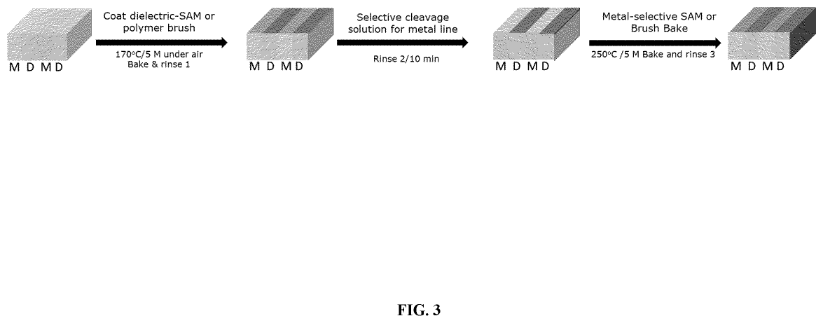

FIG. 3: Dielectric first and metal second using modified flow of selective cleavage of organic moieties from metal substrates using remover solutions

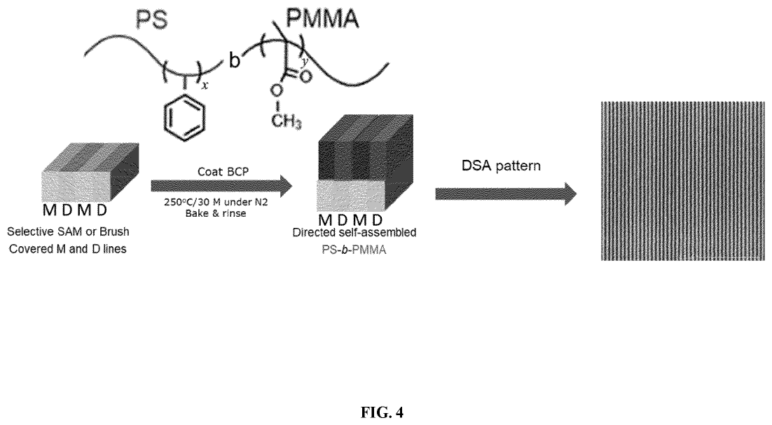

FIG. 4: Directed self-assembly of block-copolymer (PS-b-PMMA) on a chemical prepattern modified with SAM's or polymer brush with selective pinning strength towards PS vs PMMA

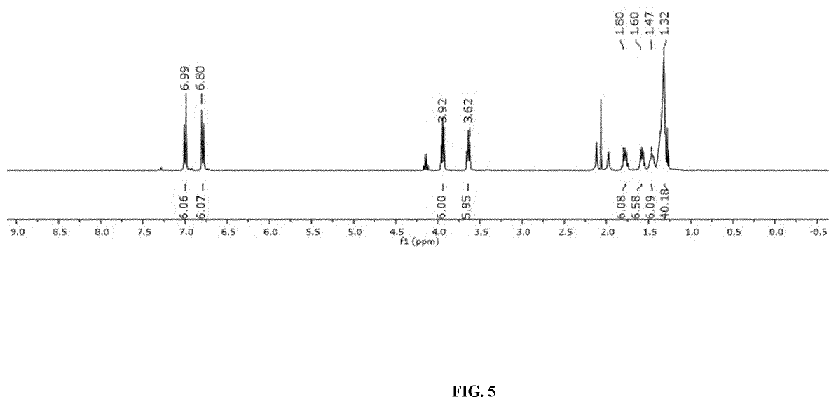

FIG. 5: ¹H NMR of M-1

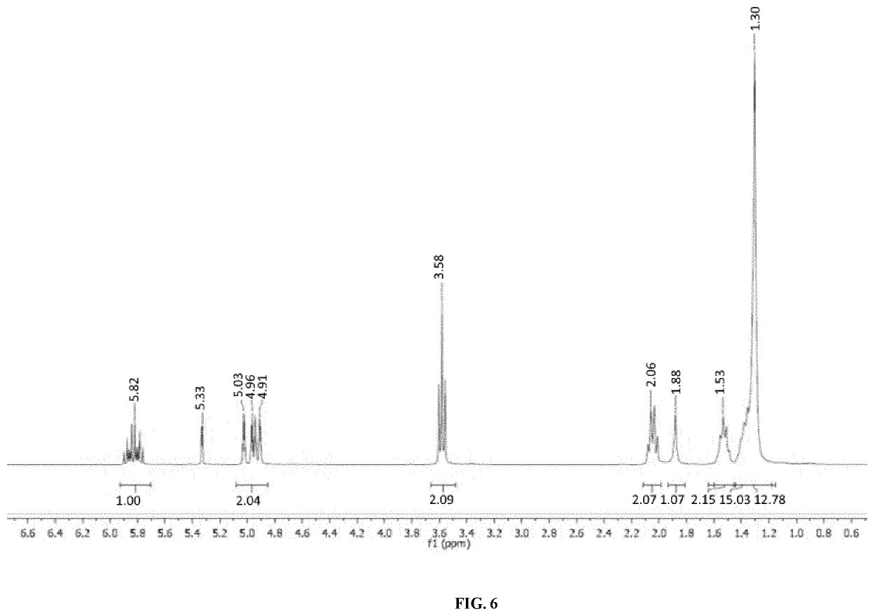

FIG. 6 ¹H NMR spectrum of undecenyl mesylate

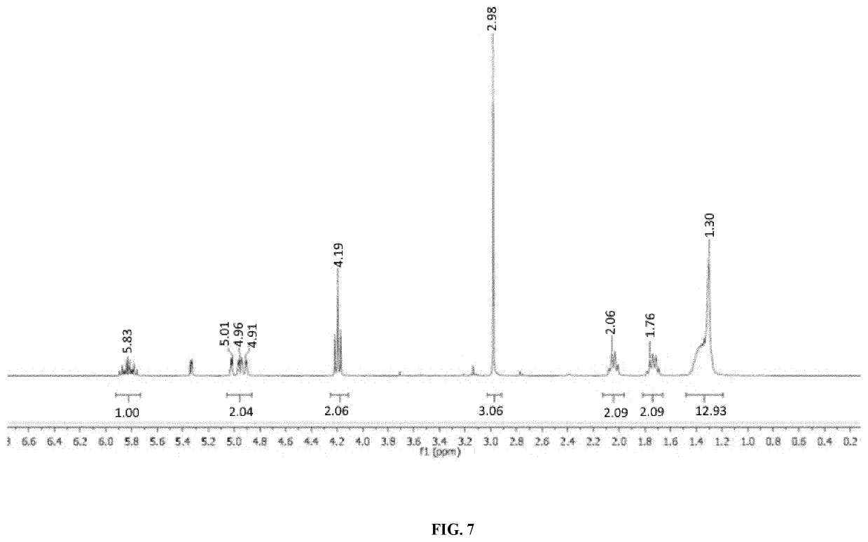

FIG. 7 1H NMR spectrum of undecenyl ether glycerin

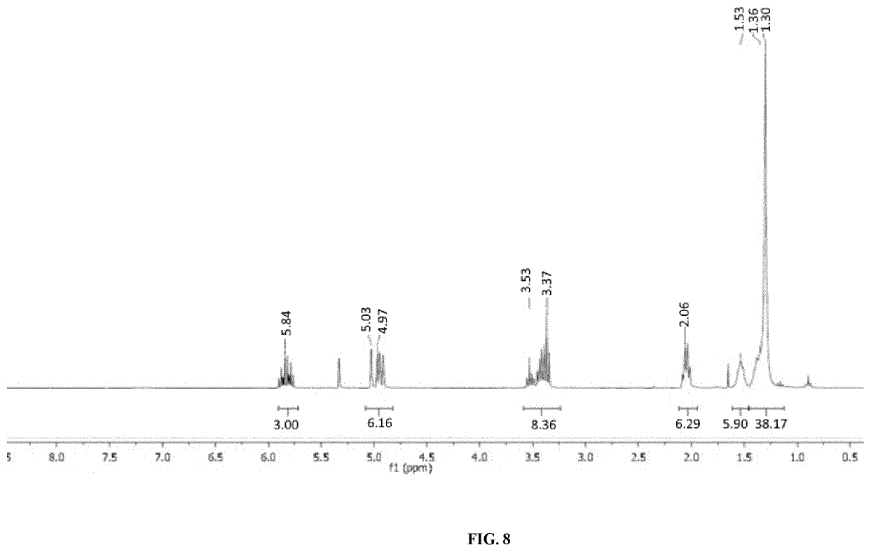

FIG. 8 ¹H NMR spectrum of undecyl tris-ether glycerol

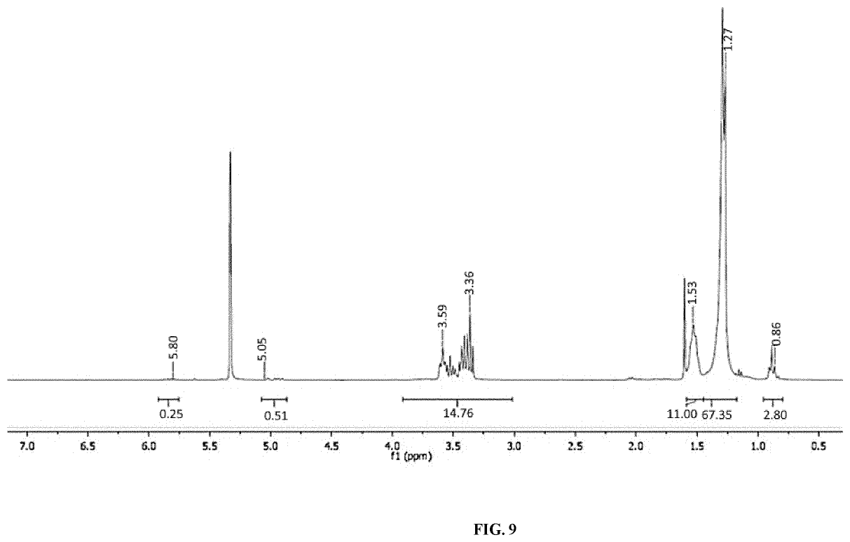

FIG. 9 ¹H NMR spectrum of M-2

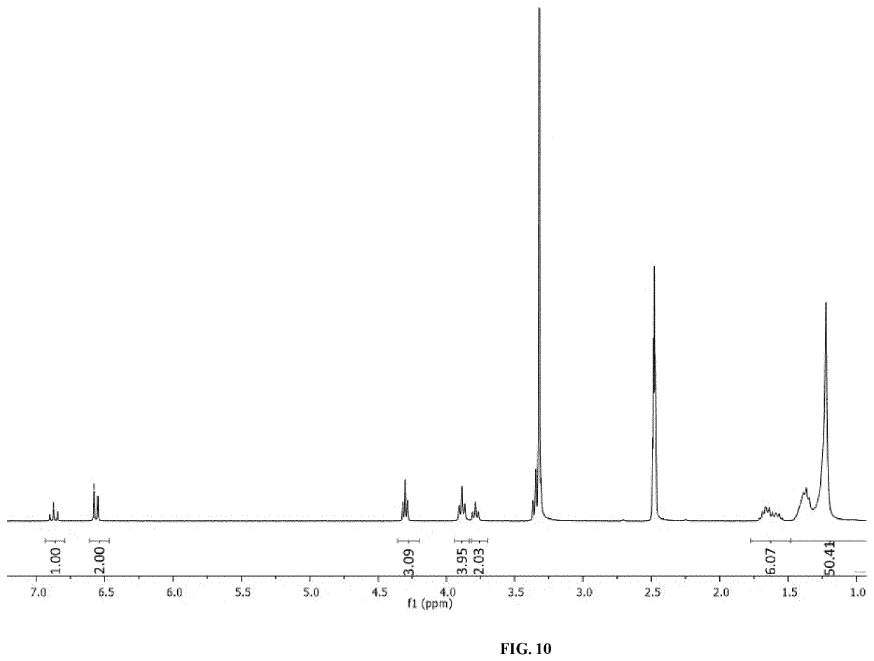

FIG. 10 ¹H NMR spectrum of M-3

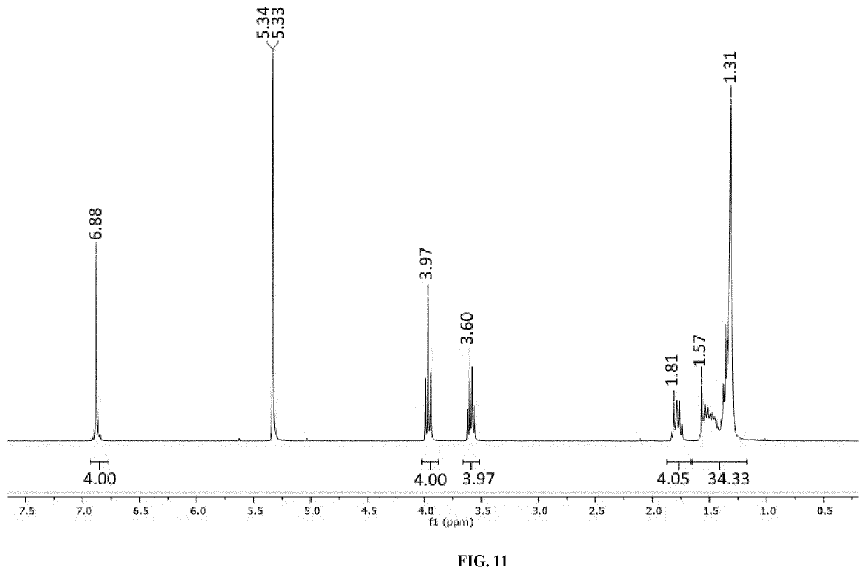

FIG. 11 ¹H NMR spectrum of M-4

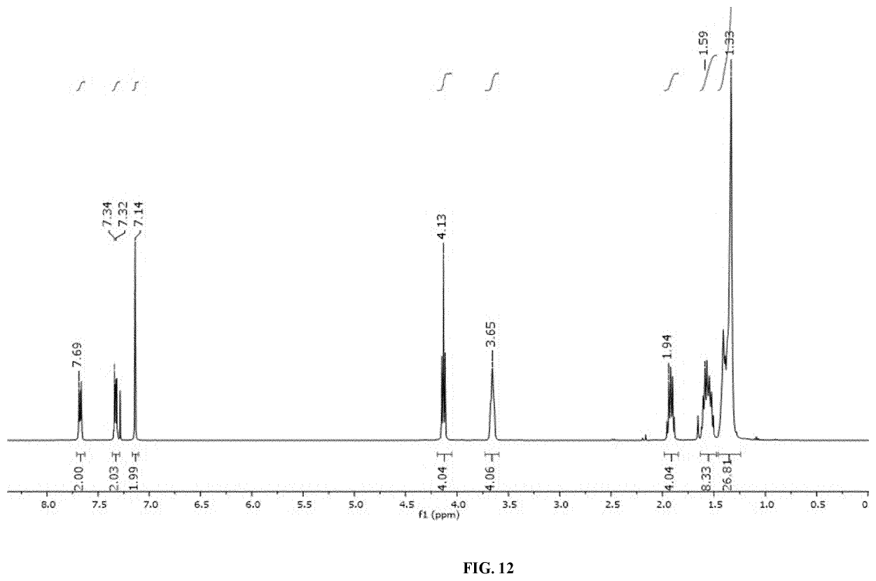

FIG. 12 ¹H NMR spectrum of M-5

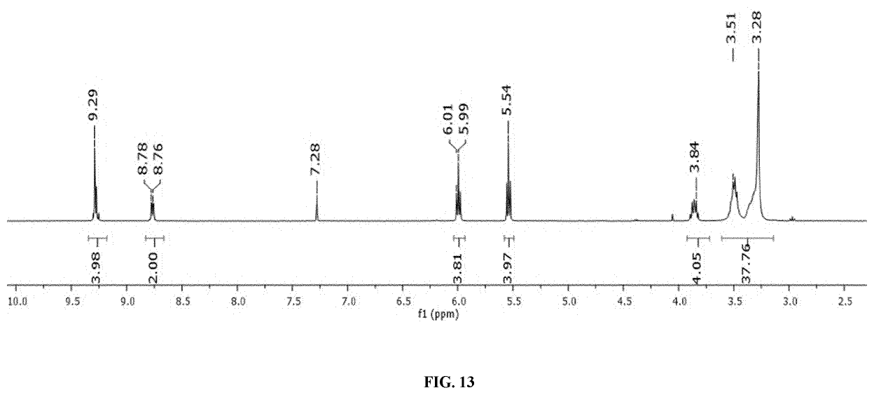

FIG. 13 ¹H NMR spectrum of M-6

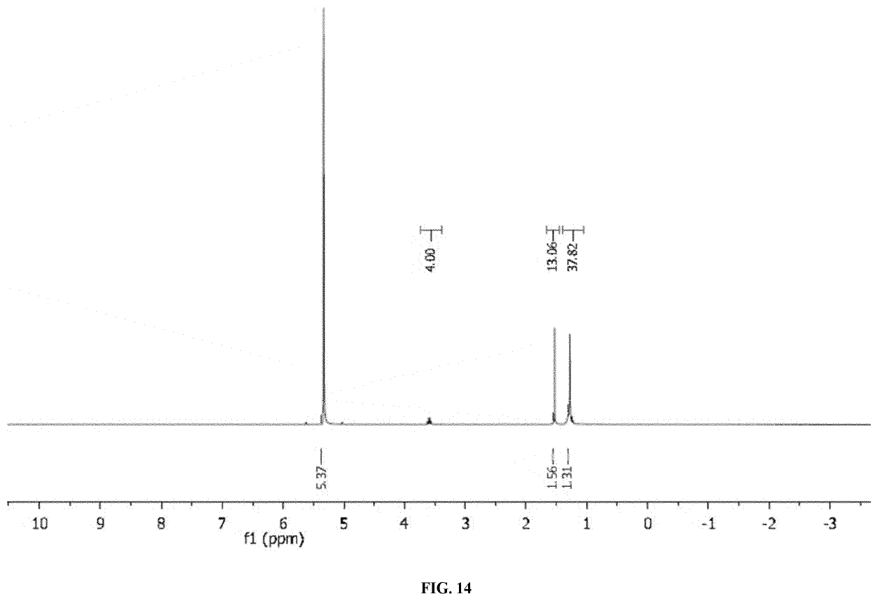

FIG. 14 ¹H NMR spectrum of M-7

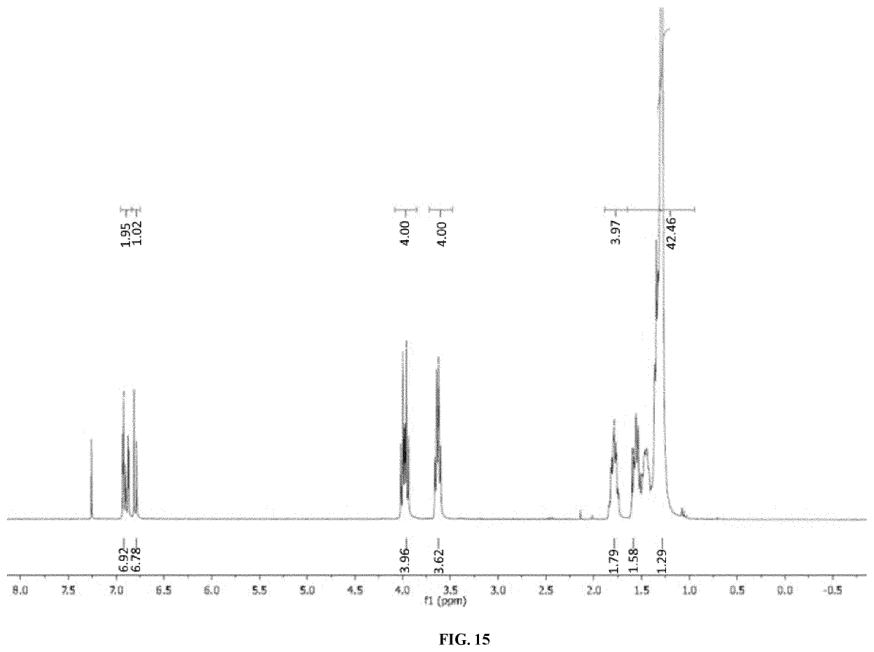

FIG. 15 1H NMR spectrum of M-8

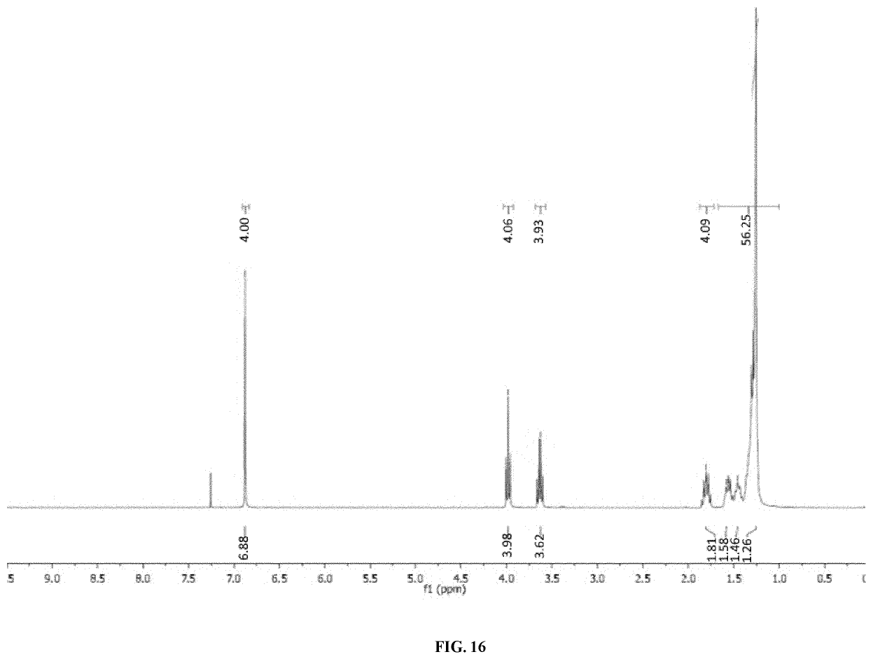

FIG. 16 ¹H NMR spectrum of M-9

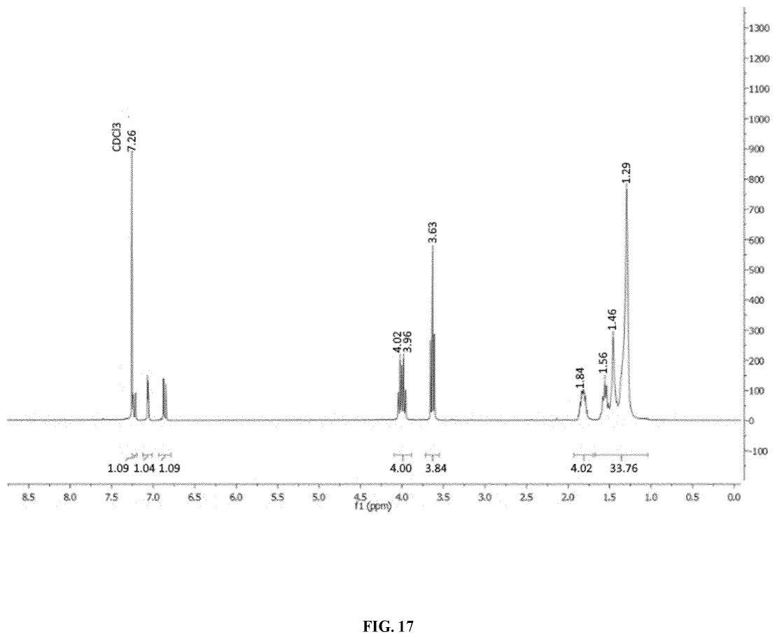

FIG. 17 ¹H NMR spectrum of M-10

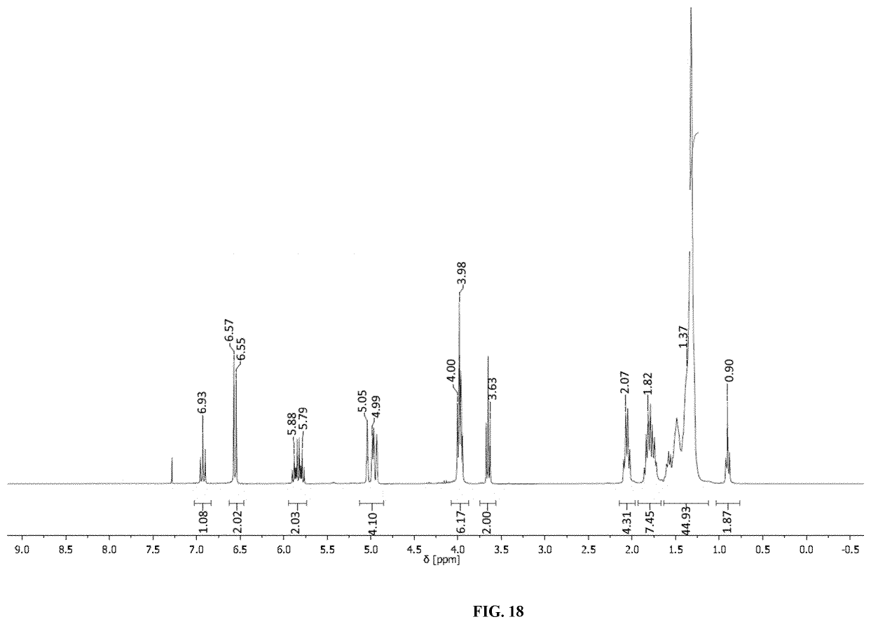

FIG. 18 ¹H NMR spectrum of M-11

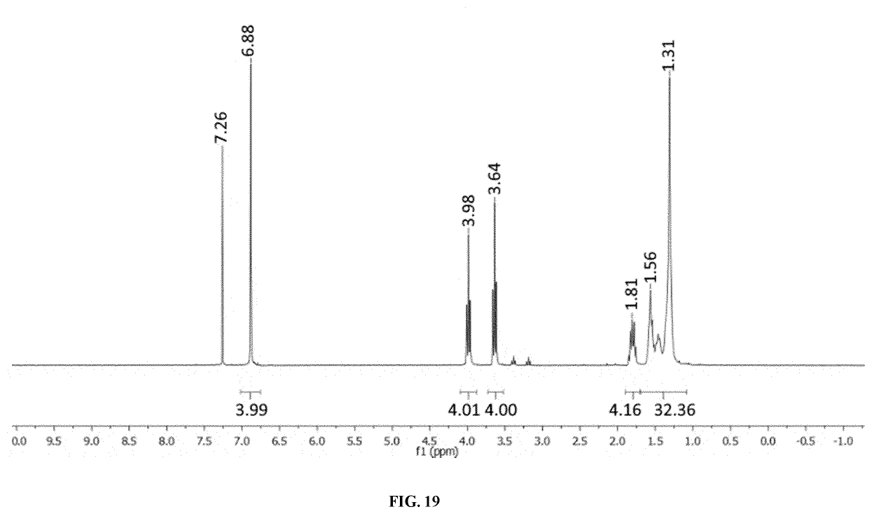

FIG. 19 ¹H NMR spectrum of M-12

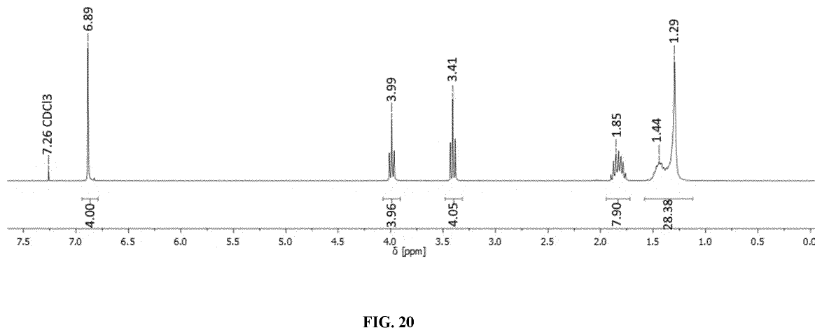

FIG. 20 ¹H NMR spectrum of bromo precursor in Scheme 5

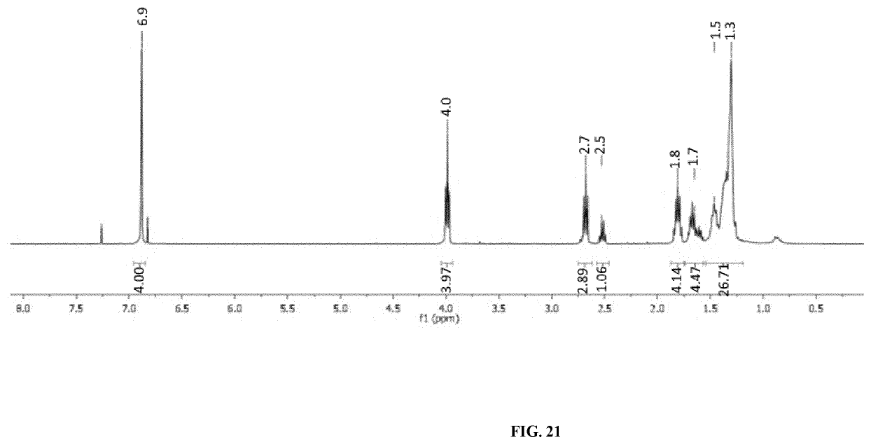

FIG. 21 ¹H NMR spectrum of M-13 (thiol groups)

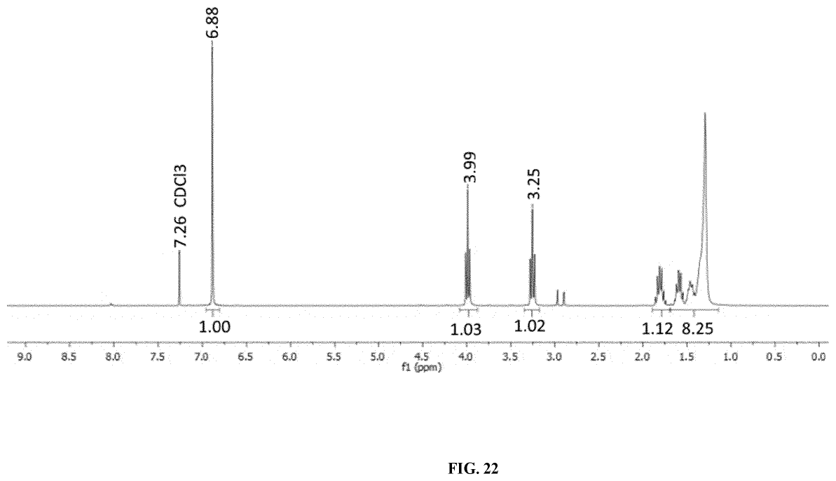

FIG. 22 ¹H NMR spectrum of M-14 (azide group)

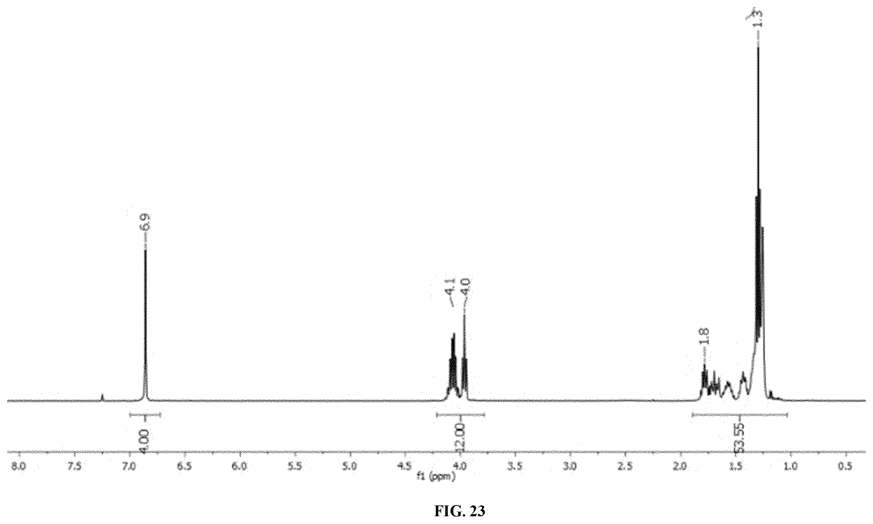

FIG. 23 ¹H NMR spectrum of M-15 (diethyl phosphonate groups)

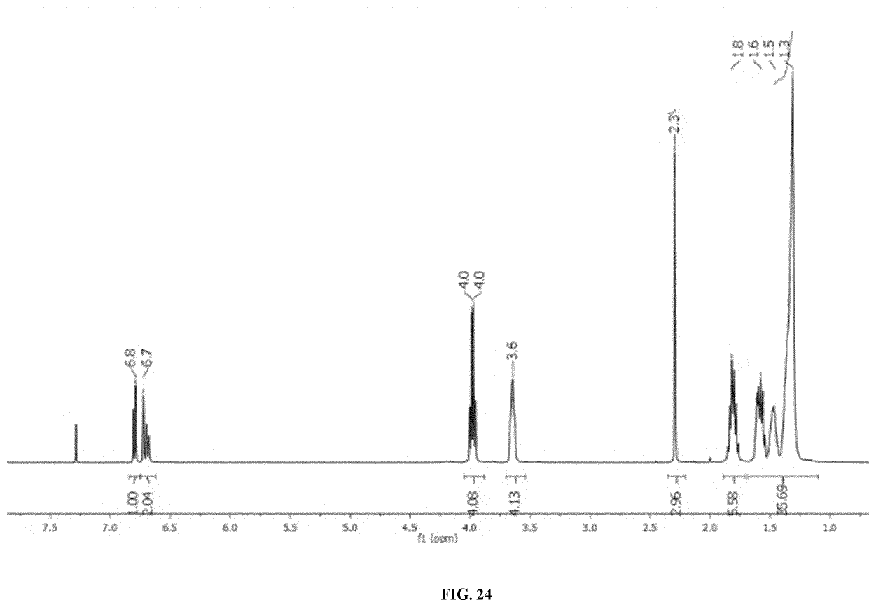

FIG. 24 ¹H NMR spectrum of M-16 with methyl substitution on aromatic ring

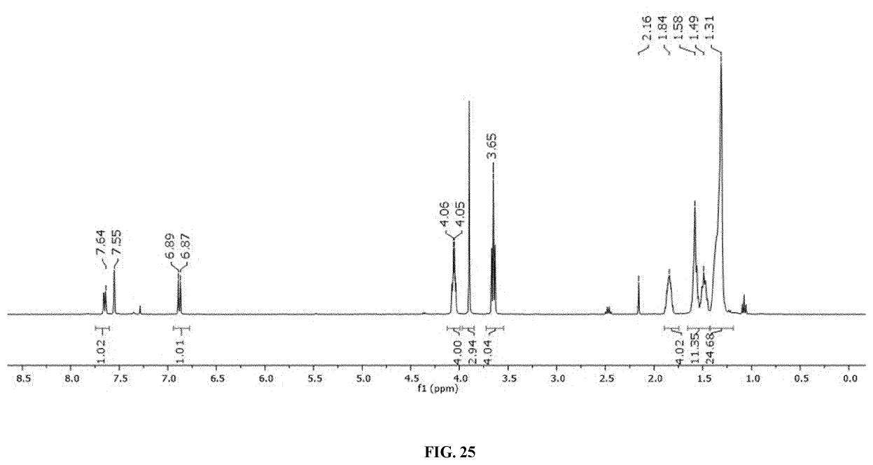

FIG. 25 ¹H NMR spectrum of M-17 with methyl ester substitution on aromatic ring

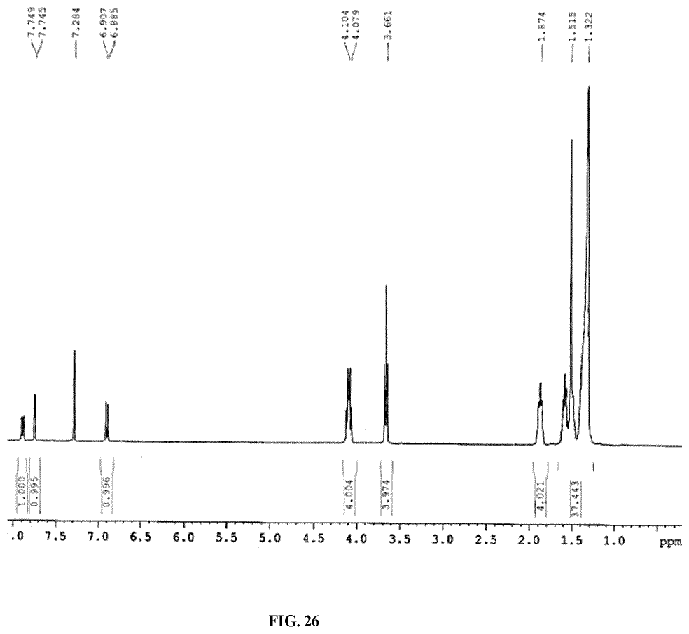

FIG. 26 ¹H NMR spectrum of M-18 with nitro group on aromatic ring

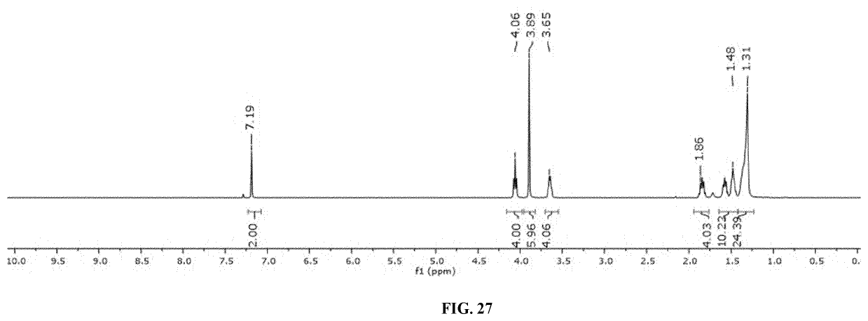

FIG. 27 ¹H NMR spectrum of M-19 with di-methyl ester substitution on aromatic group

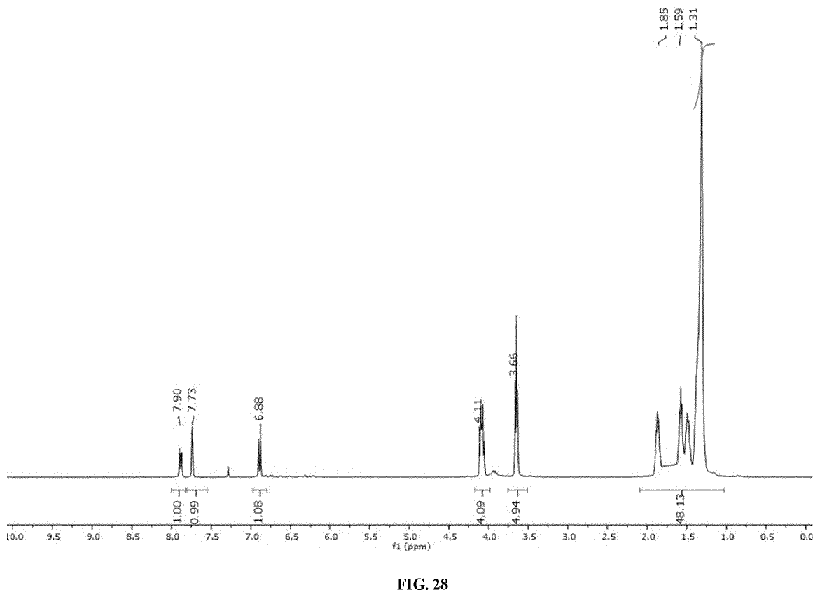

FIG. 28 ¹H NMR spectrum of M-20 with amino group on aromatic ring

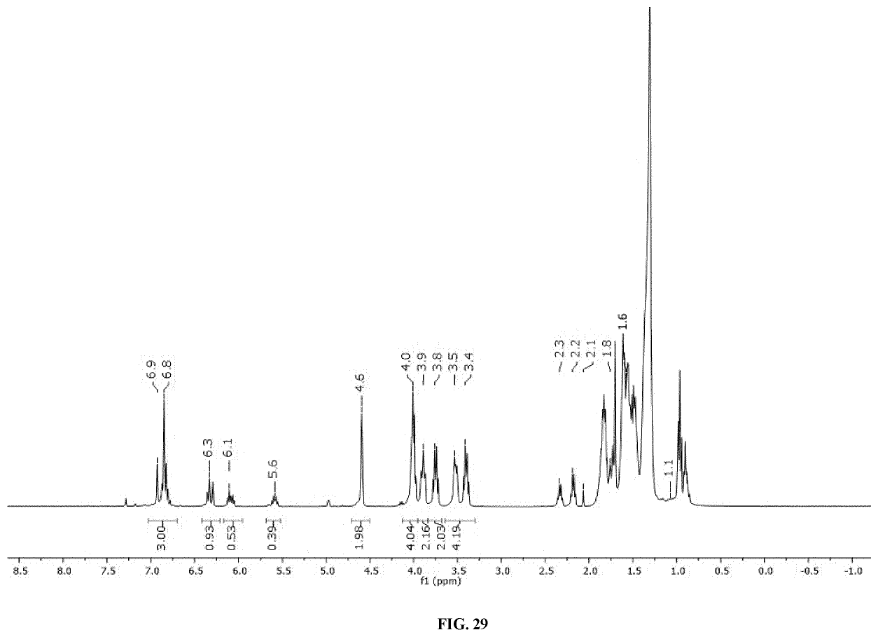

FIG. 29 ¹H NMR Spectrum of THP protected M-21 with 1-hexene substitution on aromatic

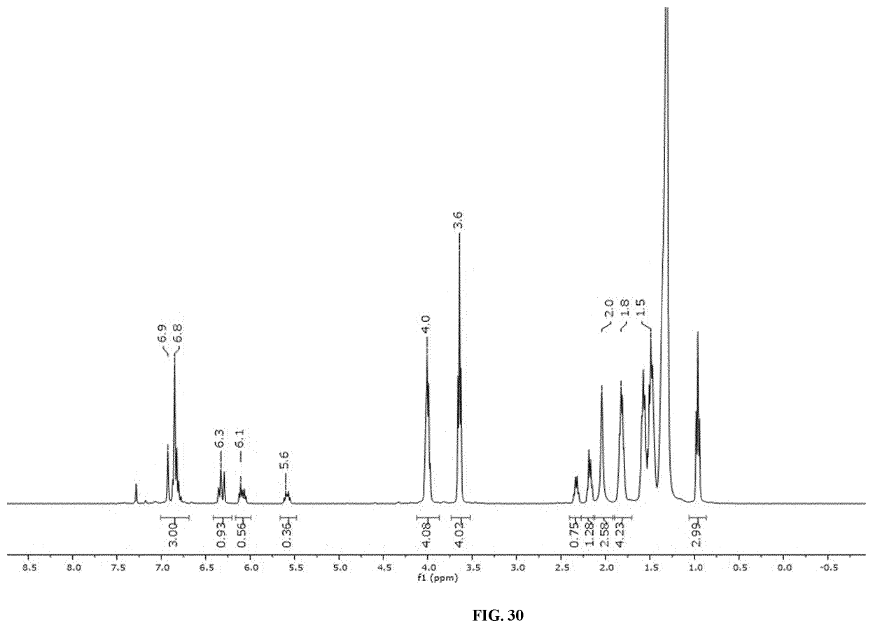

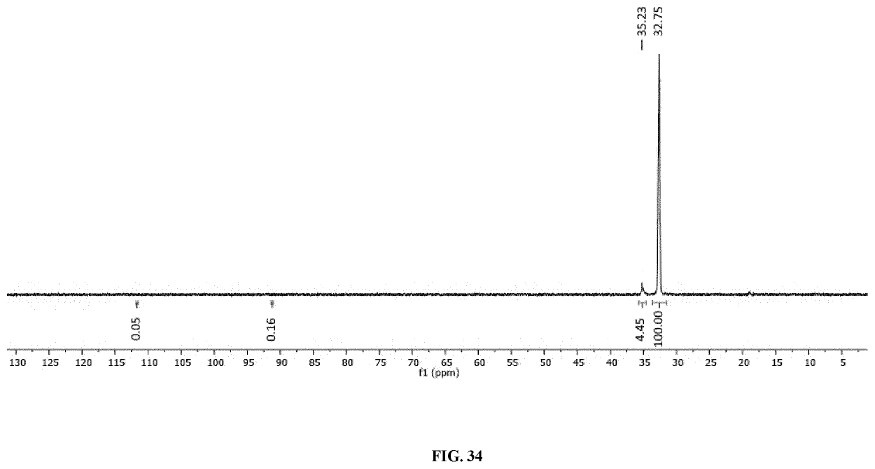

FIG. 30 FIG. 34: ¹H NMR Spectrum of M-21 with 1-hexene substitution on aromatic group

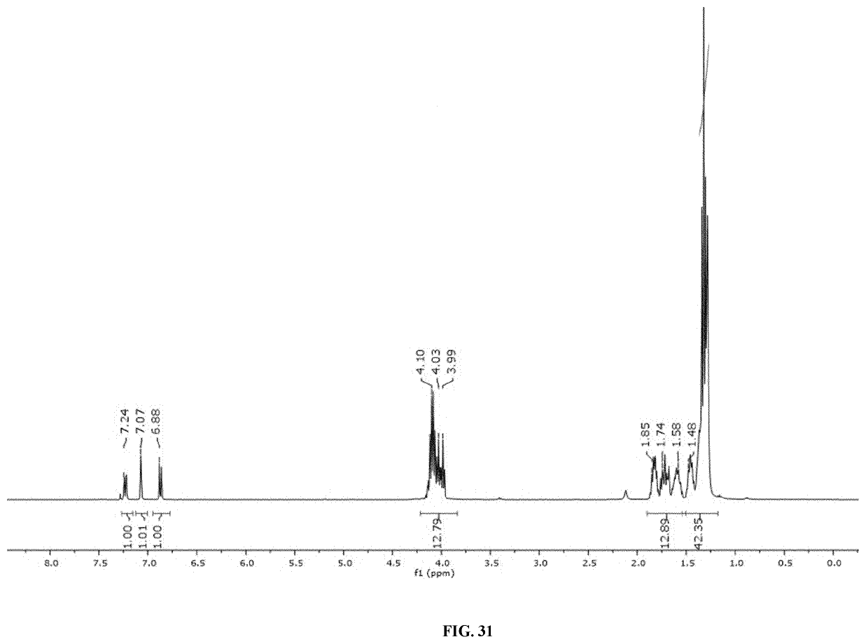

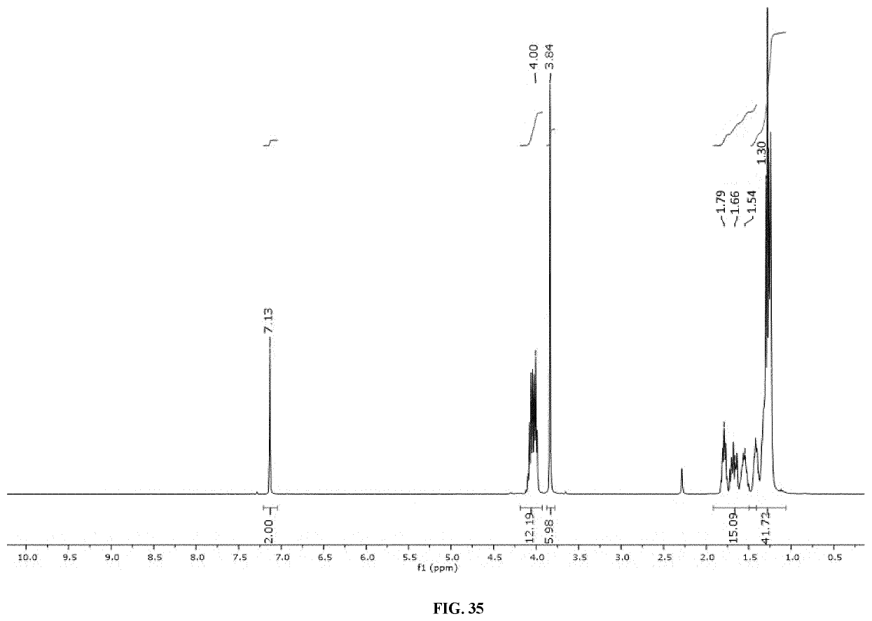

FIG. 31 FIG. 35: 1H NMR spectrum of M-22

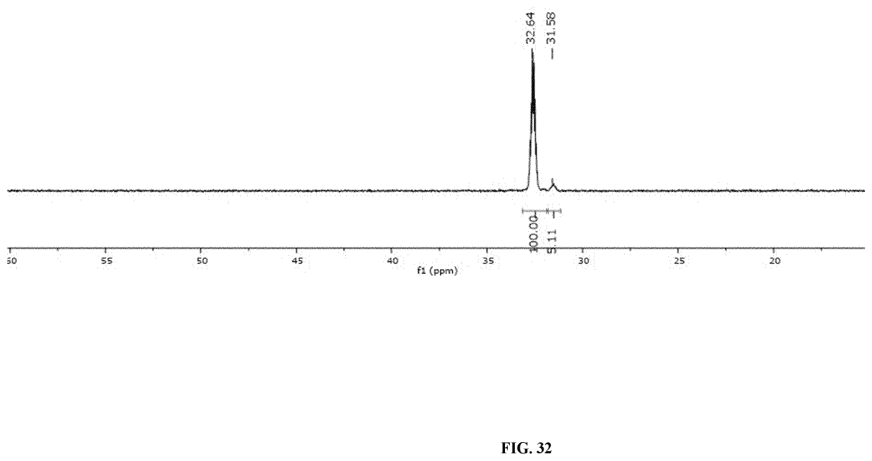

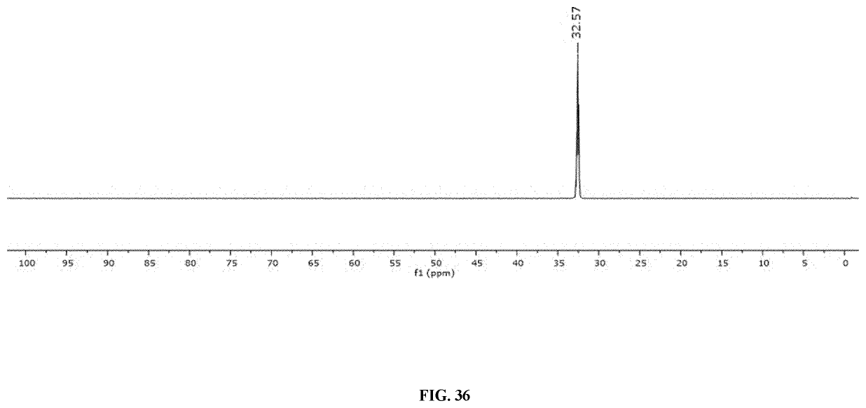

FIG. 32 FIG. 36: ³¹P NMR of M-22

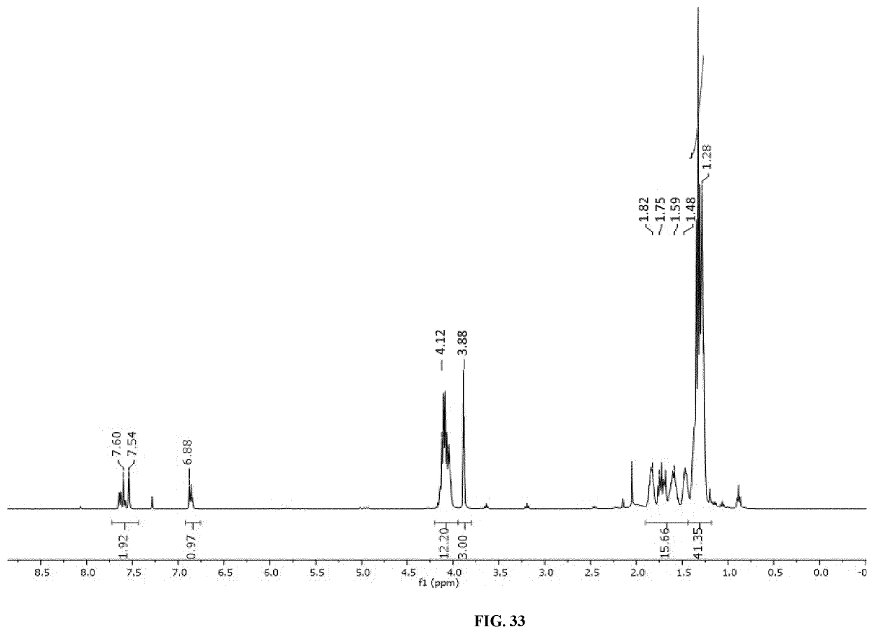

FIG. 33 ¹H NMR spectrum of M-23

FIG. 34 ³¹P NMR spectrum of M-23

FIG. 35 1H NMR spectrum of M-24

FIG. 36 ³¹P NMR spectrum of M-24

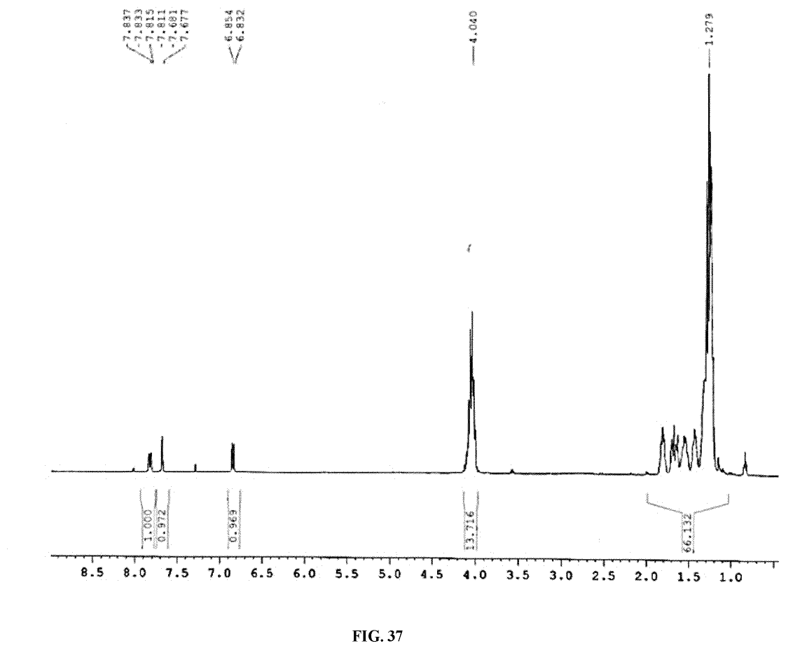

FIG. 37 1H NMR spectrum of M-25

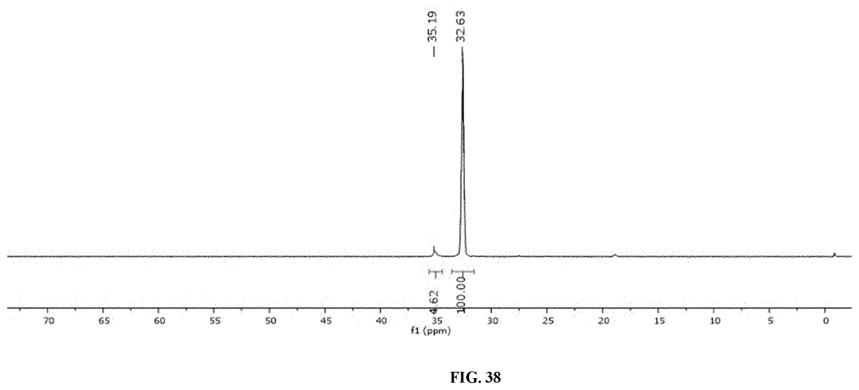

FIG. 38 ³¹P NMR spectrum of M-25

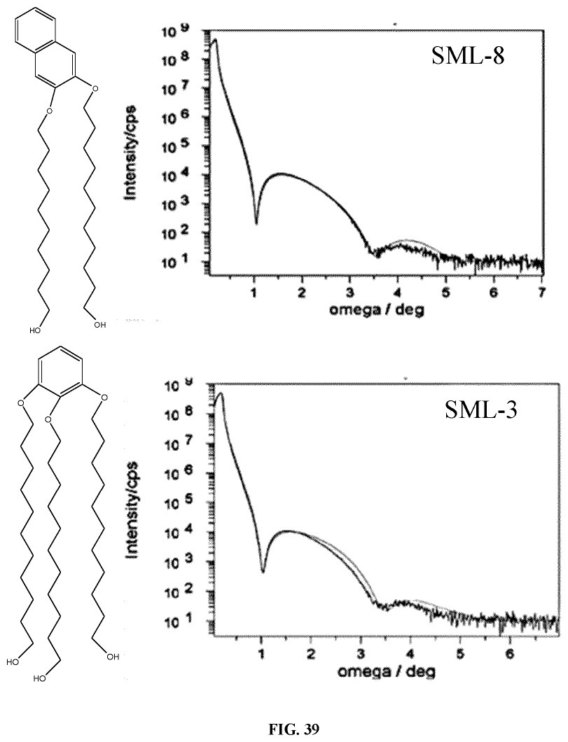

FIG. 39 XRR of SML-8 and SML-3

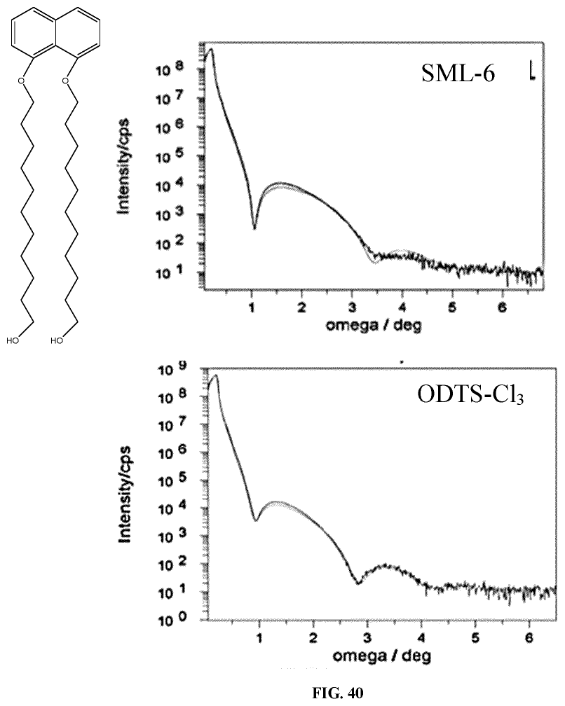

FIG. 40 XRR of SML-6 and comparative material ODTS-Cl₃

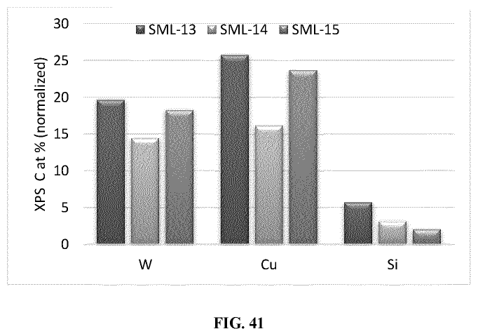

FIG. 41 Selective SAM deposition on metal (SML-13, SML-14, SML-15)

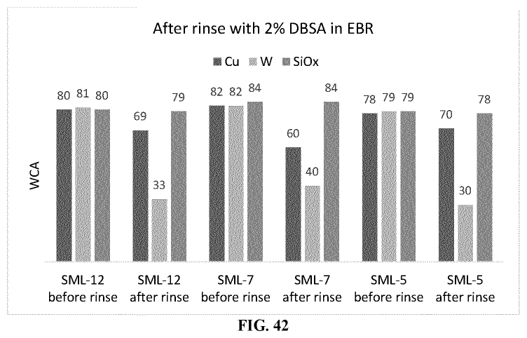

FIG. 42 Selective removal of metal SAM (SML-12, SML-7, SML-5 using dodecylbenzene sulfonic acid (DBSA) solution in mixture of PGME:PGMEA (70:30)

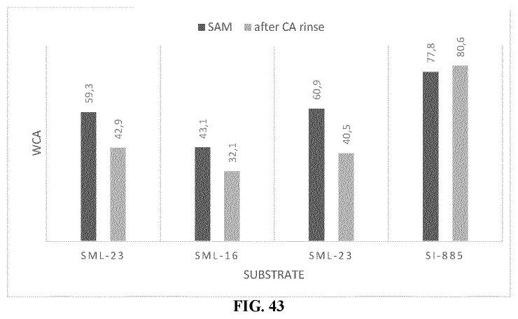

FIG. 43 selective SAM removal with CA rinse (SML-23. SML-16. SML-23)

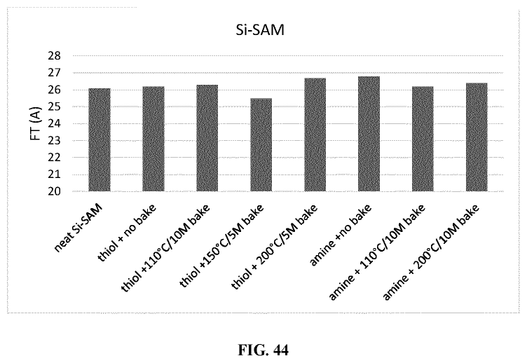

FIG. 44 Effect of chemical rinse and thermal treatment on Si-SAM (SML-12)

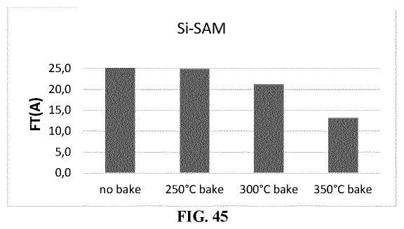

FIG. 45 Effect of temperature on SAM stability on Si (SML-12)

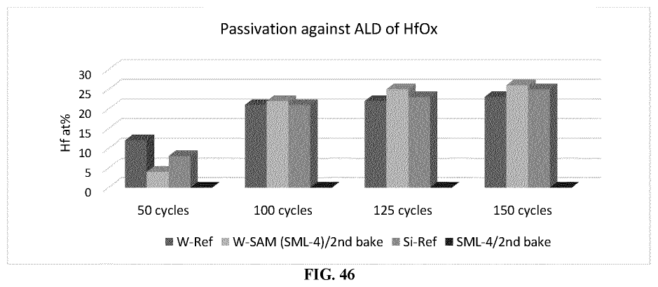

FIG. 46 SAM (SML-4) passivation properties against ALD of HfOX

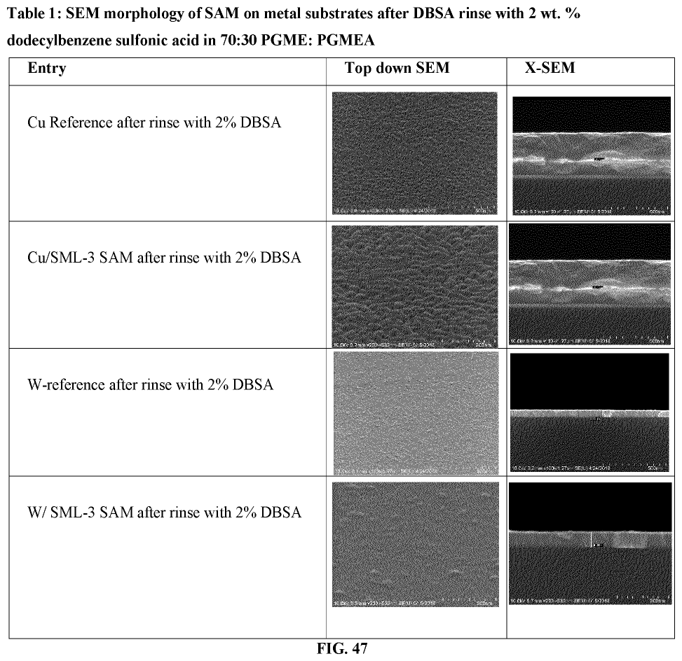

FIG. 47 Table 7: SEM morphology of SAM on metal substrates after DBSA rinse with 2 wt. % dodecylbenzene sulfonic acid in 70:30 PGME:PGMEA

### SUMMARY OF THE INVENTION

This invention describes synthesis and processing of spin-coat able SAM and selective SAM deposition on dielectric surfaces, and metal surface. Structural design with multi-tether functional groups allows spin-coat ability and process conditions allow formation of well-defined SAMs. The general description of the compounds with different anchoring/reactive groups with either aromatic or aliphatic core is shown in FIG. 1. By incorporation of single anchor group per chain, and more than two chains per molecule, spin-coat method delivers desired amount of SAM precursors on the substrate and baking at higher temperature for short period allows well-defined self-assembled monolayers with very good packing density. Obtained dielectric SAM shows very good passivation against atomic layer deposition of hafnium oxide. Modification of SAM tail groups, polar vs non-polar allows selective pinning or guiding surface for BCP wetting for directed self-assembly. Anchor groups such as diethyl-phosphonate, thiols, azide and amine allow single step metal selective SAM deposition. However, metal-selective functional groups such as thiols, phosphonic acid/ester, amines also show some cross-grafting on dielectric surface when grafting temperature >150° C., a key step for spin-coat assembly procedure. Functional groups typically used for dielectric grafting such as chlorosilanes, alkoxysilane, hydroxyl, alkene, and alkyne react non-discriminately on dielectric vs metal surface. Hence, a process of selective chemical cleavage on the metal regions would provide enhanced dielectric grafting or coverage to enable subsequent metal grafting or coverage.

This invention describes new SAM precursor compounds and a new method for introducing selective chemical cleavage of organic moieties on metal substrates via spin-coatable reagents that consists of organic acid with specific pH 1-4 range in organic medium containing alcohol and ether/ester solvents. Selective area chemical grafting is difficult for dielectric regions with any organic functional groups. However, there are specific organic functional groups that can be used to interact with metal surfaces and graft or cover the regions via chemical reactions either covalently or coordinately. The general organic compounds such as self-assembled monolayer (SAM) and polymer brushes terminated with different anchoring/reactive groups that are specific for metal surfaces such as diethyl-phosphonate, thiols, azide and amine allow single step metal selective grafting/deposition at optimum annealing condition. These metal selective functional groups are useful for direct grafting process as shown in FIG. 2.

One aspect of this invention is a compound of structure (I), wherein A is a core moiety which is selected from structure (Ia), (Ib), (Ic) and (Id), to which is attached through X, which is either a direct valence bond or a divalent linking group, m number of linear alkylene moieties of chain length n, wherein n ranges from 8 to 12 and where each said linear alkylene moiety has a terminal B reactive moiety, and further wherein * designates possible attachment point of said linear alkylene moieties in each structure, wherein structure (Ia), and (Ib), have 2 or 3 attachment points (m=2 or 3) which are on adjacent carbons, structure (Ic) has 2 attachment point (m=2), structure (Id), has three attachment point (m=3).

B is selected from —OH, —CH═CH₂, —O—(P=O)(OR)₂, —O—(P=O)(OR)Rs, N₃, and —SH, wherein R and Rs are independently selected from is a C-1 to C-8 alkyl.

R₁ₐ and R_{1b} are independently selected from H a C-1 to C-4 alkyl, a C-1 to C-4 alkyloxy, a C-3 to C-6, methyl(carbonyloxyalkyl) (—CH₂—(C=O)—O-alkyl), a C-2 to C-5 carbonyloxyalkyl (—(C=O)—O-alkyl), nitro (NO₂), NH₂, and CN.

R_{1c}, R_{1d}, R₁ₑ, R_{1f}, and R_{1g} are independently selected from H a C-1 to C-4 alkyl, a C-1 to C-4 alkyloxy, C-2 to C-5 carbonyloxyalkyl (—(C=O)—O-alkyl), a C-3 to C-6 methylcarbonyloxyalkyl (—CH₂—(C=O)—O-alkyl), and CN.

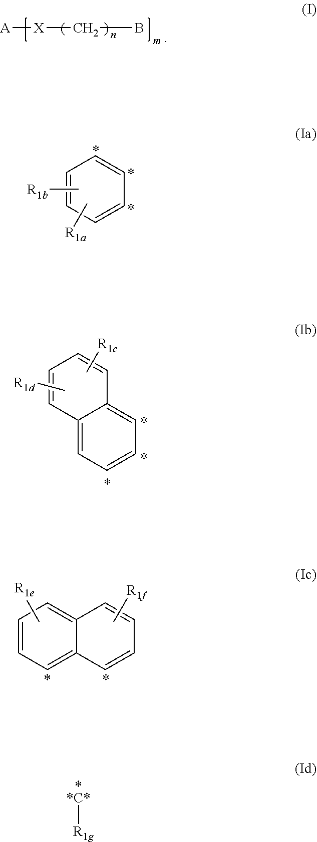

Another aspect of this invention pertains to composition comprising these compounds and the use these composition to form self-assembled monolayers (SAM) which may be used in DSA processing either as a neutral, polystyrene pinning or polymethylmethacrylate pinning layers in the directed self-assembly of overlying block copolymers and the process of selectively cleaving these (SAM) on metal substrates and the remover composition for affecting this removal.

### DETAILED DESCRIPTION OF THE INVENTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including,” as well as other forms such as “includes” and “included”, is not limiting. Also, terms such as “element” or “component” encompass both elements and components comprising one unit and elements or components that comprise more than one unit, unless specifically stated otherwise. As used herein, the conjunction “and” is intended to be inclusive and the conjunction “or” is not intended to be exclusive unless otherwise indicated. For example, the phrase “or, alternatively” is intended to be exclusive. As used herein, the term “and/or” refers to any combination of the foregoing elements including using a single element.

The term C-1 to C-4 alkyl embodies methyl and C-2 to C-4 linear alkyls and C-3 to C-4 branched alkyl moieties, for example as follows: methyl(-CH₃), ethyl (—CH₂—CH₃), n-propyl (—CH₂—CH₂—CH₃), isopropyl (—CH(CH₃)₂, n-butyl (—CH₂—CH₂—CH₂—CH₃), tert-butyl (—C(CH₃)₃), isobutyl (CH₂—CH(CH₃)₂, 2-butyl (—CH(CH₃)CH₂—CH₃). Similarly, the term C-1 to C-8 embodies methyl C-2 to C-8 linear, C-3 to C-8 branched alkyls, C-4 to C-8 cycloalkyls (e.g., cyclopentyl, cyclohexyl etc) or C-5-C-8 alkylenecycloalkyls (e.g., —CH₂-cyclohexyl, CH₂—CH₂-cyclopentyl etc.

The term C-2 to C-5 alkylene embodies C-2 to C-5 linear alkylene moieties (e.g., ethylene, propylene etc.) and C-3 to C-5 branched alkylene moieties (e.g., —CH(CH₃)—, —CH(CH₃)—CH₂—, etc.).

Di-block and triblock copolymers of styrenic and alkyl 2-methylenealkanoate derived repeat unit moieties useful as components in the inventive compositions described herein may be made by a variety of methods, such as anionic polymerization, atom transfer radical polymerization (ATRP), Reversible addition-fragmentation chain transfer (RAFT) polymerization, living radical polymerization and the like (Macromolecules 2019, 52, 2987-2994; Macromol. Rapid Commun. 2018, 39, 1800479; A. Deiter Shluter et al Synthesis of Polymers, 2014, Volume 1, p315; Encyclopedia of Polymer Science and Technology, 2014, Vol 7, p 625.)

The random copolymer poly(styrene-co-methyl methacrylate) is abbreviated as “P(S-co-MMA),” and the oligomeric version of this materials is abbreviated P(S-co-MMA). Similarly the block copolymer poly(styrene-block-methyl methacrylate) is abbreviated as P(S-b-MMA), while the oligomer of this material is abbreviated as oligo(S-b-MMA). The oligomer oligo(styrene-co-p-octylstyrene)-block-(methyl methacrylate-co- di(ethylene glycol) methyl ether methacrylate) uses the same abbreviations to designate random block copolymer elements, specifically oligo(S-co-p-OS)-b-P(MMA-co-DEGMEMA), in which S=styrene, p-OS=para-octylstyrene, MMA=methacrylate, DEGMEMA=di(ethylene glycol) methyl ether methacrylate designate the repeat units in this block copolymer whose two blocks are random copolymers.

FOV is the abbreviation for field of view for top-down scanning electron micrographs (SEM) for the SEM FIGs. in this application. “L/S,” is an abbreviation for line and space lithographic features.

PGMEA and PGME are respectively abbreviations for 1-methoxypropan-2-yl acetate and 1-methoxypropan-2-ol.

The abbreviation PMMA is an abbreviation for poly(methyl methacrylate).

The term PMMA affinity brush refers to a polymer which is polar, has a narrow polydispersity and have a grafting group at one chain end and can thus become attached spin casting from a solution this polymer to form a grafted polar brush which has an affinity to polar PMMA polymer block segments, in a block copolymer such as a block copolymer of styrene and methyl methacrylate. Examples of such materials are poly(methyl methacrylate) polymers or other polar alkyl methacrylate which have a reactive end group at one end such as an alcohol end group (e.g. alkyl alcohol or benzyl alcohol end group), and have a narrow polydispersity (e.g. 1 to to about 1.1), and whose reactive end group can interact with IC (integrated circuit) substrates such as Silicon dioxide grafting this polymer with the reactive end group to this substrate forming for instance a surface which has a brush of PMMA polymers attached to it.

It is to be understood that both the foregoing general description and the following detailed description are illustrative and explanatory and are not restrictive of the subject matter as claimed. In this application, the use of the singular includes the plural, the word “a” or “an” means “at least one,” and the use of “or” means “and/or,” unless specifically stated otherwise. Furthermore, the use of the term “including,” as well as other forms such as “includes” and “included,” is not limiting. Also, terms such as “element” or “component” encompass both elements and components comprising one unit and elements or components that comprise more than one unit, unless specifically stated otherwise. As used herein, the conjunction “and” is intended to be inclusive and the conjunction “or” is not intended to be exclusive unless otherwise indicated. For example, the phrase “or, alternatively” is intended to be exclusive. As used herein, the term “and/or” refers to any combination of the foregoing elements including using a single element.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature references and similar materials defines a term in a manner that contradicts the definition of that term in this application, this application controls.

Unless otherwise indicated, “alkyl” refers to hydrocarbon groups which can be linear, branched (e.g., methyl, ethyl, propyl, isopropyl, tert-butyl and the like) or cyclic (e.g., cyclohexyl, cyclopropyl, cyclopentyl and the like) multicyclic (e.g., norbornyl, adamantyl and the like). These alkyl moieties may be substituted or unsubstituted as described below. The term “alkyl” refers to such moieties with C-1 to C-8 carbons. It is understood that for structural reasons linear alkyls start with C-1, while branched alkyls and cyclic alkyls start with C-3 and multicyclic alkyls start with C-5. Moreover, it is further understood that moieties derived from alkyls described below, such as alkyloxy and perfluoroalkyl, have the same carbon number ranges unless otherwise indicated. If the length of the alkyl group is specified as other than described above, the above-described definition of alkyl still stands with respect to it encompassing all types of alkyl moieties as described above and that the structural consideration with regards to minimum number of carbons for a given type of alkyl group still apply.

Alkyloxy (a.k.a. Alkoxy) refers to an alkyl group on which is attached through an oxy (—O—) moiety (e.g., methoxy, ethoxy, propoxy, butoxy, 1,2-isopropoxy, cyclopentyloxy cyclohexyloxy and the like). These alkyloxy moieties may be substituted or unsubstituted as described below.

Halo or halide refers to a halogen, F, Cl, Br or I which is linked by one bond to an organic moiety.

As used herein the term lactone encompasses both mono-lactones (e.g., caprolactone) and di-lactones (e.g., lactide).

Haloalkyl refers to a linear, cyclic or branched saturated alkyl group such as defined above in which at least one of the hydrogens has been replaced by a halide selected from the group of F, Cl, Br, I or mixture of these if more than one halo moiety is present. Fluoroalkyls are a specific subgroup of these moieties.

Perfluoroalkyl refers to a linear, cyclic or branched saturated alkyl group as defined above in which the hydrogens have all been replaced by fluorine (e.g., trifluoromethyl, pefluoroethyl, prefluoroisopropyl, perfluorocyclohexyl and the like).

One aspect of this invention is a compound of structure (I), wherein A is a core moiety which is selected from structure (Ia), (Ib), (Ic) and (Id), to which is attached through X, which is either a direct valence bond or a divalent linking group, m number of linear alkylene moiety of chain length n, wherein n ranges from 8 to 12 and where each said linear alkylene moiety has a terminal B reactive moiety, and further wherein * designates possible attachment point of said linear alkylene moieties in each structure, wherein structure (Ia), and (Ib), have 2 or 3 attachment points (m=2 or 3) which are on adjacent carbons, structure (Ic) has 2 attachment point (m=2), structure (Id), has three attachment point (m=3).

B is selected from —OH, —CH═CH₂, —O—(P=O)(OR)₂, —O—(P=O)(OR)Rs, N₃, and —SH, wherein R and Rs are independently selected from is a C-1 to C-8 alkyl.

R₁ₐ and R_{1b} are independently selected from H a C-1 to C-4 alkyl, a C-1 to C-4 alkyloxy, a C-3 to C-6, methyl(carbonyloxyalkyl) (—CH₂—(C=O)—O-alkyl), a C-2 to C-5 carbonyloxyalkyl (—(C=O)—O-alkyl), nitro (NO₂), NH₂, and CN.

R_{1c}, R_{1d}, R₁ₑ,R_{1f}, and R_{1g} are independently selected from H a C-1 to C-4 alkyl, a C-1 to C-4 alkyloxy, C-2 to C-5 carbonyloxyalkyl (—(C=O)—O-alkyl), a C-3 to C-6 methylcarbonyloxyalkyl (—CH₂—(C=O)—O-alkyl), and CN.

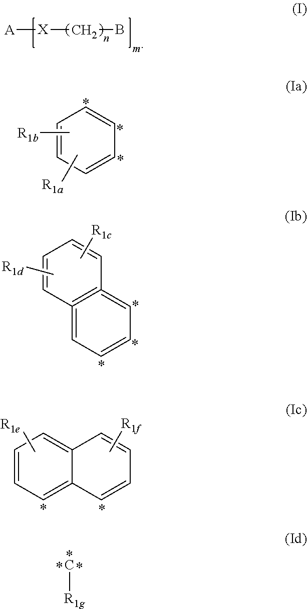

In another aspect of the inventive compound of structure (I), X is a direct valence bond.

In another aspect of the inventive compound of structure (I), X is a divalent linking group.

In one aspect of the inventive compound of structure (I), of when X is a divalent linking group and when also the core moieties are (Ia), (Ib), or (Ic), the divalent linking group for is selected is selected from the group consisting of oxy (—O—), oxycarbonyl (—O—C(═O)—), carbonyloxy(—C(═O)—O—), carbonyl (—(C=O)—), sulfinyl (—(S(═O))—), and sulfone (—S(═O)₂—).

In one aspect of the inventive compound of structure (I), of when X is a divalent linking group and when also the core moiety is (Id), the divalent linking group is selected from the group consisting of oxy (—O—), oxycarbonyl (—O—C(═O)—), carbonyloxy(—C(═O)—O—), ethyne

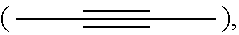

1,4-phenylene(-Ph-), 1,4-phenyleneoxy(-Ph-O—), 1,4-oxyphenylene(—O-Ph-), carbonyl (—(C=O)—), sulfinyl (—(S(=O))—), sulfone (—S(═O)₂—), 1,2-ethene(-CH═CH—), 1,1-ethene (—C(═CH₂)—), and methylene (—CH₂—).

In another aspect of the inventive compound of structure (I), of when X is a divalent linking group and when also the core moieties are (Ia), (Ib), (Ic) or (Id), said divalent linking group is oxy (—O—). In another aspect of this embodiment said divalent linking group is oxycarbonyl (—O—C(═O)—). In another aspect of this embodiment said divalent linking group is carbonyloxy(—C(═O)—O—). In another aspect of this embodiment said divalent linking group is carbonyl (—(C=O)—). In another aspect of this embodiment said divalent linking group is sulfinyl (—(S(═O))—). In another aspect of this embodiment said divalent linking group is sulfone (—S(=O)₂—).

In another aspect of the inventive compound of structure (I), of when X is a divalent linking group and when also the core moiety is (Id) said divalent linking group is ethyne

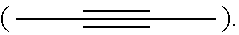

In another aspect of this embodiment said divalent linking group is 1,4-phenylene(-Ph-). In another aspect of this embodiment said divalent linking group is 1,4-phenyleneoxy(-Ph-O—). In another aspect of this embodiment said divalent linking group is 1,4-oxyphenylene(—O-Ph-). In another aspect of this embodiment said divalent linking group is 1,2-ethene(-CH═CH—). In another aspect of this embodiment said divalent linking group is 1,1-ethene (—C(═CH₂)—). In another aspect of this embodiment said divalent linking group is methylene.

In a specific aspect of the inventive compound described above A has structure (Ia). In one aspect of this embodiment, structure (Ia) has m=2 and it has the more specific structure (Ia-1) where * designates the position of the two attachment points; in a more specific aspect of this embodiment, it has structure (Ia-2).

In another aspect of this embodiment structure compound (I) has m=3 and has structure (Ia), which has three attachment points at the positions designated by * in structure (Ia); in a more specific aspect of this embodiment, it has structure (Ia-3), where * designates the positions of the attachment points. In one aspect of this embodiment, R₁ₐ and R_{1b} are H. In another embodiment at least one of R₁ₐ and R_{1b} is H. In another aspect at least one of R₁ₐ and R_{1b} is a C-1 to C-4 alkyl. In another aspect of this embodiment at least one of R₁ₐ and R_{1b} is C-1 to C-4 alkyloxy. In another aspect of this embodiment at least one of R₁ₐ and R_{1b} is a C-2 to C-5 carbonyloxyalkyl (—(C=O)—O-alkyl). In yet another embodiment at least one of R₁ₐ and R_{1b} is a C-3 to C-6, methyl(carbonyloxyalkyl) (—CH₂—(C=O)—O-alkyl). In still another aspect of this embodiment at least one of R₁ₐ and R_{1b} is NO₂. In still another aspect of this embodiment, at least one of R₁ₐ and R_{1b} is NH₂. In yet another aspect of this embodiment at least one of R₁ₐ and R_{1b} is CN. In another aspect of this embodiment both R₁ₐ and R_{1b} are the same substituent as previously designated. In another aspect of this embodiment both R₁ₐ and R_{1b} are different substituents as previously designated. In another aspect of this embodiment both R₁ₐ and R_{1b} are not H and are the same substituents. In another aspect of this embodiment both R₁ₐ and R_{1b} are not H and are different substituents.

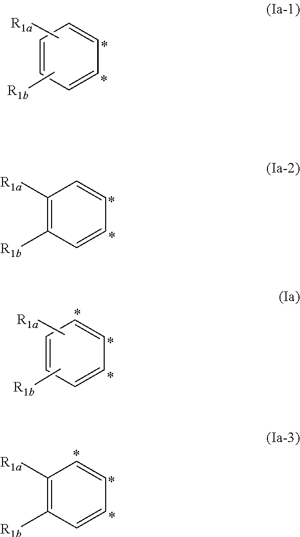

In a specific aspect of the inventive compound of structure (I), described herein, A has structure (Ib). In one aspect of this embodiment the compound of structure (I) m=2, structure (Ib) has the more specific structure (Ib-1), where * designates the position of the 2 attachment points; in another aspect of this embodiment, it has the more specific structure (Ib-2), where * designates the position of the 2 attachment points. In one aspect of this embodiment at least one of R_{1c} and R₁ₐ are H. In another aspect at least one of R_{1c} and R₁ₐ is a C-1 to C-4 alkyl. In another aspect of this embodiment at least one of R_{1c} and R₁ₐ is C-1 to C-4 alkyloxy. In another aspect of this embodiment at least one of R_{1c} and R₁ₐ is a C-2 to C-5 carbonyloxyalkyl (—(C=O)—O-alkyl). In yet another embodiment at least one of R_{1c} and R₁ₐ is a C-3 to C-6, methyl(carbonyloxyalkyl) (—CH₂—(C=O)—O-alkyl). In still another aspect of this embodiment at least one of R_{1c} and R₁ₐ is NO₂. In still another aspect of this embodiment, at least one of R_{1c} and R₁ₐ is NH₂. In yet another aspect of this embodiment at least one of R_{1c} and R₁ₐ is CN. In another aspect of this embodiment both R_{1c} and R₁ₐ are the same substituent as previously designated. In another aspect of this embodiment, R_{1c} and R₁ₐ are different substituents as previously designated. In one specific embodiment both R_{1c} and R₁ₐ are H.

In another aspect of the inventive compound of structure (I), m=2 with A having structure (Ib), it has the more specific structure (Ib-1), where * designates the position of the 2 attachment points; in another aspect of this embodiment, it has the more specific structure (Ib-2), where * designates the position of the 2 attachment points. In one aspect of this embodiment at least one of R_{1c} and R₁ₐ are H. In another aspect at least one of R_{1c} and R₁ₐ is a C-1 to C-4 alkyl. In another aspect of this embodiment at least one of R_{1c} and R_{1d} is C-1 to C-4 alkyloxy. In another aspect of this embodiment at least one of R_{1c} and R_{1d} is a C-2 to C-5 carbonyloxyalkyl (—(C=O)—O-alkyl). In yet another embodiment at least one of R_{1c} and R_{1d} is a C-3 to C-6, methyl(carbonyloxyalkyl) (—CH₂—(C=O)—O-alkyl). In still another aspect of this embodiment at least one of R_{1c} and R_{1d} is NO₂. In still another aspect of this embodiment, at least one of R_{1c} and R_{1d} is NH₂. In yet another aspect of this embodiment at least one of R_{1c} and R₁ₐ is CN. In another aspect of this embodiment both R_{1c} and R₁ₐ are the same substituent as previously designated. In another aspect of this embodiment, R_{1c} and R₁ₐ are different substituents as previously designated. In one specific embodiment both R_{1c} and R_{1d} are H.

In another aspect of the inventive compound of structure (I), m=3 with A having structure (Ib), where * designates the position of the 3 attachment points; in another aspect of this embodiment, it has the more specific structure (Ib-3), where * designates the position of the 3 attachment points. In one aspect of this embodiment at least one of R_{1c} and R₁ₐ are H. In another aspect at least one of R_{1c} and R_{1d} is a C-1 to C-4 alkyl. In another aspect of this embodiment at least one of R_{1c} and R_{1d} is C-1 to C-4 alkyloxy. In another aspect of this embodiment at least one of R_{1c} and R_{1d} is a C-2 to C-5 carbonyloxyalkyl (—(C=O)—O-alkyl). In yet another embodiment at least one of R_{1c} and R_{1d} is a C-3 to C-6, methyl(carbonyloxyalkyl) (—CH₂—(C=O)—O-alkyl). In still another aspect of this embodiment at least one of R_{1c} and R_{1d} is NO₂. In still another aspect of this embodiment, at least one of R_{1c} and R_{1d} is NH₂. In yet another aspect of this embodiment at least one of R_{1c} and R_{1d} is CN. In another aspect of this embodiment both R_{1c} and R_{1d} are the same substituent as previously designated. In another aspect of this embodiment, R_{1c} and R_{1d} are different substituents as previously designated. In one specific embodiment both R_{1c} and R₁ₐ are H.

In another aspect of the inventive compound of structure (I), m=3 with A having structure (Ib), where * designates the position of the 3 attachment points; in another aspect of this embodiment, it has the more specific structure (Ib-4), where * designates the position of the 3 attachment points. In one aspect of this embodiment at least one of R_{1c} and R₁ₐ are H. In another aspect at least one of R_{1c} and R_{1d} is a C-1 to C-4 alkyl. In another aspect of this embodiment at least one of R_{1c} and R_{1d} is C-1 to C-4 alkyloxy. In another aspect of this embodiment at least one of R_{1c} and R_{1d} is a C-2 to C-5 carbonyloxyalkyl (—(C=O)—O-alkyl). In yet another embodiment at least one of R_{1c} and R_{1d} is a C-3 to C-6, methyl(carbonyloxyalkyl) (—CH₂—(C=O)—O-alkyl). In still another aspect of this embodiment at least one of R_{1c} and R₁ₐ is NO₂. In still another aspect of this embodiment, at least one of R_{1c} and R_{1d} is NH₂. In yet another aspect of this embodiment at least one of R_{1c} and R_{1d} is CN. In another aspect of this embodiment both R_{1c} and R_{1d} are the same substituent as previously designated. In another aspect of this embodiment, R_{1c} and R_{1d} are different substituents as previously designated. In one specific embodiment both R_{1c} and R₁ₐ are H.

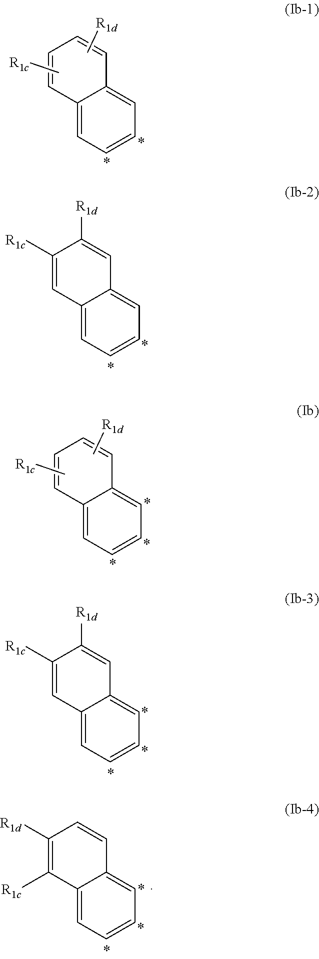

In a specific aspect of the inventive compound of structure (I) A has structure (Ic). In one aspect of this embodiment at least one of R₁ₑ and R_{1f} is H. In another aspect of this embodiment at least one of R₁ₑ and R_{1f} is a C-1 to C-4 alkyl. In one aspect of this embodiment A has the more specific structure (Ic-1). In another aspect of this embodiment, it has the more specific structure (Ic-2). In still another aspect of this embodiment, at least one of R₁ₑ and R_{1f} is C-1 to C-4 alkyloxy. In still another aspect of these embodiment at least one of R₁ₑ and R_{1f} is a C-2 to C-5 carbonyloxyalkyl (—(C=O)—O-alkyl). In still another aspect of these embodiment at least one of R₁ₑ and R_{1f} is a C-3 to C-6, methyl(carbonyloxyalkyl) (—CH₂—(C=O)—O-alkyl). In yet another aspect at least one of R₁ₑ and R_{1f} is NO₂. In still another aspect at least one of R₁ₑ and R_{1f} is NH₂. In still another at least one of Rₑ and R_{1f} is CN. In another aspect of this embodiment both R₁ₑ and R_{1f} are the same substituent as previously designated. In another aspect of this embodiment, they are different substituents as previously designated. In one specific embodiment both R₁ₑ and R_{1f} are H.

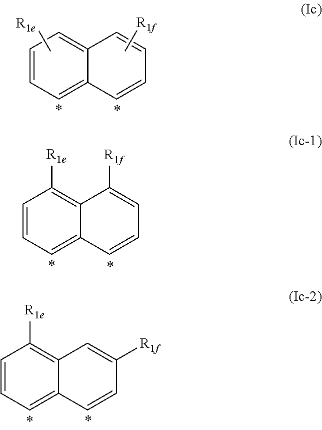

In a specific aspect of the inventive compound (I), described herein, A has structure (Id). In one aspect of this embodiment, X is a methylene linking group and the combination of A and X has structure (Id-1), wherein * designates the attachment point of the alkylene moieties. In another aspect of this embodiment, X is a 1,4-phenylene linking group, and the combination of A and X has structure (Id-2, wherein * designates the attachment point of said linear alkylene moieties. In yet another aspect of this embodiment, X is a 1,4-phenyleneoxy (-Ph-O—) linking group, and the combination of A and X has structure (Id-3), wherein * designates the attachment point of said linear alkylene moieties. In yet another aspect of these embodiments R_{1g} is a C-1 to C-4 alkyloxy. In still another embodiment of this aspect R_{1g} is a C-2 to C-5 carbonyloxyalkyl (—(C=O)—O-alkyl). In yet another aspect of this embodiment R_{1g} is a C-3 to C-6, methyl(carbonyloxyalkyl) (—CH₂—(C=O)—O-alkyl). In still another aspect of this embodiment R_{1g} is NO₂. In yet another embodiment of this aspect R_{1g} is NH₂. In still another aspect of this embodiment R_{1g} is CN. In still another aspect of this embodiment R_{1g} is H.

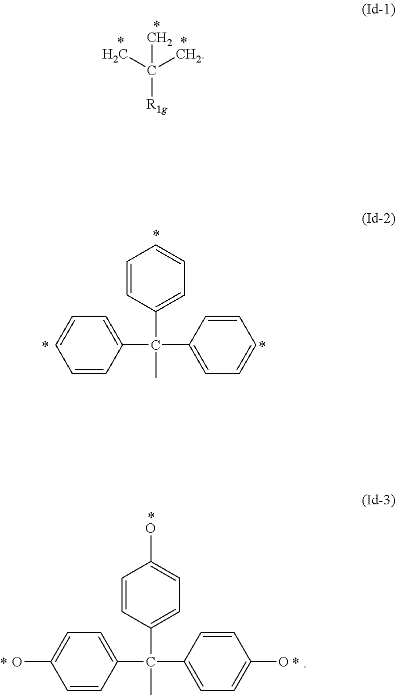

In one aspect of the embodiment of the inventive compound of structure (I) described herein, n ranges from 9 to 12.

In one aspect of the embodiment of the inventive compound of structure (I) described herein B is —OH.

In one aspect of the embodiment of the inventive compound of structure (I) described herein B is —CH═CH₂

In one aspect of the embodiment of the inventive compound of structure (I) described herein B is —O—(P=O)(OR)₂.

In one aspect of the embodiment of the inventive compound of structure (I) described herein B is —O—(P═O)(OR)Rₛ.

In one aspect of the embodiment of the inventive compound of structure (I) described herein wherein O—(P=O)(OR)₂, is present either as at least one substituent R₁ₐ or R_{1b} in structure (Ia), at least one of substituents R_{1d} or R_{1c} in structure (Ib), at least one of substituent R_{1f} or R₁ₑ in structure (Ic) or as substituent R_{g} in structure (Id), R is ethyl or methyl. In one aspect of this embodiment R is ethyl. In another aspect of this embodiment R is methyl.

In one aspect of the embodiment of the inventive compound of structure (I) described herein wherein —O—(P=O)(OR)Rs, is present either as at least one substituent R₁ₐ or R_{1b} in structure (Ia), at least one of substituents R_{1d} or R_{1c} in structure (Ib), at least one of substituent R_{1f} or R₁ₑ in structure (Ic) or as R_{g} in structure (Id) R is ethyl or methyl. In one aspect of this embodiment R is ethyl. In another aspect of this embodiment R is methyl. In another aspect of this embodiment Rs is ethyl. In yet another aspect of this embodiment Rs is methyl. In one aspect of this embodiment both R and Rs are methyl. In another aspect of this embodiment both R and Rs are ethyl.

In another aspect of the inventive compound of structure (I), described herein B is —N₃.

In another aspect of the inventive compound of structure (I), described herein B is —SH.

In another aspect of this invention is a compound selected from the following the compounds: 11,11′,11″-((ethane-1,1,1-triyltris(benzene-4,1-diyl))tris(oxy))tris(undecan-1-ol) (M-1), 11,11′,11″-(propane-1,2,3-triyltris(oxy))tris(undecan-1-ol) (M-2), (11,11′,11″-(benzene-1,2,3-triyltris(oxy))tris(undecan-1-ol).(M-3), (11,11′-(1,2-phenylenebis(oxy))bis(undecan-1-ol). (M-4), 11,11′-(naphthalene-2,3-diylbis(oxy))bis(undecan-1-ol(M-5), 11,11′-(naphthalene-1,8-diylbis(oxy))bis(undecan-1-ol (M-6), 11,11′-(1,2-phenylenebis(oxy))bis(undecan-1-ol (M-7), 11,11′-((4-(tert-butyl)-1,2-phenylene)bis(oxy))bis(undecan-1-ol) (M-8), 1,2-phenylenebis(oxy))bis(hexadecan-1-ol (M-9), 3,4-bis((11-hydroxyundecyl)oxy)benzonitrile (M-10), 11-(2,6-bis(undec-10-en-1-yloxy)phenoxy)undecan-1-ol (M-11), 10,10′-(1,2-phenylenebis(oxy))bis(decan-1-ol (M-12), 11,11′-(1,2-phenylenebis(oxy))bis(undecane-1-thiol) (M-13), 1,2-bis((11-azidoundecyl)oxy)benzene (M-14), tetraethyl ((1,2-phenylenebis(oxy))bis(undecane-11,1-diyl))bis(phosphonate) (M-15), 11,11′-((4-methyl-1,2-phenylene)bis(oxy))bis(undecan-1-ol) (M-16), methyl 3,4-bis((11-hydroxyundecyl)oxy)benzoate (M-17), 11,11′-((4-nitro-1,2-phenylene)bis(oxy))bis(undecan-1-ol) (M-18), dimethyl 4,5-bis((11-hydroxyundecyl)oxy)phthalate (M-19), 3,4-bis((11-hydroxyundecyl)oxy)benzonitrile (M-20), (E)-11,11′-((4-(hex-1-en-1-yl)-1,2-phenylene)bis(oxy))bis(undecan-1-ol) (M-21), tetraethyl (((4-cyano-1,2-phenylene)bis(oxy))bis(undecane-11,1-diyl))bis(phosphonate) (M-22), (tetraethyl (((4-acetyl-1,2-phenylene)bis(oxy))bis(undecane-11,1-diyl))bis(phosphonate (M-23), dimethyl 4,5-bis((11-(diethoxyphosphoryl)undecyl)oxy)phthalate (M-24), and (tetraethyl (((4-nitro-1,2-phenylene)bis(oxy))bis(undecane-11,1-diyl))bis(phosphonate)) (M-25).

### Inventive SAM Composition

Another aspect of this invention is a SAM precursor composition comprising any one inventive compound as described herein and an organic spin casting solvent which can be a single solvent or a mixture of at least two solvents.

In one aspect of this inventive composition, it consists essentially of any one of these compounds and said organic spin casting solvent. In another aspect of this embodiment, it consists of any one of these compounds and said organic spin casting solvent. In one aspect of this embodiment said composition consists of said inventive compounds where the organic spin casting solvent is a single solvent. In another aspect said organic spin casting solvent is a mixture of at least two solvents.

### Inventive PMMA Affinity SAM Composition

Another aspect of this invention is an inventive composition which is a SAM precursor composition which forms a polar SAM on coating on a substrate which has an affinity toward polar block segment in a block copolymer (e.g., poly(methyl methacrylate (PMMA) block segment) which is comprised of an organic spin casting solvent and one Inventive PMMA SAM precursor compounds as follows:
An inventive compound selected from: An inventive compound containing structure (Ia), wherein at least one of R₁ₐ and R_{1b} is a polar substituent selected from C-1 to C-4 alkyloxy, a C-3 to C-6, methyl(carbonyloxyalkyl) (—CH₂—(C=O)—O-alkyl), a C-2 to C-5 carbonyloxyalkyl (—(C=O)—O-alkyl), a Nitro (NO₂), NH₂, and CN, were either R₁ₐ, or R_{1b} which is not one of these substituent is either H or a C-1 to C-4 alkyl, preferably H.
An inventive compound containing structure (Ib), wherein at least one of R_{1c} or R₁ₐ is a polar substituent independently selected from a C-1 to C-4 alkyloxy, a C-3 to C-6, methyl(carbonyloxyalkyl) (—CH₂—(C=O)—O-alkyl), a C-2 to C-5 carbonyloxyalkyl (—(C=O)—O-alkyl), and CN, and further were either R_{1c}, or R₁ₐ which is not one of these substituent is either H or a C-1 to C-4 alkyl, preferably H.
An inventive compound containing structure (Ic), wherein at least one of R₁ₑ, or R_{1f}, is a polar substituent independently selected from a C-1 to C-4 alkyloxy, a C-3 to C-6, methyl(carbonyloxyalkyl) (—CH₂—(C=O)—O-alkyl), a C-2 to C-5 carbonyloxyalkyl (—(C=O)—O-alkyl), and CN and further were either R₁ₑ, or R_{1f} which is not one of these substituent is either H or a C-1 to C-4 alkyl, preferably H.
An inventive compound containing structure (Id), wherein R_{1g} is a polar substituent selected from a C-1 to C-4 alkyloxy, a C-3 to C-6, methyl(carbonyloxyalkyl) (—CH₂—(C=O)—O-alkyl), a C-2 to C-5 carbonyloxyalkyl (—(C=O)—O-alkyl), and CN.

### Inventive PS Affinity SAM Composition

Another aspect of this invention is a composition which is a SAM precursor composition which forms a non-polar SAM on coating on a substrate which has an affinity toward non-polar block segment in a block copolymer (e.g., polystyrene (PS) block segment) which is comprised of an organic spin casting solvent and one Inventive PS SAM precursor compounds as follows:
An inventive compound containing structure (Ia), wherein at least one of R₁ₐ and R_{1b} is a non-polar substituent selected from a C-1 to C-4 alkyl, and further were either R₁ₐ, or R_{1b} which is not one of these non-polar substituents is H.
An inventive compound containing structure (Ib), wherein at least one of R_{1c} or R_{1d} is a non-polar substituent independently selected from a C-1 to C-4 alkyl and further were either R_{1c} or R₁ₐ which is not one of these non-polar substituent is H.
An inventive compound containing structure (Ic), wherein at least one of R₁ₑ or R_{1f}, is a non-polar substituent independently selected from a C-1 to C-4 alkyl and further were either R₁ₑ, or R_{1f} which is not one of these non-polar substituents is H.
An inventive compound containing structure (Id), wherein R_{1g} is a non-polar substituent selected from a C-1 to C-4 alkyl.

### Inventive Neutral Affinity SAM Composition

Another aspect of this invention is a composition which is a SAM precursor composition which forms a neutral SAM on coating on a substrate which has a neutral affinity toward non-polar block segment and polar block segment a block copolymer (e.g., polystyrene (PS) block segment and poly(methyl methacrylate block (PMMA) block segment is a PS-b-PMMA polymer) which is comprised of an organic which is comprised of an organic spin casting solvent and one Inventive Neutral affinity SAM precursor compound as follows:
An inventive compound containing structure (Ia), wherein R₁ₐ and R_{1b} are both H.
An inventive compound containing structure (Ib), wherein both R_{1c} and R_{1d} are H.
An inventive compound containing structure (Ic), wherein both R₁ₑ and R_{1f} are H.
An inventive compound containing structure (Id), wherein R_{1g} is H.

### Spin Casting Solvent for Inventive SAM Compositions

Suitable solvents for use as an organic spin casting solvent are any solvent which is employed to spin cast materials such as photoresist, bottom antireflective coatings or other types of organic coatings using the lithographic processing of semiconductor materials which can dissolve the Inventive compounds. Non limiting examples. In another aspect of said novel composition, the organic spin casting solvent is one which can dissolve the inventive compounds. This organic spin casting solvent may be a single solvent or a mixture of solvents. Suitable solvents are organic solvent which may include, for example, a glycol ether derivative such as ethyl cellosolve, methyl cellosolve, propylene glycol monomethyl ether (PGME), diethylene glycol monomethyl ether, diethylene glycol monoethyl ether, dipropylene glycol dimethyl ether, propylene glycol n-propyl ether, or diethylene glycol dimethyl ether; a glycol ether ester derivative such as ethyl cellosolve acetate, methyl cellosolve acetate, or propylene glycol monomethyl ether acetate (PGMEA); carboxylates such as ethyl acetate, n-butyl acetate and amyl acetate; carboxylates of di-basic acids such as diethyloxylate and diethylmalonate; dicarboxylates of glycols such as ethylene glycol diacetate and propylene glycol diacetate; and hydroxy carboxylates such as methyl lactate, ethyl lactate (EL), ethyl glycolate, and ethyl-3-hydroxy propionate; a ketone ester such as methyl pyruvate or ethyl pyruvate; an alkyloxycarboxylic acid ester such as methyl 3-methoxypropionate, ethyl 3-ethoxypropionate, ethyl 2-hydroxy-2-methylpropionate, or methylethoxypropionate; a ketone derivative such as methyl ethyl ketone, acetyl acetone, cyclopentanone, cyclohexanone or 2-heptanone; a ketone ether derivative such as diacetone alcohol methyl ether; a ketone alcohol derivative such as acetol or diacetone alcohol; a ketal or acetal like 1,3 dioxalane and diethoxypropane; lactones such as butyrolactone; an amide derivative such as dimethylacetamide or dimethylformamide, anisole, and mixtures thereof. In one aspect of this embodiment said solvent may be PGMEA and mixture of PGMEA; and PGME. In one aspect of this embodiment said mixture of PGME; and PGMEA is a 70:30 wt:wt mixture.

In another aspect of these composition, said inventive compound comprise from about 0.5 wt. % to about 3.00 wt. % of the total weight of said composition including the organic spin casting solvent. In another aspect it comprises from about 0.75 wt. % to about 2.75 wt. %. In yet another embodiment it comprises from about 1.00 wt. % to about 2.5 wt. %. In yet another embodiment it comprises from about 1.5 wt. % to about 2.25 wt. %.

In another aspect of this inventive composition the compound is selected from the group consisting a compound selected from the following the compounds: 11,11′,11″-((ethane-1,1,1-triyltris(benzene-4,1-diyl))tris(oxy))tris(undecan-1-ol) (M-1), 11,11′,11″-(propane-1,2,3-triyltris(oxy))tris(undecan-1-ol) (M-2), (11,11′,11″-(benzene-1,2,3-triyltris(oxy))tris(undecan-1-ol).(M-3), (11,11′-(1,2-phenylenebis(oxy))bis(undecan-1-ol). (M-4), 11,11′-(naphthalene-2,3-diylbis(oxy))bis(undecan-1-ol(M-5), 11,11′-(naphthalene-1,8-diylbis(oxy))bis(undecan-1-ol (M-6), 11,11′-(1,2-phenylenebis(oxy))bis(undecan-1-ol (M-7), 11,11′-((4-(tert-butyl)-1,2-phenylene)bis(oxy))bis(undecan-1-ol) (M-8), 1,2-phenylenebis(oxy))bis(hexadecan-1-ol (M-9), 3,4-bis((11-hydroxyundecyl)oxy)benzonitrile (M-10), 11-(2,6-bis(undec-10-en-1-yloxy)phenoxy)undecan-1-ol (M-11), 10,10′-(1,2-phenylenebis(oxy))bis(decan-1-ol (M-12), 11,11′-(1,2-phenylenebis(oxy))bis(undecane-1-thiol) (M-13), 1,2-bis((11-azidoundecyl)oxy)benzene (M-14), tetraethyl ((1,2-phenylenebis(oxy))bis(undecane-11,1-diyl))bis(phosphonate) (M-15), 11,11′-((4-methyl-1,2-phenylene)bis(oxy))bis(undecan-1-ol) (M-16), methyl 3,4-bis((11-hydroxyundecyl)oxy)benzoate (M-17), 11,11′-((4-nitro-1,2-phenylene)bis(oxy))bis(undecan-1-ol) (M-18), dimethyl 4,5-bis((11-hydroxyundecyl)oxy)phthalate (M-19), 3,4-bis((11-hydroxyundecyl)oxy)benzonitrile (M-20), (E)-11,11′-((4-(hex-1-en-1-yl)-1,2-phenylene)bis(oxy))bis(undecan-1-ol) (M-21), tetraethyl (((4-cyano-1,2-phenylene)bis(oxy))bis(undecane-11,1-diyl))bis(phosphonate) (M-22), (tetraethyl (((4-acetyl-1,2-phenylene)bis(oxy))bis(undecane-11,1-diyl))bis(phosphonate (M-23), dimethyl 4,5-bis((11-(diethoxyphosphoryl)undecyl)oxy)phthalate (M-24), and (tetraethyl (((4-nitro-1,2-phenylene)bis(oxy))bis(undecane-11,1-diyl))bis(phosphonate)) (M-25).

In all the ranges described herein for the different components of this composition, these components are chosen such that their total equals 100 wt. %.

### Process of Coating an Inventive PMMA Affinity SAM Composition and Forming a PMMA Affinity SAM on a Substrate

Another aspect of this invention is of is a process of coating with any one of the inventive PMMA affinity SAM composition described herein and forming a PMMA affinity SAM, comprising the steps: i) coating a substrate with the polar affinity (a.k.a. PMMA affinity) SAM composition as described above,
ii) baking at a temperature ranging from about 150° C. to about 200° C.,
iii) rinsing with an organic spin casting solvent to form PMMA affinity SAM.
   The PMMA affinity SAM has an affinity to the PMMA block segments a block copolymer of polystyrene (PS) and methyl methacrylate (MMA) (PS-b-PMMA).

### Inventive PMMA SAM

Another aspect of this invention is the PMMA affinity SAM formed on a substrate by the process comprising steps i) to iii).

### Process of Coating an Inventive PS Affinity SAM Composition and Forming a PS Affinity SAM on a Substrate

Another aspect of this invention is of is a process of coating with a non-polar affinity SAM (a.k.a. PS block segment affinity) composition comprising the steps:
ia) coating a substrate with an inventive PS affinity SAM composition as described herein,
iia) baking at a temperature ranging from about 150° C. to about 200° C.,
iiia) rinsing with an organic spin casting solvent to form a self-assembled monolayer with an affinity to PS block segment of a block copolymer of polystyrene (PS) and methyl methacrylate (MMA)(PS-b-PMMA).

### Inventive PS SAM

Another aspect of this invention is the PS affinity SAM formed on a substrate by the process comprising steps ia) to iiia).

### Process of Coating an Inventive Neutral Affinity SAM Composition and Forming a Neutral Affinity SAM on a Substrate

Another aspect of this invention is of is a process of coating with a neutral affinity SAM (a.k.a. no affinity towards either PMMA or PS block segment in a PS-b-PMMA block copolymer) composition comprising the steps:
ib) coating a substrate with an inventive neutral affinity SAM composition as described herein,
iib) baking at a temperature ranging from about 150° C. to about 200° C.,
iiib) rinsing with an organic spin casting solvent to form a self-assembled monolayer with a neutral affinity block segment of a block copolymer of polystyrene (PS) and methyl methacrylate (MMA) (PS-b-PMMA).

### Inventive PS SAM

Another aspect of this invention is the Neutral affinity SAM formed on a substrate by the process comprising steps ib) to iiib).

### Process of Selective Deposition of an Inventive PMMA Affinity and an Inventive PS Affinity Self-Assembled Monolayer as Used in a Chemoepitaxy DSA Process Flow

Another aspect of this invention is of is a process selective deposition of a self-assembled monolayer, followed by directed self-assembly of a block copolymer comprising the steps:
ic) using lithography processing to form a chemoepitaxy array of metal and non-metal lines on a substrate,
iic) coating said substrate with a PMMA affinity brush polymer, or an inventive PMMA affinity SAM composition, as described herein.
iiic) baking at a temperature ranging from about 150° C. to about 200° C.,
ivc) rinsing with an organic spin casting solvent the produce a substrate in which only the metal lines have an attached PMMA affinity brush, or a PMMA affinity self-assembled monolayer,
vc) coating with an inventive Neutral affinity SAM composition as described herein,
vic) baking at a temperature ranging from about 150° C. to about 200° C.,
viic) rising with an organic spin casting solvent to produce a substrate in which the metal lines still have an attached PMMA brush, but also have a Neutral affinity self-assembled monolayer attached to the non-metal lines,
viiic) coating the substrate formed in step viic) with a block copolymer solution,
vivc) annealing the block copolymer coating to form directed self-assembled block copolymer L/S pattern.

### Process of Selective Deposition of an Inventive PMMA Affinity and an Inventive PS Affinity Self-Assembled Monolayer as Used in a Chemoepitaxy DSA Process Flow

Another aspect of this invention is of is a process for selective removal of a self-assembled monolayer and directed self-assembly of a polymer comprising the steps;
id) using lithography processing to form a chemoepitaxy array metal and non-metal lines on a substrate
iid) coating with an inventive PS affinity SAM composition as described herein,
iiid) baking at a temperature ranging from about 150° C. to about 200° C.,
ivd) rinsing with an organic spin casting solvent to produce a substrate in which the whole substrate has an attached PS affinity self-assembled monolayer, on both the metal and non-metal lines,
vd) treating with a removal solution, to selectively remove the PS affinity self-assembled monolayer, wherein said removal solution is selected from the group consisting of
   a removal solution with a pH from about 3 to about 5 comprising, water, glycine, hydrogen peroxide solutions and a secondary acid component selected from a dicarboxylic acid, a tricarboxylic acid or an aryl sulfonic acid substituted with an alkyl,
   a removal solution with a pH from about 3 to about 5 consisting of an alcoholic glycolic spin casting solvent, water, and an acid component selected from tricarboxylic acid, having a pKₐ from about 2.9 to about 5, a dicarboxylic acid having a pKₐ from about 3 to about 5 or an alkyl substituted aryl sulfonic acid,
   a removal solution consisting of an alcoholic glycolic spin casting solvent, and a dithiol and
   a removal solution consisting of an alcoholic glycolic spin casting solvent, and an amine,
vid) optionally baking at a temperature ranging from about 100° C. to about 200° C.,
viid) rinsing with an organic spin casting solvent to produce a substrate in which on the whole substrate only has PS affinity self-assembled monolayer on the non-metal lines,
viiid) coating with an inventive Neutral affinity SAM composition as described herein,
ixd) baking at a temperature ranging from about 150° C. to about 200° C.,
xd) rinsing with an organic spin casting solvent to produce a substrate in which the metal lines have an attached Neutral affinity self-assembled monolayer, and the non-metal lines have an attached PS affinity self-assembled monolayer,
xd) coating the substrate formed in step ixd) with a solution of a block copolymer which has polar and non-polar block segments,
xid) annealing the block copolymer coating to form directed self-assembled block copolymer L/S pattern.

### Process of Selective Removal of a PMMA Affinity Self-Assembled Monolayer as Used in a Chemoepitaxy DSA Process Flow

Another aspect of this invention is the process of coating with a PMMA affinity SAM composition on a chemoepitaxy array of metal and non-metal lines and selectively removing deposited SAM on the metal lines, comprising the steps:
ie) using lithography processing to form a chemoepitaxy array of metal and non-metal lines on a substrate
iie) coating a substrate with an inventive PMMA affinity SAM composition as described herein,
iiie) baking at a temperature ranging from about 150° C. to about 200° C.,
ive) rinsing with an organic spin casting solvent to form a PMMA affinity self-assembled monolayer on both the metal lines and non-metal lines,
ve) treating with a removal solution, to selectively remove the PMMA affinity self-assembled monolayer from the metal lines, wherein said removal solution is selected from the group consisting of
   a removal solution with a pH from about 3 to about 5 comprising, water, glycine, hydrogen peroxide solutions and a secondary acid component selected from a dicarboxylic acid, a tricarboxylic acid or an aryl sulfonic acid substituted with an alkyl,
   a removal solution with a pH from about 3 to about 5 consisting of an alcoholic glycolic spin casting solvent, water, and an acid component selected from tricarboxylic acid, having a pKₐ from about 2.9 to about 5, a dicarboxylic acid having a pKₐ from about 3 to about 5 or an alkyl substituted aryl sulfonic acid,
   a removal solution consisting of an alcoholic glycolic spin casting solvent, and a dithiol and
   a removal solution consisting of an alcoholic glycolic spin casting solvent, and an amine,
vie) optionally baking at a temperature ranging from about 100° C. to about 200° C.,
viie) treating with an organic spin casting solvent to remove the cleaved SAM producing a substrate in which only the non-metal lines have attached a PMMA affinity self-assembled monolayer.

### Process of Selective Removal of a PS Affinity Self-Assembled Monolayer

Another aspect of this invention is a process of coating with a PS affinity SAM composition on a chemoepitaxy array of metal and non-metal lines and selectively removing deposited SAM on the metal lines, comprising the steps:
if) using lithography processing to form a chemoepitaxy array of metal and non-metal lines on a substrate
iif) coating a substrate with an inventive PS affinity SAM composition as described herein,
iiif) baking at a temperature ranging from about 150° C. to about 200° C.,
ivf) rinsing with an organic spin casting solvent to form a PS affinity self-assembled monolayer on both the metal lines and non-metal lines,
vf) treating with a removal solution to selectively remove the PS affinity self-assembled monolayer from the metal lines, wherein said removal solution is selected from the group consisting of
   a removal solution with a pH from about 3 to about 5 comprising, water, glycine, hydrogen peroxide solutions and a secondary acid component selected from a dicarboxylic acid, a tricarboxylic acid or an aryl sulfonic acid substituted with an alkyl,
   a removal solution with a pH from about 3 to about 5 consisting of an alcoholic glycolic spin casting solvent, water, and an acid component selected from tricarboxylic acid, having a pKₐ from about 2.9 to about 5, a dicarboxylic acid having a pKₐ from about 3 to about 5 or an alkyl substituted aryl sulfonic acid,
   a removal solution consisting of an alcoholic glycolic spin casting solvent, and a dithiol and
   a removal solution consisting of an alcoholic glycolic spin casting solvent, and an amine,
vif) optionally baking at a temperature ranging from about 100° C. to about 200° C.,
viif) treating with an organic spin casting solvent to remove the cleaved PS affinity self-assembled monolayer from the metal lines producing a substrate in which only the non-metal lines have attached a PS affinity self-assembled monolayer.

### Process of Selective Removal of a Neutral Affinity Self-Assembled Monolayer

Another aspect of this invention is the process of coating with a Neutral affinity SAM composition on a chemoepitaxy array of metal and non-metal lines and selectively removing deposited SAM on the metal lines, comprising the steps:
ig) using lithography processing to form a chemoepitaxy array of metal and non-metal lines on a substrate
iig) coating a substrate with an inventive Neutral affinity SAM composition as described herein,
iiig) baking at a temperature ranging from about 150° C. to about 200° C.,
ivg) rinsing with an organic spin casting solvent to form a Neutral affinity self-assembled monolayer on both the metal lines and non-metal lines,
vg) treating with a removal solution to selectively remove the Neutral affinity self-assembled monolayer from the metal lines, wherein said removal solution is selected from the group consisting of
   a removal solution with a pH from about 3 to about 5 comprising water, glycine, hydrogen peroxide solutions and a secondary acid component selected from a dicarboxylic acid, a tricarboxylic acid or an aryl sulfonic acid substituted with an alkyl,
   a removal solution with a pH from about 3 to about 5 consisting of an alcoholic glycolic spin casting solvent, water, and an acid component selected from tricarboxylic acid, having a pKₐ from about 2.9 to about 5, a dicarboxylic acid having a pKₐ from about 3 to about 5 or an alkyl substituted aryl sulfonic acid,
   a removal solution consisting of an alcoholic glycolic spin casting solvent, and a dithiol and
   a removal solution consisting of an alcoholic glycolic spin casting solvent, and an amine,
vig) optionally baking at a temperature ranging from about 100° C. to about 200° C.,
viig) treating with organic spin casting solvent to remove the cleaved Neutral affinity self-assembled monolayer from the metal lines producing a substrate in which only the non-metal lines have attached a Neutral affinity self-assembled monolayer.

### Solvents for the Processes

In the above inventive processes suitable solvent which are any organic spin casting solvent which is not deleterious to either the substrate itself or to the patterned metal and non-metal lines which may be present on said substrate. It is preferred for some SAM formulation to employ alcoholic glycolic spin casting solvents (e.g. PGME or mixture of PGME and PGMEA). Rinse solution employed to remove any residual unattached SAM precursors from a patterned substrate may be organic spin casting as described herein.

### Removal Solutions

Several different types of remover solutions were employed in the inventive processes described herein to selectively remove the inventive SAM from metallic patterns on substrate and not cleave SAM attached to non-metallic patterns on a substrate, such as silicon oxides, are as follows:
a removal solution with a pH from about 3 to about 5 comprising water, glycine, hydrogen peroxide solutions and a secondary acid component selected from a dicarboxylic acid, a tricarboxylic acid or an aryl sulfonic acid substituted with an alkyl, preferably C-8 to C-14 alkyl,
a removal solution with a pH from about 3 to about 5 consisting of an alcoholic glycolic spin casting solvent, an acid component selected from tricarboxylic acid (preferably citric acid), having a pKₐ from about 2.9 to about 5, a dicarboxylic acid having a pKₐ from about 3 to about 5 or an alkyl substituted aryl sulfonic acid (preferably dodecylbenzenesulfonic acid), and optionally water,
a removal solution consisting of an alcoholic glycolic spin casting solvent, and a dithiol and
a removal solution consisting of an alcoholic glycolic spin casting solvent, and an amine, and further
   These remover solutions useful for selectively removing SAM from a metal pattern are described in more details as follow:

### Aqueous Remover Solution of Glycine, Hydrogen Peroxide and a Secondary Acid Component.

One type of removal solution with a pH from about 3 to about 5 is comprised of water, glycine, hydrogen peroxide, and a secondary acid component selected from a tricarboxylic acid, a dicarboxylic acid or a C-8 to C-14 alkyl substituted arylsulfonic acid.

In one aspect of this removal solution, it comprises about 3 to about 7 wt. % hydrogen peroxide. In another aspect of this embodiment, it comprises from 4 to about 6 wt. %. In yet another aspect of this embodiment if comprises from about 5 wt. %.

In another aspect of this removal solution, it comprises from about 7 to about 12 wt. % glycine. In another aspect of this embodiment if comprises from about 8 to about 11 wt. % glycine. In still another aspect of this embodiment it comprises from about 9 to about 10.5 wt. % glycine. In still another aspect it comprises about 9.8 wt. % glycine.

In another of this removal solution it comprises from about 0.1 to about 2 wt. % of the secondary acid component selected from a tricarboxylic acid, a dicarboxlic acid. In one aspect of this embodiment the secondary acid component is selected from a dicarboxylic or tricarboxylic acid having a pka from about 1.3 to about 5. In another aspect of this embodiment the dicarboxylic acid is succinic acid, glutaric acid, adipic acid malonic or oxalic acid; in one aspect of this embodiment, it is oxalic or adipic acid. In another aspect of this embodiment the tricarboxylic acid is selected from citric acid, isocitric acid and 1,2,3-propanetricarboxylic acid; in another aspect of this embodiment, it is citric acid. In another embodiment the acid is citric acid.

In another embodiment of this removal solution it comprises from about 0.1 to about 2 wt. % of a C-8 to C-14 alkyl substituted aryl sulfonic acid; in another aspect of this embodiment the acid is dodecylbenzenesulfonic acid.

This solution may also optionally contain about 40 to about 60 wt. % of an alcoholic glycolic spin casting solvent which is a 60:30 to 80:20 wt/wt mixture of an alkylene moiety functionalized a hydroxy and an alkyloxy and a alkylene moiety functionalized with both an alkylcarboxyloxy and an alkyloxy. In a specific aspect of this embodiment, it is a mixture of PGMEA and PGME. In another more specific embodiment, it is about a 70:30 mixture of PGME and PGMEA. In all the ranges described herein for the different components of this removal solution, these components are chosen such that their total equals 100 wt. %.

Removal Solutions Consisting of an Alcoholic Glycolic Spin Casting Solvent, and an Acid Component Selected from Tricarboxylic Acid, a Dicarboxylic Acid and an Alkyl Substituted Aryl Sulfonic Acid.

This remover solution which has a pH from about 3 to about 5, comprises about 70 to about 90 wt. % of an alcoholic glycolic spin casting solvent which is a 60:30 to 80:20 wt/wt mixture of an alkylene moiety functionalized a hydroxy and an alkyloxy and an alkylene moiety functionalized with both an alkylcarboxyloxy and an alkyloxy. In a specific aspect of this embodiment, it is a mixture of PGME and PGMEA. In another more specific embodiment, it is about a 70:30 wt:wt mixture of PGME and PGMEA. In one aspect of this removal solution, it comprises from about 10 to about 30 wt. % water. In another of this removal solution it comprises from about 1 to about 2 wt. % of a tricarboxylic acid, having a pKₐ from about 2.9 to about 5, In another aspect of this embodiment the tricarboxylic acid is selected from citric acid, isocitric acid and 1,2,3-propanetricarboxylic acid; in another aspect of this embodiment, it is citric acid. In another embodiment the acid is citric acid. In another of this removal solution it comprises from about 1 to about 2 wt. % of a dicarboxylic acid, having a a pKₐ between about 3 and about 5. In another aspect of this embodiment the dicarboxylic acid is succinic acid, glutaric acid, or adipic acid. In another embodiment of this this removal solution it comprises from about 1 to about 2 wt. % of C-8 to C-14 alkyl substituted aryl sulfonic acid; in another aspect of this embodiment the acid is dodecylbenzenesulfonic acid. In all the ranges described herein for the different components of this removal solution, these components are chosen such that their total equals 100 wt. %.

### Removal Solutions Consisting of an Alcoholic Glycolic Spin Casting Solvent, and a Dithiol.

In one aspect of this removal solution, it comprises about 98 to about 99 wt. % of an alcoholic glycolic spin casting solvent which is a 60/30 to 80/20 wt/wt mixture of an alkylene moiety functionalized with a hydroxy and an alkyloxy and a alkylene moiety functionalized with both an alkylcarboxyloxy and an alkyloxy. In a specific aspect of this embodiment, it is a mixture of PGME and PGMEA. In another more specific embodiment, it is about a 70:30 mixture of PGME and PGMEA. In one aspect of this removal solution, it consists of about 1 to about 2 wt % of a C-3-C6 alkane dithiol. In another aspect of this embodiment, it consists of a butane dithiol selected from propane dithiol thiol, butane dithiol and an pentane dithiol. In all the ranges described herein for the different components of this removal solution, these components are chosen such that their total equals 100 wt. %.

### Removal Solutions Consisting of an Alcoholic Glycolic Spin Casting Solvent, and an Amine.

In one aspect of this removal solution, it comprises about 98 to about 99 wt. % of an alcoholic glycolic spin casting solvent which is a 60/30 to 80/20 wt/wt mixture of an alkylene moiety functionalized a hydroxy and an alkyloxy and an alkylene moiety functionalized with both an alkylcarboxyloxy and an alkyloxy. In a specific aspect of this embodiment, it is a mixture of PGMEA and PGME. In another more specific embodiment, it is about a 70:30 mixture of PGME and PGMEA. In one aspect of this removal solution, it consists from about 1 to about 2 wt % of a C-2 to C-4 trialkylamine. In another aspect of this embodiment, it consists of an amine selected from the group consisting of triethylamine, tripropylamine, and tributylamine. In all the ranges described herein for the different components of this removal solution, these components are chosen such that their total equals 100 wt. %.

### Rinse Solvent to Remove Cleaved SAM

In the processes to selectively remove SAM, described herein, after treatment with the remover solution it is required to rinse with an organic spin casting solvent.

### Examples

### Chemicals

All chemicals unless otherwise indicated were purchased from Sigma Aldrich (3050 Spruce St., St. Louis, MO 63103).

All synthetic experiments were carried out under N₂ atmosphere. Lithographic experiments were carried out as described in the text. The molecular weight of the copolymers was measured with a Gel Permeation Chromatograph. Gel permeation chromatography equipped with 100 Å, 500 Å, 10³ Å, 10⁵ Å and 10⁶ Å μ-ultrastyragel columns

Lithographic Experiments were done using a TEL Clean ACT8 track. SEM pictures were taken with an applied Materials NanoSEM_3D Scanning electron microscope picture are shown at either 1 FOV magnification or 2 FOV magnification (Field of view (FOV)=5 μm).

Etching experiments were done using standard isotropic oxygen etching conditions for self-assembled films block copolymer of methyl methacrylate and styrene.

Unless otherwise indicated Molecular weight measurements (a.k.a. Mₙ polydispersity) were done by Gel permeation chromatography (PSS Inc. Germany) equipped with 100 Å, 500 Å, 10³ Å, 10⁵ Å and 10⁶ Å-ultrastyragel columns using THF solvent as an eluent. Polystyrene polymer standards were used for calibration.

¹H NMR spectra were recorded in CDCl₃ solvent using Bruker Advanced III 400 MHz spectrometer.

The molecular weight of the copolymers was measured with a Gel Permeation Chromatograph. Chemicals, unless otherwise indicated, were obtained from the Sigma-Aldrich Corporation (St. Louis, Missouri).

Lithographic Experiments were done using a TEL Clean ACT8 track. SEM pictures were taken with an applied Materials NanoSEM_3D Scanning electron microscope picture are shown at either 1 FOV magnification or 2 FOV magnification (Field of view (FOV)=5 μm using 1, 2, and 5 FOV).

Etching experiments were done using standard isotropic oxygen etching conditions for self-assembled films block copolymer of methyl methacrylate and styrene.

Unless otherwise indicated Molecular weight measurements (a.k.a. Mₙ polydispersity) were done by Gel permeation chromatography (PSS Inc. Germany) equipped with 100 Å, 500 Å, 103 Å, 10⁵ Å and 10⁶ Å-ultrastyragel columns using THF solvent as an eluent. Polystyrene polymer standards were used for calibration.

### Synthesis of Polymeric Materials for Testing

### Polymer Synthesis Example 1: Synthesis of PS-co-PMMA Neutral Layer Material

250-ml flask equipped with a temperature controller, heating mantle and magnetic stirrer were set up. 26.04 grams (0.25 moles) of styrene, 24.03 grams (0.24 moles) of methyl methacrylate, 1.42 grams (0.10 moles) of glycidyl methacrylate, 0.41 grams (0.0025 moles) of Azobisisobutyronitrile (AIBN) initiator and 100 grams of anisole were added to the flask. The stirrer was turned on and set up at about 400 rpm. The reaction solution was then degassed by vigorously bubbling nitrogen through the solution for about 30 minutes at room temperature. After 30 minutes of degassing the heating mantle was turned on and the temperature controller was set at 70° C., and the stirred reaction mixture was maintained at this temperature for 20 hours. After this time the heating mantle was turned off and the reaction solution was allowed to cool down to about 40° C. Then the reaction mixture was poured into 1.5 L of isopropanol stirred with a mechanical stirring during the addition. During this addition, the polymer was precipitated out. The precipitated polymer was collected by filtration. The collected polymer was dried in vacuum oven at 40° C. About 36 grams of the polymer was obtained. This dried polymer was dissolved in 300 grams of THF and then filtered through a 0.2 um nylon filter. The filtered solution was then precipitated again into a stirred solution of 1.5 L methanol, the precipitated polymer collected and dried as before under vacuum at 40° C. In this manner, 26 grams (50% yields) of the polymer was obtained after dry. The polymer had an M_{W} of about 36k and a polydispersity (PDI) of 1.5.

### Polymer Synthesis Example 2: Synthesis of P(S-b-MMA) (78K-b-39K)

Styrene and methyl methacrylate monomers were distilled in the presence of dehydrating agents into calibrated ampules and stored under N₂. Liquids were transferred into the reactor either via ampule or using stainless steel cannula under N₂. Into a dry 1 L round bottom reactor equipped with side arms for connecting ampules, magnetic stir bar, nitrogen/vacuum three-way septum adapter, was added 700 mL dry tetrahydrofuran. The temperature of the reactor was reduced to −78° C. using dry ice-acetone bath. Then, after titrating the impurities, 0.2 mL (1.4 M solution) of sec-butyllithium was added into the reactor. Then 20 g (0.192 moles) of styrene was added from ampule into the reactor under fast stirring. The reaction solution turned into yellow-orange and the reaction was stirred over 30 minutes. Subsequently, 0.06 g (0.0003 moles) of 1,1′-diphenylethylene (DPE) in 2.5 mL of dry toluene was added via ampule into the reactor. The orange color of the reaction mixture turned into dark brick-red indicating conversion of styryl lithium active centers to delocalized DPE adduct carbanion. After 2 min of stirring, a small amount (2 mL) of the reaction mixture was withdrawn for PS block molecular weight analysis. Then methyl methacrylate (9.98 g, 0.0998 moles) was added via ampule. The reaction was terminated after 30 min with 1 mL of degassed methanol. The block copolymer was recovered by precipitation in excess isopropanol (5 times of the polymer solution) containing 10% water, filtered, and dried at 55° C. for 12 h under vacuum giving 28 g of P(S-b-MMA) (94% yield) consisting of 66 mol. % of polystyrene block and 34 mol. % of polymethylmethacrylate block.

Gel permeation chromatography equipped with 100 Å, 500 Å, 103 Å, 10′ A and 10⁶ Å-ultrastyragel columns showed that the 1^{st} P(SDPE) block had Mₙ (GPC)=64,622 g/mol and M_{W}/Mₙ=1.02 with respect to PS calibration standards. The diblock copolymer molecular weight obtained from GPC is M_{n,PS-b-PMMA}=107,150 g/mol and M_{W}/Mₙ=1.01.

### Polymer Synthesis Example 3 Synthesis of P(S-b-MMA) (45k-b-51k)

P(S-b-MMA) (45K-b-51K) was synthesized using the same procedure as described in example 2. To achieve target Mₙ and compositions of PS and PMMA block, the amount of initiator and monomer quantities were changed. Briefly, 20 g (0.192 moles) of styrene was polymerized with 0.32 mL (1.4M solution) of sec-butyllithium. Then 0.095 g (0.0005 moles) of 1,1′-diphenylethylene (DPE) in 2.5 mL of dry toluene was added via ampule into the reactor. The orange color of the reaction mixture turned into dark brick-red indicating conversion of styryl lithium active centers to delocalized DPE adduct carbanion. After 2 min of stirring, a small amount (2 mL) of the reaction mixture was withdrawn for PS block molecular weight analysis. Then methyl methacrylate (22.85 g, 0.23 moles) was added via ampule. The reaction was terminated after 30 min with 1 mL of degassed methanol. The block copolymer was recovered by precipitation in excess isopropanol (5 times of the polymer solution) containing 10% water, filtered, and dried at 55° C. for 12 h under vacuum giving 40 g of P(S-b-MMA) (94% yield) consisting of 46.9 mol. % of polystyrene block and 53.1 mol. % of polymethylmethacrylate block.

Gel permeation chromatography equipped with 100 Å, 500 Å, 103 Å, 10′ A and 10⁶ Å-ultrastyragel columns showed that the 1^{st} P(SDPE) block had Mₙ (GPC)=45,048 g/mol and M_{W}/M=1.04 with respect to PS calibration standards. The diblock copolymer molecular weight obtained from GPC is M_{n,PS-b-PMMA}=88,348 g/mol and M_{W}/Mₙ=1.02.

### Synthesis of Novel SAM Precursor Molecules for Testing

### Section A: This Section Describes Synthesis of Multi-Hydroxyl Alkyl SAM

### Synthesis of SAM Precursors:

In general, all the multiple hydroxyl group SAM precursors were made using Williamson's etherification reaction of multi-hydroxy aromatic or aliphatic core with corresponding bromo-alkyl-alcohol, or bromo-alkyl-olefin. Reaction with aromatic core is single step whereas, aliphatic core glycerol involves multistep synthesis. Molecules are named Mx where M refers multi-tether, x refers number.
1) Protocol 1: Starting aromatic diols were reacted with alpha-bromo-alkyl-omega alcohol under basic conditions at reflux condition in 2-butanone (hydroxyl functional molecules). Solid compound were purified via recrystallization from 2-butanone.
2) Protocol 2: Starting aromatic diols were reacted with alpha bromo alkyl diethyl phosphonate under basic conditions at reflux condition in 2-butanone (diethylphosphonate functional molecules)
3) Protocol 3: Diethyl phosphonate functional groups were made using two step approach. In first step starting hydroxyl functional molecules these were converted into bromo derivatives, then these bromo derivatives were reacted with excess triethyl phosphite. After removal of any excess triethyl phosphite, the desired compounds were obtained.
4) Protocol 4: This protocol involves multi-step synthesis using protected hydroxyl derivatives or olefin conversion into alcohol.

### Synthesis of (M-1):

Scheme 1 shows the general synthesis pathway which led to M-1. To a round bottom flask provided with magnetic stir bar, condenser, and nitrogen blanket was added 9.2 g. of 4,4′,4″-(ethane-1,1,1-trityl)triphenol g (0.03 moles), 28.2 g (0.112) of 11-bromoundecan-1-ol, 28 g. (0.202 moles) potassium carbonate, and 3 g of potassium iodide (0.0018 moles) and 500 mL methyl ethyl ketone which was stirred at a temperature of 90° C. for 24 hours. Reaction mass was passed over short silica bed. Rotovaped, and column chromatographed using 100% ethyl acetate as mobile phase. Removal of ethyl acetate gave yellowish viscous liquid (18.0 g M-1 73.5 yield). The compound was purified using column chromatography with eluent 570:30 ethyl acetate and hexane mixture. FIG. 5 shows the ¹H NMR of this this material (M-1).

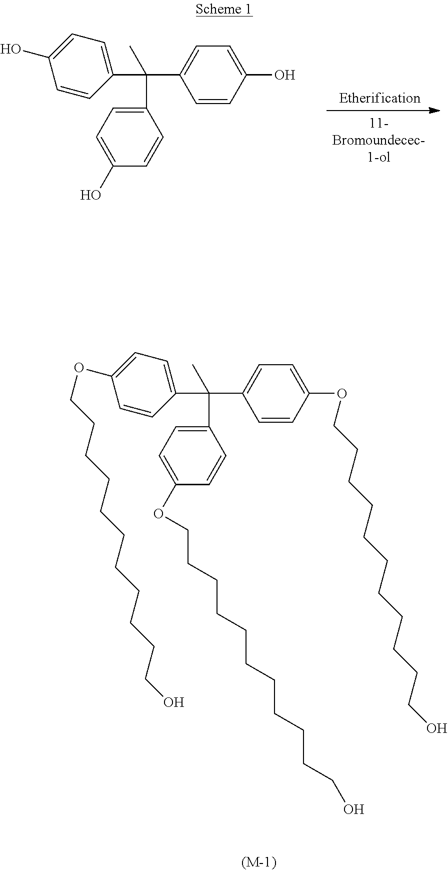

### Synthesis of M-2

Scheme 2 shows the overall synthetic pathway which was used to make M-2 which comprised of steps I to IV as follow:

Step-I: To a three neck round bottom flask provided with magnetic stir bar, condenser, dropping funnel and nitrogen blanket was added triethylamine, 10-undecenol, and 500 mL dichloromethane (DCM), Mesyl chloride 108 g was transferred to dropping funnel. After cooling the RBF with ice bath mesyl chloride added dropwise maintaining temperature below 10° C. Stirred at low temperature for 1h, then at RT for few hours. After aqueous workup and removal of dichloromethane 201 g of mesyl protected 10-undecenol was obtained. FIG. 6 shows the ¹H NMR of this product, undec-10-en-1-yl methanesulfonate (undecenyl methanesulfonate).
Step-IT: To a three-neck round bottom flask provided with dropping funnel, condenser with dean-stark-apparatus and thermometer, under nitrogen blanket was added powdered 96 g KOH, 92.3 g Solketal ((2,2-dimethyl-1,3-dioxolan-4-yl)methanol), and 600 mL toluene. The flask was heated using hot plate at 150° C., at which toluene starts to condense in dean-stark apparatus. After stirring the viscous solution for 30 min (no visible water removal seen) 110 g undecenyl mesylate was added over 20 min, immediate reaction was seen (rapid condensation of toluene). After 30 min the reaction mass became not able of being stirred, then the reaction mixture was cooled to RT and it was transferred to separatory flask, neutralized with con. HCl, and 400 mL of heptane was added. The organic layer was washed with brine, and 500 mL of distilled water (DIW). The organic layer dried over sodium sulfate and filtered, evaporated on roto-evaporated to give a pale-yellow-brownish liquid 114 g. This liquid was subjected to deprotection of ketal group using Amberlyst™-15 in methanol under reflux for 3 h. The pure product isolated using column chromatography with 1:1 ethyl acetate and heptane. The yield was 58 g of 3-(undec-10-en-1-yloxy)propane-1,2-diol (undecenyl ether glycerin) whose ¹H NMR is shown in FIG. 6.
Step-III: To a three-neck round bottom flask provided with dropping funnel, condenser with dean-stark-apparatus and thermometer, under nitrogen blanket were added powdered 90.7 g KOH, 26.4 g, the step-II product (undecenyl ether glycerin), and 630 mL toluene. This reaction mixture was heated using a metal hot plate at 150° C., until toluene started to condense in dean-stark apparatus. After stirring the resultant viscous solution for 30 min (in which no visible water removal was seen), 56 g undecenyl mesylate was added over 20 min, an immediate reaction was seen (rapid condensation of toluene). After 30 min, the reaction mixture was cooled to RT and transferred to separatory flask and neutralized with conc. HCl. This mixture was extracted with 400 mL of heptane. The resultant, organic layer was washed with brine, and 500 mL of DIW. The organic layer dried over sodium sulfate and filtered, evaporated on a roto-evaporated to give a pale-yellow-brownish liquid. (Yield=54 g). This crude intermediate was passed over silica column and eluted with heptane to obtain 45 g of desired product 11-((1,3-bis(undec-10-en-1-yloxy)propan-2-yl)oxy)undec-1-ene (undecyl tris-ether glycerol) whose ¹H NMR is shown in FIG. 7.
Step IV: To a two-neck round bottom flask provided condenser and nitrogen blanket, a rubber septum inlet, was added 40 g of step-III product, and 250 mL THF. At RT, quickly 100 mL of BH₃-THF (1M) was added. Immediately, gelation was observed, then the flask was heated with a hotplate at 80° C. for 30 min. Then 70 mL of hydrogen peroxide was added which dissolved the gel, after 5 min, 13 g KOH dissolved in 70 mL water was added and the flask was heated for further 30 min. The resultant water/THF layer was separated in the flask, and to the total mixture was added to 400 mL DIW with HCl to neutralize KOH. A sticky white glue-like product was obtained, which was dissolved in DCM and water layer was separated and dried over sodium sulfate upon removal of DCM gave white semi-solid was obtained. (Yield=37 g). This product shows multiple TLC spots and contains mixture of pure tris-hydroxy derivative and unreacted olefin groups. Purification was carried out using hot heptane. Mixed Olefin/hydroxyl containing compound dissolves in heptane and tris-hydroxy compound is insoluble in heptane. Yield 19 g, % hydroxy functionality=˜92%). FIG. 8 shows the ¹H NMR spectrum of this product.

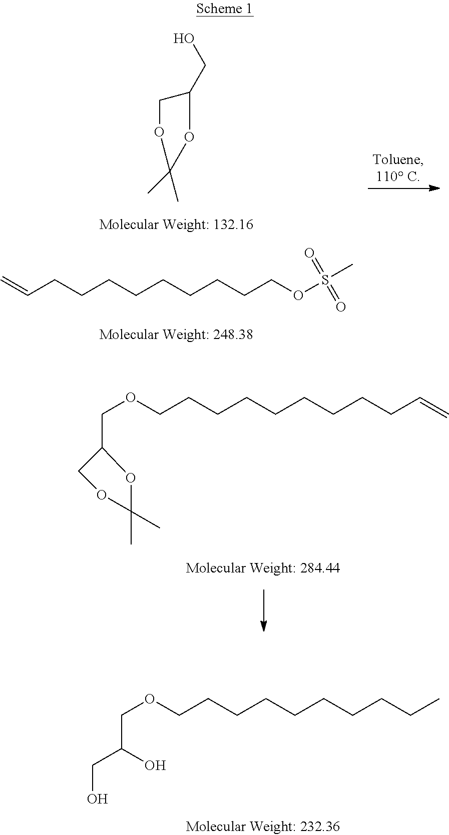

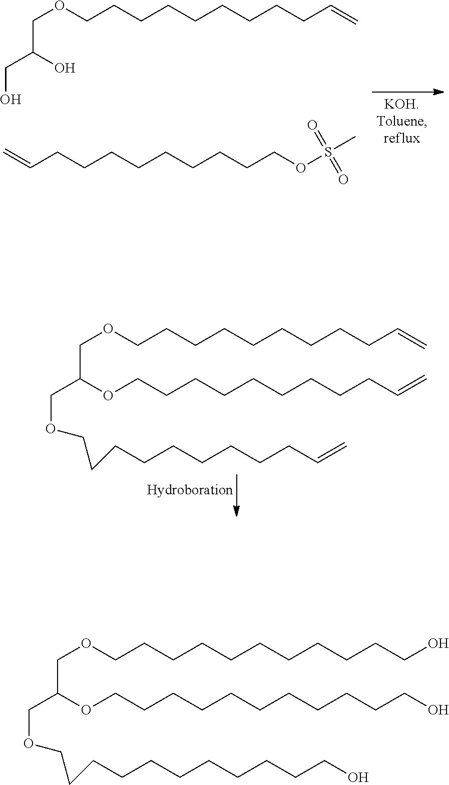

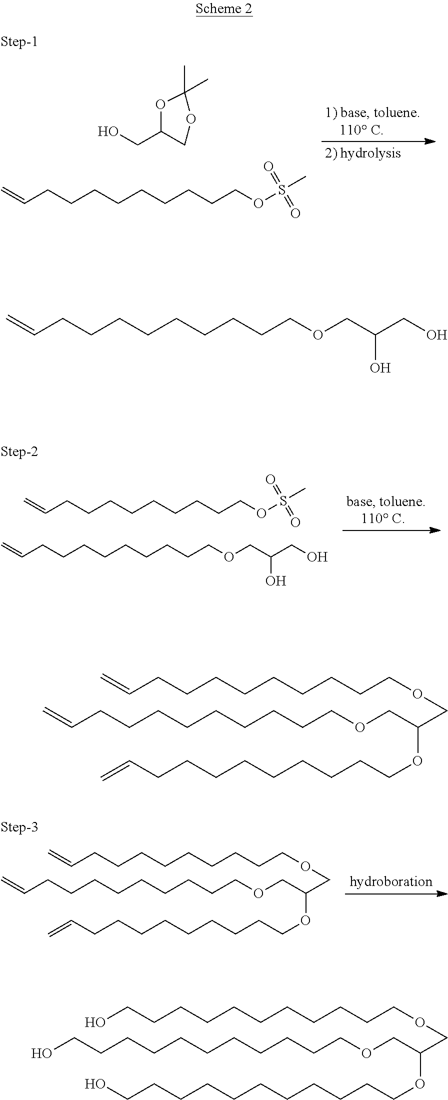

### Synthesis of M-3

Scheme 3 shows the synthetic pathway used to make M-3 which was made as follows: To a three neck RBF provided with a N₂ inlet, condenser and temperature probe, the following reagents were added: 0.2024 moles of 11-bromo 1-undecanol, 0.0713 moles of pyrogallol, 0.4573 moles of potassium carbonate, 0.030 moles of potassium iodide, and 250 mL of acetone. The flask was heated until acetone started refluxing and kept under reflux temperature for 72 h. The reaction mixture was then cooled to RT and filtered and washed with excess acetone ˜500 mL. The acetone roto-evaporated to give a yellowish crude solid. (TLC showed three spots, and absence of starting pyrogallol). The tris ether M-3 was recovered by recrystallization from hot acetonitrile. The yield was 29.6 g. FIG. 10 shows its ¹H NMR spectrum of this material M-3 (11,11′,11″-(benzene-1,2,3-triyltris(oxy))tris(undecan-1-ol).

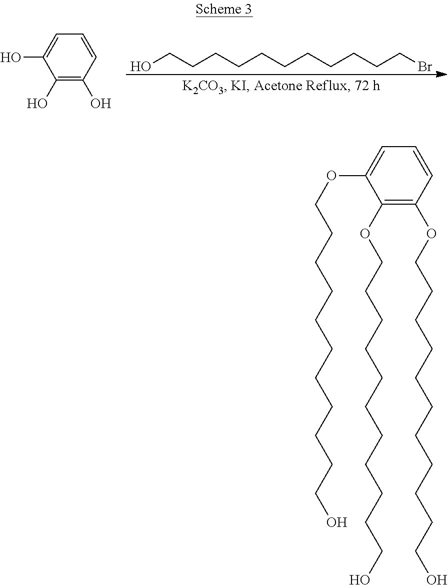 [SMILES: Oc1cccc(O)c1O ; OCCCCCCCCCCCBr ; OCCCCCCCCCCCOc1cccc(OCCCCCCCCCCCO)c1OCCCCCCCCCCCO]

### Synthesis of M-4

Scheme 4 shows the synthetic pathway which was used to make M-4 as follows: To a three neck RBF provided with a N₂ inlet, condenser and temperature probe, the following reagents were added: 8.25 g (0.075 moles) 1,2 dihydroxy benzene, 38.9 g (0.155 moles) 11-bromo 1-undecanol, 100 g (0.723 moles) of potassium carbonate, 9 g (0.0542 moles) of potassium iodide, and 420 mL of methyl ethyl ketone (MEK). Flask was heated until MEK starts refluxing and kept under reflux temperature for 48 h. Hot solution was filtered over short silica column and washed with excess MEK ˜200 mL. Solution was kept at −20° C. overnight and crystallized, and a white solid was isolated by filtration. Yield 26.0 g, (76.5%), FIG. 11 shows the ¹H NMR spectra of this material M-4 (11,11′-(1,2-phenylenebis(oxy))bis(undecan-1-ol).

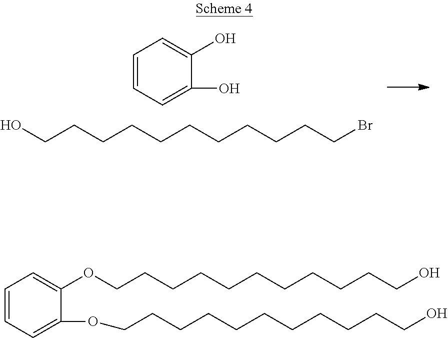

### Synthesis of M-5

Structure (M-5) shows the structure of this material which was made as follows: To a three neck RBF provided with a N₂ inlet, condenser and temperature probe, all the reagents were added: 27.6 g (0.1722 moles) 2,3 dihydroxy naphthalene, 96.2 g (0.3829 moles) 11-bromo 1-undecanol, 190.6 g (1.3799 moles) of potassium carbonate, 19.9 g (0.1198 moles) of potassium iodide, and 1500 mL of methyl ethyl ketone (MEK). The flask was heated until MEK starts refluxing and kept under reflux temperature for 72 h. The hot solution was filtered over short silica column and washed with excess MEK ˜300 mL. The solution was kept at −20° C. overnight and crystallized white solid was isolated by filtration. Yield 65 g, 75.4%). See structure (M-5)(11,11′-(naphthalene-2,3-diylbis(oxy))bis(undecan-1-ol)) and the ¹H NMR spectrum in FIG. 12.

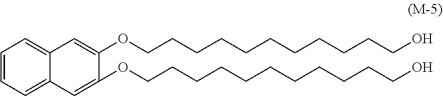 [SMILES: OCCCCCCCCCCCOc1cc2ccccc2cc1OCCCCCCCCCCCO]

### Synthesis of M-6

The structure of M-6 is shown in structure (M-6), which was made as follows: To a three neck RBF provided with a N₂ inlet, condenser and temperature probe, all the reagents were added: 5.6 g (0.0.03796 moles) 1,8 dihydroxy naphthalene, 22.3 g (0.08871 moles) 11-bromo 1-undecanol, 55.7 g (0.400 moles) of potassium carbonate, 6.8 g (0.0496 moles) of potassium iodide, and 400 mL of methyl ethyl ketone (MEK). The flask was heated until MEK starts refluxing and kept under reflux temperature for 24 h. The hot solution was filtered over short silica column and washed with excess MEK ˜100 mL. Solution was kept at −20° C. overnight and crystallized white solid was isolated by filtration. Yield 15 g, (85.7%). FIG. 13 shows the ¹H NMR for M-6 (11,11′-(naphthalene-1,8-diylbis(oxy))bis(undecan-1-ol)).

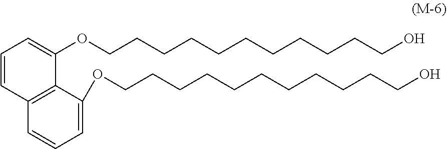 [SMILES: OCCCCCCCCCCCOc1cccc2cccc(OCCCCCCCCCCCO)c12]

### Synthesis of M-7

The structure of M-7 is shown in structure (M-7), which was made as follow using a Grignard cross coupling of 1,2 dibromobenzene and 11-bromo-undecyl alcohol THP ether (2-((11-bromoundecyl)oxy)tetrahydro-2H-pyrane) in presence of Pd catalyst. First a Grignard agent was prepared from 11-bromo-undecylTHP and Magnesium in THF, which was added to mixture of 1,2 dibromo benzene and a Ni catalyst [1,2-Bis(diphenylphosphino)ethane]dichloronickel(II) at RT. This mixture was refluxed for 24h, cooled to RT, neutralized with dilute HCL and extracted in heptane. After solvent removal, the resultant yellowish liquid was hydrolyzed with conc. HCl in methanol under reflux. After cooling to RT, this mixture was precipitate in DIW, the solid filtered and washed with acetone to obtain M-7 as white solid (11,11′-(1,2-phenylenebis(oxy))bis(undecan-1-ol). FIG. 14 shows the ¹H NMR spectrum of M-7.

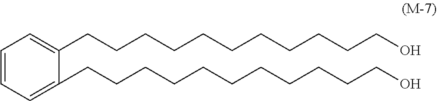 [SMILES: OCCCCCCCCCCCc1ccccc1CCCCCCCCCCCO]

### Synthesis of M-8:

The structure of M-8 is shown in structure (M-8). To a three neck RBF provided with a N₂ inlet, condenser and temperature probe, were added: 15.7 g (0.09625 moles) 4-terbutyl catechol, 50.4 g (0.1990 moles) 11-bromo 1-undecanol, 159.6 (1.152 moles) of potassium carbonate, 13.8 g (0.083 moles) of potassium iodide, and 800 mL of methyl ethyl ketone (MEK). This flask was heated until the MEK starts refluxing and kept under reflux temperature for 14 h. The hot solution was filtered over short silica column and washed with excess MEK ˜100 mL. This solution was kept at −20° C. overnight and crystallized white solid was isolated by filtration. Yield 28.8 g (60.0%) of M-8 (11,11′-((4-(tert-butyl)-1,2-phenylene)bis(oxy))bis(undecan-1-ol)). FIG. 15 shows the ¹H NMR of M-8.

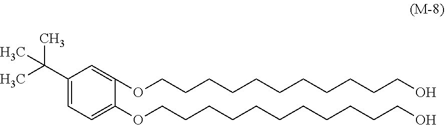 [SMILES: CC(C)(C)c1ccc(OCCCCCCCCCCCO)c(OCCCCCCCCCCCO)c1]

### Synthesis of M-9

The structure of M-9 is shown in structure (M-9). To a three neck RBF provided with a N₂ inlet, condenser and temperature probe, all the reagents were added: 1.0 g (0.00908 moles) catechol, 6.0 g (0.01867 moles) 16-bromo 1-hexadecanol, 15.0 (0.1082 moles) of potassium carbonate, 1.50 g (0.00904 moles) of potassium iodide, and 100 mL of methyl ethyl ketone (MEK). Flask was heated until MEK starts refluxing and kept under reflux temperature for 32 h. The hot solution was filtered over short silica column and washed with excess MEK ˜100 mL. The solution was kept at −20° C. overnight and crystallized white solid was isolated by filtration. Yield 4.2 g (78.3%) of M-9 (16,16′-(1,2-phenylenebis(oxy))bis(hexadecan-1-ol). FIG. 16 shows the ¹H NMR spectra of M-9.

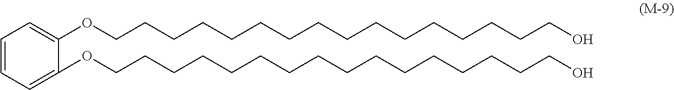 [SMILES: OCCCCCCCCCCCCCCCCOc1ccccc1OCCCCCCCCCCCCCCCCO]

### Synthesis of M-10

The structure of M-10 is shown in structure (M-10). To a three neck RBF provided with a N₂ inlet, condenser and temperature probe, all the reagents were added: 12.5 (0.09250 moles) cyno-catechol, 50.4 (0.1990 moles) 11-bromo 1-undecanol, 110.0 (0.796 moles) of potassium carbonate, 9.2 g (0.0554 moles) of potassium iodide, and 750 mL of methyl ethyl ketone (MEK). Flask was heated until MEK starts refluxing and kept under reflux temperature for 20 h. The hot solution was filtered over short silica column and washed with excess MEK ˜500 mL. The solution was kept at −20° C. overnight and crystallized white solid was isolated by filtration. Yield 36 g (81.8%) of M-10 (3,4-bis((11-hydroxyundecyl)oxy)benzonitrile). FIG. 17 shows the ¹H NMR spectra of M-10.

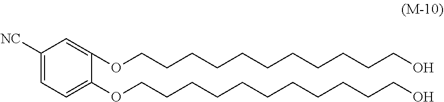 [SMILES: N#Cc1ccc(OCCCCCCCCCCCO)c(OCCCCCCCCCCCO)c1]

### Synthesis of M-11

The structure of M-11 is shown in structure (M-11). To a three neck RBF provided with a N₂ inlet, condenser and temperature probe, were added: 20.5 g (0.1586 moles) pyrogallol, 35.5 (0.1522 moles) 11-bromo 1-undecene, 71.7 (0.5188 moles) of potassium carbonate, 7.0 g (0.0421 moles) of potassium iodide, and 800 mL of methyl ethyl ketone (MEK). The flask was heated until MEK started to reflux, and the reaction mixture was kept under reflux temperature for 30 h. The hot solution was filtered over a short silica column and washed with excess MEK ˜500 mL. The solvent was evaporated on a roto-evaporator to obtain brownish liquid (˜30 g), which contained both mono and di ethers. Mono and Di-ethers which were separated using column chromatography with eluent containing 10% ethyl acetate and 90% heptane. The yield was: Monoether: 1.6 g, and Di-ether: 27.7 g. The di-ether was subjected to etherification with 11-bromo 1-undecanol: To a three neck RBF provided with a N₂ inlet, condenser and temperature probe were added: 24.7 g (0.0555 moles) pyrogallol diether, 13.5 (0.1522 moles) 11-bromo 1-undecanol, 40.0 (0.28943 moles) of potassium carbonate, 4.5 g (0.0271 moles) of potassium iodide, and 400 mL of methyl ethyl ketone (MEK). The flask was heated until MEK started to reflux and was kept under reflux temperature for 48 h. The hot solution was filtered over short silica column and washed with excess MEK ˜500 mL. The solvent was removed on a roto-evaporator, to obtain a brownish liquid (˜30 g), which contained both mono and di ethers. Mono and Di-ethers were separated using column chromatography with eluent 10% ethyl acetate: 90% heptane. The yield was 31 g of M-11(11-(2,6-bis(undec-10-en-1-yloxy)phenoxy)undecan-1-ol). FIG. 18 shows the ¹H NMR spectra of M-11.

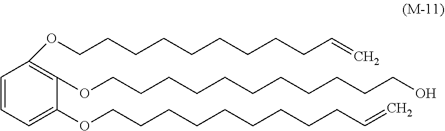 [SMILES: C=CCCCCCCCCCOc1cccc(OCCCCCCCCCC=C)c1OCCCCCCCCCCCO]

### Synthesis of M-12

The structure of M-12 is shown in structure (M-12). To a three neck RBF provided with a N₂ inlet, condenser and temperature probe, were added: 10.0 (0.0908 moles) catechol, 50.0 (0.2108 moles) 10-bromo 1-decanol, 92.0 (0.0.6654 moles) of potassium carbonate, 12 g (0.07228 moles) of potassium iodide, and 1000 mL of methyl ethyl ketone (MEK). The flask was heated until MEK starts refluxing and kept under reflux temperature for 48 h. The hot solution was filtered over short silica column and washed with excess MEK ˜500 mL. The solution was kept at −20° C. overnight and crystallized white solid was isolated by filtration. Yield 30.7 g (80.0%) of M-12 (10,10′-(1,2-phenylenebis(oxy))bis(decan-1-ol). FIG. 18 shows the ¹H NMR spectra of M-12.

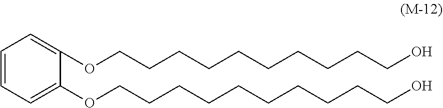 [SMILES: OCCCCCCCCCCOc1ccccc1OCCCCCCCCCCO]

### Synthesis of M-13, M-14 and M-15

M-13, M-14, and M-15 M15 were synthesized using synthetic pathway as shown in scheme 5. In a first step, 11,11′-(1,2-phenylenebis(oxy))bis(undecan-1-ol) was converted into the bromide. This bromide was subsequently converted according to scheme 3 to either converted to the diethyl phosphonate (M-13) (tetraethyl ((1,2-phenylenebis(oxy))bis(undecane-11,1-diyl))bis(phosphonate)) (yield >90%), sulfide (M-15)(11,11′-(1,2-phenylenebis(oxy))bis(undecane-1-thiol)) (yield >90%), or the azide -(M-14)(1,2-bis((11azidoundecyl)oxy)benzene) (yield >90%), FIG. 20, FIG. 21, FIG. 22 and FIG. 23, respectively show the 1H NMR spectra of the bromide precursor in Scheme 5, 11,11′-(1,2-phenylenebis(oxy))bis(undecane-1-thiol) (M-13), 1,2-bis((11-azidoundecyl)oxy)benzene (M-14), tetraethyl ((1,2-phenylenebis(oxy))bis(undecane-11,1-diyl))bis(phosphonate) (M-15).

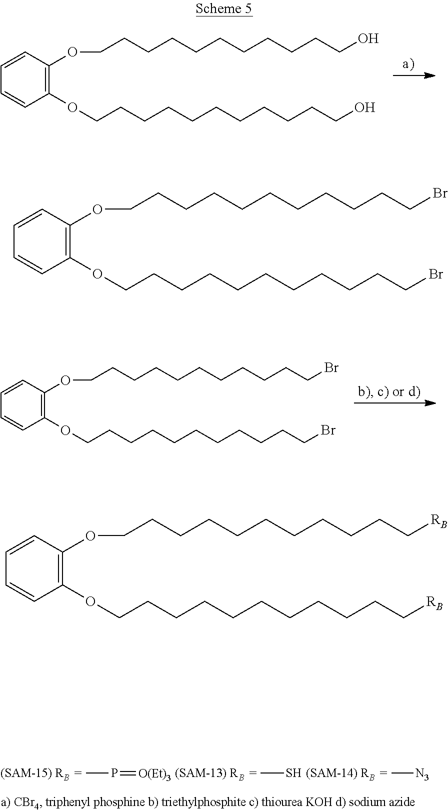

### Synthesis of M-16

The structure of M-16 (11,11′-((4-methyl-1,2-phenylene)bis(oxy))bis(undecan-1-ol)) is shown in scheme 6, where protocol 1 was used to make this material in 70% yield. FIG. 24 shows the ¹H NMR spectrum of M-16

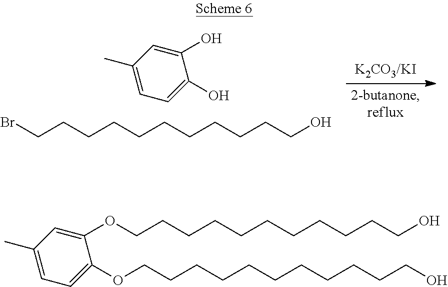

The structure of M-17: (methyl 3,4-bis((11-hydroxyundecyl)oxy)benzoate) is shown in scheme 7, which was synthesized using protocol 1 in 65% yield. FIG. 25 shows the ¹H NMR spectrum of M-17

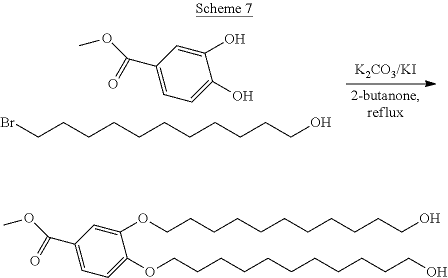

The structure of M-18 (11,11′-((4-nitro-1,2-phenylene)bis(oxy))bis(undecan-1-ol)) is shown in scheme 8, which was synthesized using protocol 1 in 70% yield. FIG. 26 shows the ¹H NMR spectrum of M-18

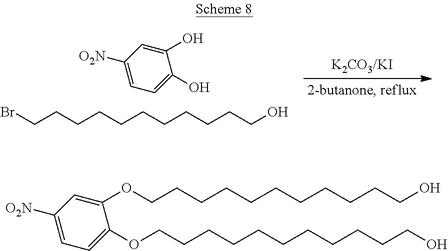

The structure of M-19 (dimethyl 4,5-bis((11-hydroxyundecyl)oxy)phthalate) is shown in scheme 9, which was synthesized using protocol 1 in 60% yield. FIG. 27 shows the ¹H NMR spectrum of M-19

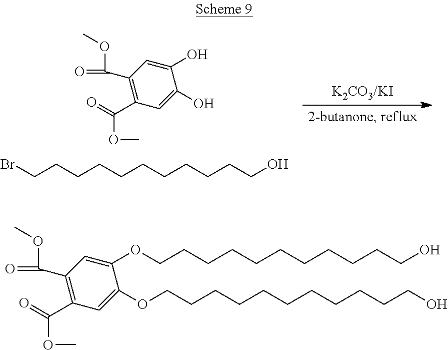

The structure of M-20 (3,4-bis((11-hydroxyundecyl)oxy)benzonitrile) is shown in scheme 10, using reduction of nitro group using tin-chloride in ethanol, which by this procedure was synthesized in 80% yield. FIG. 28 shows the ¹H NMR spectrum of M-20

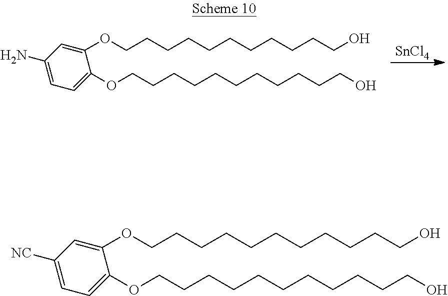

### Synthesis of M-21:

The synthesis of M-21 ((E)-11,11′-((4-(hex-1-en-1-yl)-1,2-phenylene)bis(oxy))bis(undecan-1-ol)) was done by Protocol 3 which is shown in scheme 11, in a three-step protocol from starting dihydroxy benzaldehyde and alpha-bromo-alkyl hydroxyl groups protected with tetrahydropyran. This step was followed by Wittig reaction with butyl triphenyl phenyl bromide salt, and a subsequent hydrolysis of THP groups using Amberlyst®-15 to give M-21 in 40% yield. FIG. 29 shows the ¹H NMR spectrum of THP protected M-21 and FIG. 30 shows the ¹H NMR spectrum of M-21.

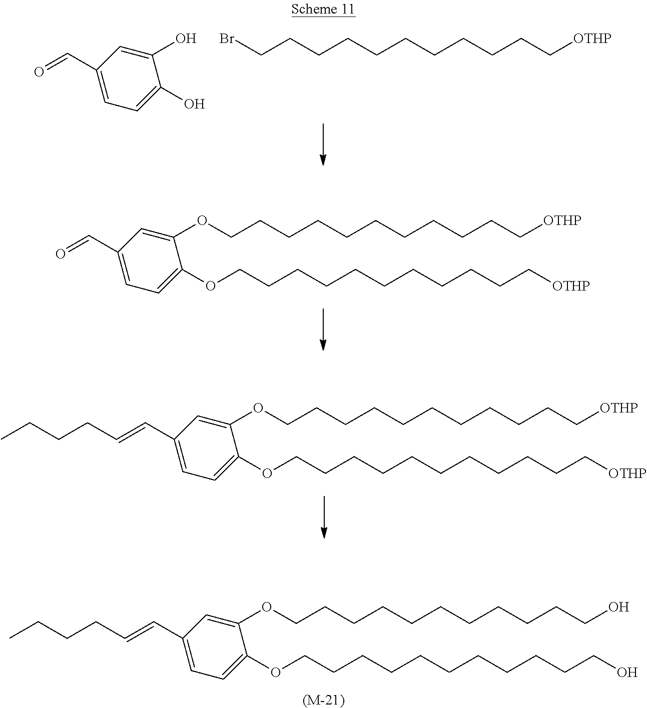

### Synthesis of M-22

The synthesis of M-22 (tetraethyl (((4-cyano-1,2-phenylene)bis(oxy))bis(undecane-11,1-diyl))bis(phosphonate)) was done by Protocol 2 which is shown in scheme 12, which gave M-22 in >95% yield. FIG. 31 and FIG. 32 show, respectively the ¹H NMR spectrum and the ³¹P NMR spectrum of M-22.

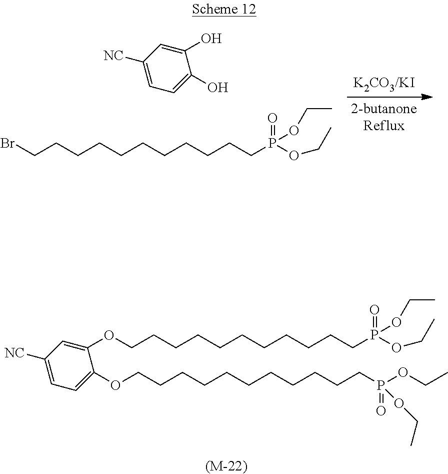

### Synthesis of M-23

Scheme 13 shows the synthesis pathway for making of M-23 (tetraethyl (((4-acetyl-1,2-phenylene)bis(oxy))bis(undecane-11,1-diyl))bis(phosphonate)) in >95% yield. FIG. 33 and FIG. 34 shows respectively the ¹H and ³¹P NMR spectra for M-23.

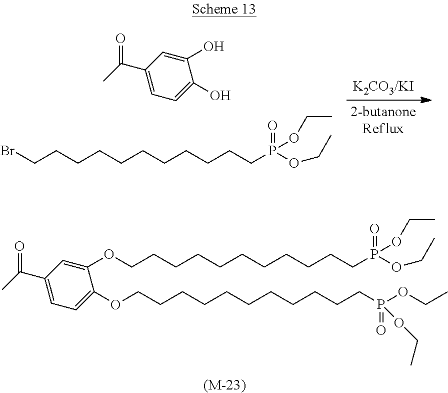

### Synthesis of M-24:

Synthesized using protocol-2 Scheme 14 shows the synthesis pathway for making of M-23 (dimethyl 4,5-bis((11-(diethoxyphosphoryl)undecyl)oxy)phthalate) in >95% yield. FIG. 35 and FIG. 36 shows respectively the ¹H and ³¹P NMR spectra for M-24.

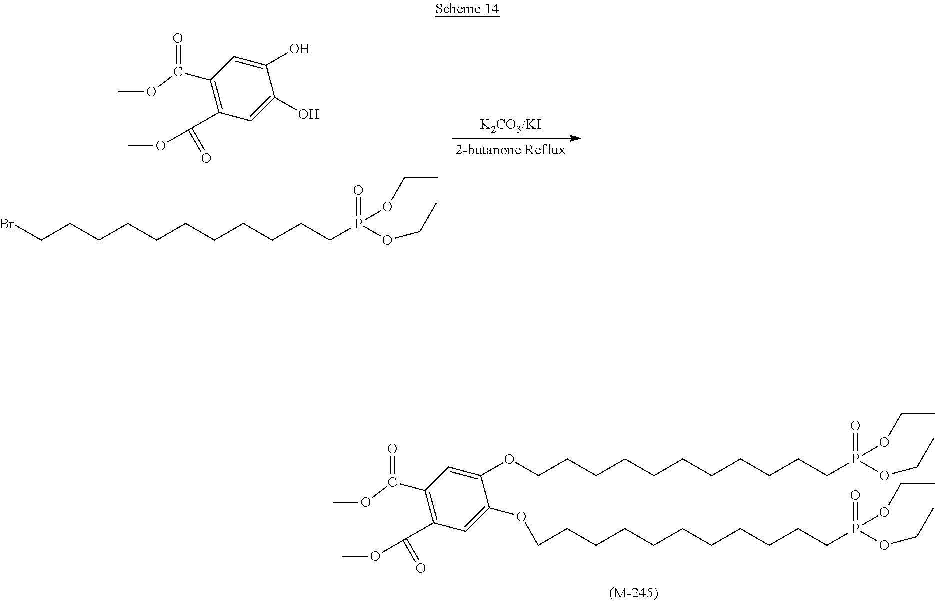

### Synthesis of M-25:

M-25 (tetraethyl (((4-nitro-1,2-phenylene)bis(oxy))bis(undecane-11,1-diyl))bis(phosphonate)) was synthesized using protocol-2.as shown in scheme 15 and was obtained in >95% yield. FIG. 37 shows the 1H NMR spectra of M-25.

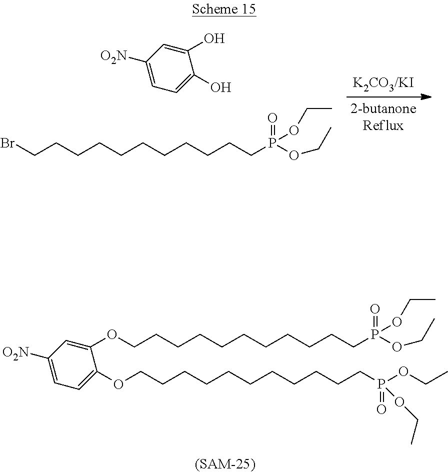

### Section-B: Processing of Materials

This section describes the processing of the inventive compounds to form self-assembled monolayer (SAM) on a SiOx substrate and the testing of the interaction of this SAM.

### X-Ray Photoelectron Spectroscopy (XPS) Measurement

These were done using K-alpha from Thermo-Fisher. Experiments were runs using pass energy 50 eVe, step size, 0.100 eV, dwell time 50 ms and 10 scans per element.

### Water Contact Angle (WCA) Measurement

These were done with dynamic contact angle measurement tool from Kruss at room temperature. 4 microliter droplets of denoised water was used. Reported values are average of measurements of 5-6 spots per 1 xl inch coupons.

### Preparation of PS-b-PMMA BCP Solution for SAM Testing.

The polymer Synthesis Example 3 was dissolved in 70/30 PMMEA to form a 0.4 wt. % solution which was filtered was filtered with a 0.2 μm PTFE.

### Si/SiOx Wafer

Six- and eight-inch wafers were obtained from Silicon Valley Microelectronics (SVM).

### X-Ray Reflectivity (XRR) Measurements.

These measurements were done with at Darmstadt Technical University, Germany.

### Ellipsometry

Ellipsometry thickness measurement was done using J. A. Woollam. SAM FTs were measured using a single layer model with RI of organic layer 1.45.

### Selective Wetting of Block Copolymer

SAM precursors with the prefix “M-,” were individually dissolved in PGMEA and respective formulation denoted with the prefix “SML-,” SAM assembly was formed by spin-coating 1% PGMEA SML solutions at 2000-300 RPM, followed by bake at 125-200° C. for 5-30 min under nitrogen or air. After baking, excess SAM precursor removed by rinse with PGMEA and after air blow drying were used for further analysis. Film thickness of the SAM were measured using ellipsometry. Tables 1 and Table 2 give a summary the properties of the of the self-assembled monolayers formed by some of the SAM precursors on Si/SiO₂ wafers and also reports the self-assembly behavior, after annealing, of a representative block copolymer (PS-b-PMMA) which contains both a polar and a non-polar block segments (Table 1). Table 1 shows the results of the selected wetting testing experiments in which WCA, carbon and atomic %. The inventive SAM materials with variable tail groups, and hydroxyl anchor groups were coated on blanket Si/SiOx wafer as described herein to form a self-assembled layer on the Si/SiOx wafer whose WCA was measured and XPS measurements of atomic % of carbon were done to confirm that the self-assembled layer had formed on the substrate as reported in Table 1. Then, each Si/SiOx substrate with these different SAM coatings were individually coated with the BCP (PS-b-PMMA) solution to obtain a 50 nm (1 Lo) film thickness BCP coating. This BCP coating was then annealed 250° C. for 20 min, and the surface morphology of the block copolymer film was checked by SEM and water contact angle of the block copolymer was measures as reported in Table 1. The non-polar tail groups preferably wetted the PS block of BCP and polar-tail groups wetted the PMMA block of BCP, as shown in Table 1 where SEM Morphological examination the block copolymer film shows that both polar and non-polar tail group give a flat morphology indicative of a preference respectively to either the polar PMMA block and non-polar PS block segments of this block copolymer because either the non-polar PS block segment or the polar PMMA block segment strongly interacted with respectively a non-polar SAM or a polar SAM adopting an block copolymer segment parallel to the SAM layer. For the short alkyl tail group, methyl, and especially the H tail group it was observed that the block copolymer upon annealing formed a fingerprint pattern indicative that the block copolymer segment showed not preference for either the SAM substrate and adopted an orientation perpendicular to the substrate. Table 1 confirms that preferred wetting of BCP was obtained via proper choice of tail groups. Thus, when a prepattern was modified with either a non-polar SAM or polar-SAM this enabled respectively parallel alignment of either the non-polar PS polymer block segment or the polar PMMA block segment of PS-b-PMMA resulting in the flat morphology observed by SEM. Conversely, when the SAM resulting from percussors with H or methyl as a tail, were used, the polymer block segments polar or non-polar of PS-b-PMMA did not show a strong affinity adopted an orientation perpendicular to the SAM surface resulting in the formation of the fingerprint pattern which was observed by SEM. As such the polar and non-polar SAM, substituted respectively with polar or non-polar tail groups, can be used in a chemoepitaxy DSA process if patterned to form, respectively, pinning polar or non-polar pinning regions on a patterned substrate. Additionally, the SAM material with H tail groups was used as a neutral layer in DSA lithographic processing such as chemoepitaxy, in some instances the methyl tail SAM material which showed partial neutral layer formation in our testing may also be useful as a neutral layer on other substrates where it may manifest full neutral layer characteristics.

TABLE 1
Effect of SAM tail groups wettability towards PS-b-PMMA

| Compound | | | | | BCP WCA | |
|---|---|---|---|---|---|---|
| M-X | | | SAM | | (°) | |
| (Solution | | | WCA | XPS of SAM | BCP/SAM/ | Morphology |
| number) | Polarity | Tail group | (°) | (C atomic %) | SiOx | (SEM) |
| M-8 | Non- | -tert-Bu | 82.5 | 21.7 | 88.0 | Flat |
| (SML-8) | polar | | | | | |
| M-16 | Non- | —Me | 79.7 | 30.6 | 84.8 | Partial |
| (SML-16) | polar | | | | | Fingerprint |
| | | | | | | Partial Flat |
| M-12 | Neutral | —H | 76.7 | 29.6 | 77.2 | Fingerprint |
| (SML-12) | | | | | | |
| M-10 | Polar | —CN | 63.0 | 26.6 | 67.9 | Flat |
| (SML-10) | | | | | | |
| M-17 | Polar | —CO2Me | 66.3 | 30.5 | 67.7 | Flat |
| (SML-17) | | | | | | |
| M-18 | Polar | NO₂ | 62.5 | 30.6 | 67.4 | Flat |
| (SML-18) | | | | | | |
| M-19 | Polar | —(CO2Me)₂ | 66.9 | 28.5 | 67.5 | Flat |
| (SML-19) | | | | | | |
| M-20 | Polar | NH₂ | 63.4 | 29.3 | 67.7 | Flat |
| (SML-20) | | | | | | |

TABLE 2
Solubility and selective SAM formulations

| | | | | |
|---|---|---|---|---|
| Entry | Anchor | Solubility | WCA | FT |
| (Formulation) | groups | PGMEA | (°) | (nm) |

SAM for dielectric surface

| | | | | |
|---|---|---|---|---|
| M-1 (SML-1) | hydroxyl | Moderate | 80 | 1.8 |
| M-2 (SML-2) | hydroxyl | Moderate | 73 | 1.5 |
| M-3 (SML-3) | hydroxyl | Moderate | 78 | 1.8 |
| M-4 (SML-4) | hydroxyl | Moderate | 78 | 1.7 |
| M-5 (SML-5) | hydroxyl | Moderate | 78 | 1.8 |
| M-6 (SML-6) | hydroxyl | Moderate | 79 | 1.9 |
| M-7 (SML-7) | hydroxyl | Moderate | 83 | 1.5 |
| M-8 (SML-8) | hydroxyl | Moderate | 95 | 1.5 |
| M-9 ((SML-9) | hydroxyl | Moderate | 78 | 2.2 |
| M-10 (SML-10) | hydroxyl | Moderate | 62 | 1.7 |
| M-11 (SML-11) | hydroxyl | Good | 91 | 1.6 |
| M-12 (SML-12) | hydroxyl | Good | 78 | 1.5 |
| M-16 (SML-16) | hydroxyl | Good | 90 | 1.6 |

SAM for metal surface

| | | | | |
|---|---|---|---|---|
| M-13 (SML-13) | thiol | Good | 64 | n/a |
| M-14 (SML-14) | azide | Good | 71 | n/a |
| M-15 (SML-15) | Diethyl | Good | 61 | n/a |
| | phosphonate | | | |

FIGS. 39 and 40 show XRR curves which were measured for few selective SML molecules (SML-8, SML-3 and SML-6) and the comparative material octadecyl trichlorosilane (ODTS-Cl₃). The calculated values obtained from these XRR curve are tabulated in Table 3. As seen from the table, the values of number of molecules per nm² depends on the number of alky chains in a SAM molecule. These electron density values showed very well densely packed SAM molecules on the substrate surface.

TABLE 3
XRR characterization of SAM on SiOx

| | Density | Thickness | ρₑ | Area/molecule | # Molecules/ |
|---|---|---|---|---|---|
| SAM | (g/ml) | (nm) | (e/Å³) | (Å²) | nm² |
| SML-3 | 1.11 | 1.80 | 0.381 | 51.6 | 2.0 |
| SML-5 | 1.07 | 1.93 | 0.360 | 39.7 | 2.6 |
| SML-6 | 1.05 | 1.94 | 0.355 | 40.0 | 2.6 |
| SML-12 | 1.05 | 1.79 | 0.341 | 40.4 | 2.5 |
| SML-2 | 1.08 | 1.52 | 0.350 | 63.2 | 1.6 |
| OTDS | 1.22 | 2.35 | 0.395 | 19.7 | 5.0 |

### Selective SAM Assembly on Dielectric Surface and Passivation Properties:

It was found that at high temperature (>150° C.) SAM's with hydroxyl head groups reacted on silanol Si surfaces and on native metal oxides. For obtaining a dielectric selective SAM, selective removal of SAM from metal lines was required. Metal-O—C— bonds are weak compared to —Si—O—C— bond and susceptible to both acidic and basic reagents. Moreover, the thermal stability of these Metal-O—C— bonds is much lower than Si—O-alkyl bonds. Therefore, in order to selectively deposit dielectric-SAM, metal SAM was removed using formulations containing organic acids, oxidizer, and the chelator, glycine (FIG. 41), Table 5, 6, 7, 8), organic bases (Table 10), and dithiol solutions (2 wt. % butane dithiol in 70:30 PGME: PGMEA) (Table 9), and thermal cleavage (Table 9). All above method showed very negligible effect SAM on SiOx (FIG. 41-44). Additionally, also tested were removal solution which were comprised citric acid and 70/30 PGME/PGMEA and these solutions also did not remove the SAM having Si—O-alkyl bonds. Therefore dielectric-SAM on SiOx substrates remained stable. After SAM were selectively deposited on dielectric, its passivation properties were analyzed against atomic layer deposition of hafnium oxide. Selective deposition of 7-8 nm HfOx on W vs SiOx was achieved (FIG. 45). Tabulated data in Tables 5 to 8 showed that these different remover solutions are selectively removing SAM from metal surface, while showing no effect of SAM on SiOx surface. 1) Table-5 shows composition of glycine/hydrogen peroxide is removing SAM from metal surface but no effect on SiOx SAM. 2) Table 6 shows XPS data for SAM on SiOx vs SAM on metal, negligible C at % shows DBSA selectively removed SAM from metal surface. 3) Table 7 shows the effect of DBSA on metal surface via X-SEM. It shows FT of metal layer is unaffected via DBSA rinse. 4) Table-8 show XPS data indicating SAM is removed from metal surface as evident from negligible Cat % on metal vs no change on SAM on SiOx.

### Selective SAM Assembly on Metal Surface:

M-13, M-14 and M-15 were dissolved in PGEMA at 1 wt. % concentration to make formulation SML-13, SML-14, and SML-15, respectively. Spin-coated at RPM 1500 and baked at 170° C./5 min and unreacted SAM precursor removed by rinse with excess PGMEA. SAM were analyzed using measuring water contact angle (Table 4) and XPS normalized C at % (Figure: 40). Selective deposition on tungsten and copper vs dielectric silanol was obtained via preferred reaction of metal/metal oxide with dithiol, azide, and diethyl phosphonate functional groups.

### Selective SAM Removal on Metal Surface:

The water contact angle information in Table 4 shows that self-assembled monolayers were deposited on W, Cu and Si using SML-13, SML-14 and SML-15.

Table 4 shows the pH of composition of Glycine removal solution in water which contained oxidizers and stabilizers which were used to selectively metal SAM in the presence of non-metal SAM formed with SML-12.

Table 6 shows using XPS data shows the removal effect of a 2 wt. % dodecyl benzene DBSA solution in PGME: PGMEA 70:30 on the SAM's formed from SML-13, SML-14 and SML-15 on different substrates. This data showed that this solution was effective in removing these SAM from either Cu and W, and that this removal was selective because the high carbon count on the SAM formed on Si samples indicated that the SAM was not cleaved on this non-metallic substrate.

Table 7 shows SEM morphology of SAM on metal substrates after DBSA rinse with 2 wt. % dodecylbenzene sulfonic acid in 70:30 PGME: PGMEA followed by a rinse for 2 min with PGMEA, and N₂ blow dry. This SEM morphology showed that this acid is mild and only remove SAM without leaching of the metal layer.

Table 8 shows using thickness, XPS and water contact angle data the effect that effect that a remover solution of 2 wt. % dodecyl benzene (DBSA) in PGME:PGMEA 70:30 had on the removal of SAM formed from SML-12 and SML5 on W, Cu and Si. This showed that in the case of the SAM on Cu and W the SAM is selectively remove from these metal but retained on Si and that that this acid solution is mild and only removed SAM without leaching or corrosion of the metal layer. In this experiment the rinse with the remover for 2 min was followed by a 2 min PGMEA, and N₂ blow dry

Table 5 shows the performance of a citric acid-based remover formulation containing 1 wt. % citric dissolved in a 50:50 wt:wt solution of water and PGME:PGMEA 70:30. This solution was used to rinse the SAM for 2 mins followed by a rinse for 2 min with PGMEA, and an N₂ blow dry. This data showed that the SAM was selectively removed from the metal substrate while remaining intact on Si.

Table 10 shows the effect the butane dithiol had on SAM (M-12) removal. The solution was a 1 wt. % solution in 70:30 PGME: PGMEA. The SAM substrate were rinsed with this solution for 2 mins and then further annealed at higher temperature. Changes to WCA and XPS Cat % on metal surface vs SiOx indicates SAMs selectively removed form metal surface

Table 11 shows the effect base, triethylamine had on SAM (M-12) removal. The solution was a 1 wt. % solution in 70:30 PGME: PGMEA. The SAM substrate were rinsed with this solution for 2 mins and then rinsed for 2 min with PGME: PGMEA 70:30. Changes to WCA and XPS Cat % on metal surface vs SiOx indicates SAMs selectively removed form metal surface

Table 12 shows the effect of thermal treatment to affect selective removal of SAM from the metals Cu and W versus the SAM on Si. This data showed that thermal annealing at higher temperature selectively breaks the SAM bonding to metal surface and removed with rinse with PGME: PGMEA 70:30 mixture.

These solution had a pH range from about 3 to about 5.

Aqueous citric acid solution in EBR 70/30 (70/30 PGME/PGMEA) the above-described Example: Rinsed 2 min 50:50 50% citric acid (aq)/EBR 70/30, rinse 2 min PGMEA, N₂ blow dry (Table-9).

TABLE 6
Selective SAM deposition on metal surface

| | Water contact angle | | |
|---|---|---|---|
| | W | Cu | Si |
| Blank | 60 | 71 | <10 |
| SML-13 | 74 | 64 | 55 |
| SML-14 | 68 | 71 | 45 |
| SML-15 | 63 | 61 | 43 |

TABLE 7
pH of composition of Glycine containing removal solution in water used
for cleaving metal SAM which contains oxidizers and stabilizers

| | | Oxalic | Malonic | | Glycine | | | SiOx | Metal |
|---|---|---|---|---|---|---|---|---|---|
| | H₂O₂ | Acid | Acid | | Acid | | | substrate | Substrate |
| Entry | wt. % | wt | wt. % | DBSA | wt. % | pH | SAM | results | (Results |
| 1 | 5 | 0.05 | | 0.1 | 9.8 | 4.7 | M-12 | No removal | Removal |
| 2 | 5 | 0.10 | | 0.1 | 9.8 | 4.62 | M-12 | No removal | Removal |
| 3 | 5 | 0.15 | | 0.1 | 9.8 | 4.53 | M-12 | No removal | Removal |
| 4 | 5 | 0.05 | | 1.0 | 9.8 | 4.49 | M-12 | No removal | Removal |
| 5 | 5 | 0.10 | | 1.0 | 9.8 | 4.38 | M-12 | No removal | Removal |
| 6 | 5 | 0.15 | | 1.0 | 9.8 | 4.32 | M-12 | No removal | Removal |
| 7 | 5 | | 0.05 | 0.1 | 9.8 | 4.94 | M-12 | No removal | Removal |
| 8 | 5 | | 0.10 | 0.1 | 9.8 | 4.71 | M-12 | No removal | Removal |
| 9 | 5 | | 0.15 | 0.1 | 9.8 | 4.55 | M-12 | No removal | Removal |
| 10 | 5 | | 0.05 | 1.0 | 9.8 | 4.46 | M-12 | No removal | Removal |
| 11 | 5 | | 0.10 | 1.0 | 9.8 | 4.3 | M-12 | No removal | Removal |
| 12 | 5 | | 0.15 | 1.0 | 9.8 | 4.19 | M-12 | No removal | Removal |
| 13 | 5 | | | 0.03 | 9.8 | 5.55 | M-12 | No removal | Removal |
| 14 | 5 | | | 0.06 | 9.8 | 5.31 | M-12 | No removal | Removal |
| 15 | 5 | | | 0.09 | 9.8 | 5.23 | M-12 | No removal | Removal |
| 16 | 5 | | | 0.15 | 9.8 | 5.13 | M-12 | No removal | Removal |
| 17 | 5 | | | 1.50 | 9.8 | 4.51 | M-12 | No removal | Removal |

TABLE 8
DBSA effect on SAM on different substrates

| | XPS normalized C at % | | |
|---|---|---|---|
| Entry (after rinse with DBSA) | Cu | W | Si |
| SML-12 | 1 | 1 | 72 |
| SML-5 | 4 | 2 | 78 |
| SML-7 | 5 | 0 | 86 |

Table 9 (SEM Morphology of SAM on Metal Substrates after DBSA Rinse with 2 wt. % Dodecylbenzene Sulfonic Acid in 70:30 PGME: PGMEA) is Shown in FIG. 47

TABLE 10
Effect of DBSA on SAM removal

| | Film | | XPS |
|---|---|---|---|
| | Thickness | | Normalized |
| Entry | (nm) | WCA | C at % |
| Cu-blank | 352 | 65 | n/a |
| Cu-SML-12/rinse 2% DBSA | 348 | 69 | 1.3 |
| Cu-SML-5/rinse 2% DBSA | 351 | 70 | 4.2 |
| W-blank | 60 | 15 | n/a |
| W-SML-12/rinse 2% DBSA | 61 | 33 | 1.1 |
| W-SML-5/rinse 2% DBSA | 63 | 31 | 1.9 |
| Si-blank | NA | 15 | n/a |
| SiSML-12/rinse 2% DBSA | NA | 79 | 25.1 |
| Si-SML-5/rinse 2% DBSA | NA | 78 | 32.3 |

TABLE 11
Citric acid-based formulation for selective SAM removal

| | Film Thickness | | XPS Normalized |
|---|---|---|---|
| Entry | (nm) | WCA | C at % |
| W SML-16 | n/a | 60.9 | ~0 |
| W SML-16/rinse | n/a | 40.3 | ~0 |
| Si SML-16 | n/a | 77.8 | ~0 |
| Si- SML-16/rinse | n/a | 80.6 | ~0 |

TABLE 12
Effect of butane dithiol on SAM (M-12) removal

| | | | | XPS normalized | | |
|---|---|---|---|---|---|---|
| | WCA | | | C at % | | |
| Entry | W | Cu | Si | W | Cu | Si |
| blank | 8.1 | 40.7 | 12.6 | n/a | n/a | n/a |
| Substrate with SAM | 63.7 | 74.9 | 80.4 | 19 | 26 | 28 |
| dithiol no bake | 34.2 | 40.7 | 77.9 | 2 | 10 | 32 |
| dithiol +110° C./10 min | 37.6 | 40.0 | 77.7 | 3 | 6 | 31 |
| bake | | | | | | |
| dithiol +150° C./5 min bake | 6.7 | 1.3 | 76.9 | 1 | 0 | 32 |
| dithiol +200° C./5 min bake | 0.2 | 6.3 | 80.0 | 3 | 0 | 31 |

TABLE 13
Effect of base, triethylamine on SAM (M-12) removal

| | | | | XPS normalized | | |
|---|---|---|---|---|---|---|
| | WCA | | | C at % | | |
| Entry | W | Cu | Si | W | Cu | Si |
| no additional rinse | 63.7 | 74.9 | 80.4 | 19 | 26 | 28 |
| amine no bake | 26.2 | 72.2 | 78.7 | 6 | 16 | 32 |
| amine + 110° C./10M | 26.4 | 72.7 | 77.3 | 0 | 15 | 32 |
| bake | | | | | | |
| amine + 150° C./5M bake | 24.9 | 56.1 | 79.1 | 0 | 8 | 34 |
| amine + 200° C./5M bake | 19.7 | 15.5 | 76.9 | 3 | 0 | 31 |

TABLE 14
Thermal method of SAM (M-12) removal

| | WCA | | | XPS normalized C at % | | |
|---|---|---|---|---|---|---|
| Entry | W | Cu | Si | W | Cu | Si |
| SAM no second bake | 63.7 | 69.9 | 80.4 | 19 | 26 | 28 |
| 250° C. second bake | 18.5 | 48.9 | 78.5 | 1 | 3 | 22 |
| 300° C. second bake | 11.1 | 46.2 | 71.4 | 3 | 2 | 13 |
| 350° C. second bake | 6.5 | 33.9 | 28.1 | 0 | 0 | 0 |

1. A composition comprising
a compound of structure (I), and an organic spin casting solvent, wherein
in said compound of structure (I), A is a core moiety which is selected from structure (Ia), (Ib), (Ic) and (Id), to which is attached through X, a direct valence bond or a divalent linking group, m number of linear alkylene moieties of chain length n, wherein n ranges from 8 to 12 and where each said linear alkylene moiety has a terminal B reactive moiety, and further wherein * designates possible attachment point of said linear alkylene moieties in each structure, wherein structure (Ia), and (Ib), have 2 or 3 attachment points (m=2 or 3) which are on adjacent carbons, structure (Ic) has 2 attachment point (m=2), structure (Id), has three attachment point (m=3),
B is selected from —OH, —CH═CH₂, —O—(P=O)(OR)₂, —O—(P=O)(OR)Rs, —N₃, and —SH, wherein R and Rs are independently selected from a C-1 to C-8 alkyl,
R₁ₐ and R_{1b} are independently selected from H a C-1 to C-4 alkyl, a C-1 to C-4 alkyloxy, a C-3 to C-6, methyl(carbonyloxyalkyl) (—CH₂—(C=O)—O-alkyl), a C-2 to C-5 carbonyloxyalkyl (—(C=O)—O-alkyl), nitro (NO₂), NH₂, and CN,
R_{1c}, R_{1d}, R₁ₑ, R_{1f} and R_{1g} are independently selected from H a C-1 to C-4 alkyl, a C-1 to C-4 alkyloxy, C-2 to C-5 carbonyloxyalkyl (—(C=O)—O-alkyl), a C-3 to C-6 methylcarbonyloxyalkyl (—CH₂—(C=O)—O-alkyl), and CN, and
wherein for the core moieties (Ia), (Ib), (Ic) the divalent linking group is selected from the group consisting the group consisting of oxy (—O—), oxycarbonyl (—O—C(═O)—), carbonyloxy(—C(═O)—O—), carbonyl (—(C=O)—), sulfinyl (—(S(═O))—), and sulfone (—S(═O)₂—) and for core moieties (Id), the divalent linking group is selected from the group consisting of oxy (—O—), oxycarbonyl (—O—C(═O)—), carbonyloxy(—C(═O)—O—), ethyne 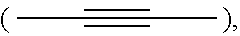
1,4-phenylene(-Ph-), 1,4-phenyleneoxy(-Ph-O—), 1,4-oxyphenylene(—O-Ph-), carbonyl (—(C=O)—), sulfinyl (—(S(═O))—), sulfone (—S(═O)₂—), 1,2-ethene(-CH═CH—), 1,1-ethene (—C(=CH₂)—), and methylene (—CH₂—), and
where said organic spin casting solvent is selected from the group consisting of a glycol ether derivative, a glycol ether ester derivative, a carboxylate, carboxylates of dibasic acids, a dicarboxylate of s glycol, a ketone ester, a ketone ether derivative, ketone alcohol derivative, lactones, an amide derivative, and mixtures thereof, and further wherein said compound of structure (I) comprises from about 0.5 wt. % to about 3.00 wt. % of said composition,

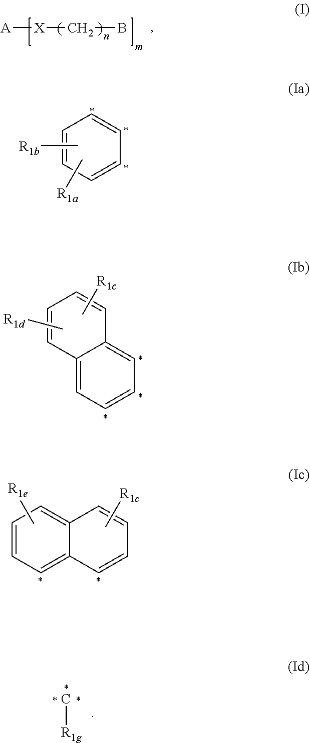

2. The composition according to claim 1, wherein said organic spin casting solvent is selected from the group consisting of o a glycol ether derivative, a glycol ether ester derivative, and mixtures thereof.

3. The composition according to claim 1, wherein said compound of structure (I) is selected from the group consisting of 11,11′,11″-((ethane-1,1,1-triyltris(benzene-4,1-diyl))tris(oxy))tris(undecan-1-ol) (M-1), 11,11′,11″-(propane-1,2,3-triyltris(oxy))tris(undecan-1-ol) (M-2), (11,11′,11″-(benzene-1,2,3-triyltris(oxy))tris(undecan-1-ol).(M-3), (11,11′-(1,2-phenylenebis(oxy))bis(undecan-1-ol). (M-4), 11,11′-(naphthalene-2,3-diylbis(oxy))bis(undecan-1-ol(M-5), 11,11′-(naphthalene-1,8-diylbis(oxy))bis(undecan-1-ol (M-6), 11,11′-(1,2-phenylenebis(oxy))bis(undecan-1-ol (M-7), 11,11′-((4-(tert-butyl)-1,2-phenylene)bis(oxy))bis(undecan-1-ol) (M-8), 1,2-phenylenebis(oxy))bis(hexadecan-1-ol (M-9), 3,4-bis((11-hydroxyundecyl)oxy)benzonitrile (M-10), 11-(2,6-bis(undec-10-en-1-yloxy)phenoxy)undecan-1-ol (M-11), 10,10′-(1,2-phenylenebis(oxy))bis(decan-1-ol (M-12), 11,11′-(1,2-phenylenebis(oxy))bis(undecane-1-thiol) (M-13), 1,2-bis((11-azidoundecyl)oxy)benzene (M-14), tetraethyl ((1,2-phenylenebis(oxy))bis(undecane-11,1-diyl))bis(phosphonate) (M-15), 11,11′-((4-methyl-1,2-phenylene)bis(oxy))bis(undecan-1-ol) (M-16), methyl 3,4-bis((11-hydroxyundecyl)oxy)benzoate (M-17), 11,11′-((4-nitro-1,2-phenylene)bis(oxy))bis(undecan-1-ol) (M-18), dimethyl 4,5-bis((11-hydroxyundecyl)oxy)phthalate (M-19), 3,4-bis((11-hydroxyundecyl)oxy)benzonitrile (M-20), and (E)-11,11′-((4-(hex-1-en-1-yl)-1,2-phenylene)bis(oxy))bis(undecan-1-ol) (M-21).

4.-18. (canceled)

19. The composition of claim 1, wherein structure (Ia) has structure (Ia-1) and m=2, where * designates the position of the two attachment points,

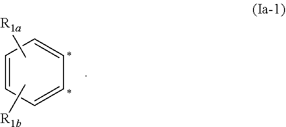

20. The composition of claim 19, wherein said compound of structure (Ia-1) is one where X, is selected from the group consisting of a direct valence bond or oxy (—O—).

21. The composition of claim 1, wherein said compound of structure (I) is selected from the group consisting of (11,11′-(1,2-phenylenebis(oxy))bis(undecan-1-ol). (M-4), 11,11′-(1,2-phenylenebis(oxy))bis(undecan-1-ol (M-7), 1,2-phenylenebis(oxy))bis(hexadecan-1-ol (M-9), 3,4-bis((11-hydroxyundecyl)oxy)benzonitrile (M-10), 10,10′-(1,2-phenylenebis(oxy))bis(decan-1-ol (M-12), 11,11′-((4-methyl-1,2-phenylene)bis(oxy))bis(undecan-1-ol) (M-16), methyl 3,4-bis((11-hydroxyundecyl)oxy)benzoate (M-17), 11,11′-((4-nitro-1,2-phenylene)bis(oxy))bis(undecan-1-ol) (M-18), dimethyl 4,5-bis((11-hydroxyundecyl)oxy)phthalate (M-19), 3,4-bis((11-hydroxyundecyl)oxy)benzonitrile (M-20), and (E)-11,11′-((4-(hex-1-en-1-yl)-1,2-phenylene)bis(oxy))bis(undecan-1-ol) (M-21).

22. The composition of claim 1, wherein in said compound of structure (I), said core moiety A, has structure (Ia), with m=3,

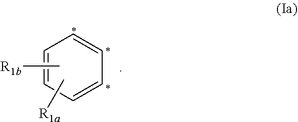

23. The composition of claim 22, wherein said compound of structure (Ia) is one is one where B is selected from the group consisting of —OH, —CH═CH₂ and —SH.

24. The composition of claim 23, wherein said compound of structure (Ia) is one where X, is selected from the group consisting of a direct valence bond and a oxy (—O—).

25. The composition of claim 24, wherein said compound of structure (Ia), is either (11,11,11″-(benzene-1,2,3-triyltris(oxy))tris(undecan-1-ol) (M-3) or 11-(2,6-bis(undec-10-en-1-yloxy)phenoxy)undecan-1-ol (M-11).

26.-33. (canceled)

34. The composition of claim 1, wherein said compound of structure (I) in one wherein said core moiety A, either has structure (Ib), where m is 3 or structure (Ib-1), where * designates the attachment points and B is selected from the group consisting of —OH, —CH═CH₂ and —SH,

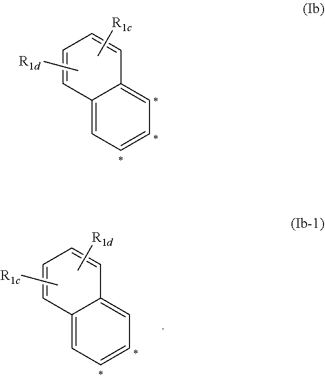

35. The composition of claim 34, wherein X is selected from the group consisting of a direct valence bond; and oxy (—O—).

36.-47. (canceled)

48. The composition of claim 1, wherein in said compound of structure (I), said core moiety A, has structure (Ic), where * designates the attachment points and B is selected from the group consisting of —OH, —CH═CH₂ and —SH,

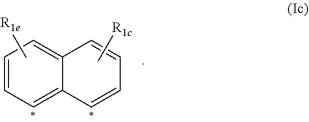

49. The composition of claim 48, wherein X is selected from the group consisting of a direct valence bond- and oxy (—O—).

50.-59. (canceled)

60. The composition of claim 1, wherein said compound of structure (I) in one wherein said core moiety has structure (Id), wherein X is methylene (—CH₂—) and the combination of A and X has structure (Id-1), where * designates the attachment point, and B is selected from the group consisting of —OH, —CH═CH₂ and —SH,

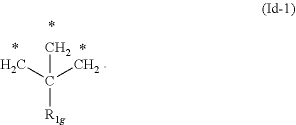

61. (canceled)

62. The composition of claim 1, wherein said compound of structure (I) in one wherein said core moiety has structure (Id), wherein X is a 1,4-phenyleneoxy (-Ph-O—) linking group, and the combination of A and X has structure (Id-3), where * designates the attachment point, and B is selected from the group consisting of —OH, —CH═CH₂ and —SH,

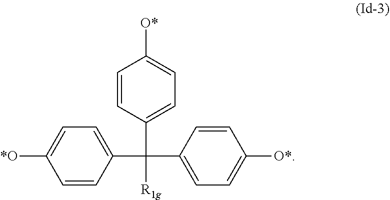

63.-78. (canceled)

79. The composition of claim 1 which is a PMMA affinity SAM composition, wherein in said compound (I),
at least one of R₁ₐ and R_{1b}, is a polar substituent selected from a C-1 to C-4 alkyloxy, a C-3 to C-6, methyl(carbonyloxyalkyl) (—CH₂—(C=O)—O-alkyl), a C-2 to C-5 carbonyloxyalkyl (—(C=O)—O-alkyl), a nitro (NO₂), NH₂, and CN;
at least one of R_{1c}, or R_{id} is a polar substituent selected from a C-1 to C-4 alkyloxy, a C-3 to C-6, methyl(carbonyloxyalkyl) (—CH₂—(C=O)—O-alkyl), a C-2 to C-5 carbonyloxyalkyl (—(C=O)—O-alkyl) and CN;
at least one of R₁ₑ, or R_{1f} is a polar substituent selected from a C-1 to C-4 alkyloxy, a C-3 to C-6, methyl(carbonyloxyalkyl) (—CH₂—(C=O)—O-alkyl), a C-2 to C-5 carbonyloxyalkyl (—(C=O)—O-alkyl) and CN;
R_{1g} is a polar substituent selected from a C-1 to C-4 alkyloxy, a C-3 to C-6, methyl(carbonyloxyalkyl) (—CH₂—(C=O)—O-alkyl), a C-2 to C-5 carbonyloxyalkyl (—(C=O)—O-alkyl) and CN, and further wherein if only one of either R₁ₐ and R_{1b}, or either R_{1c} and R_{1d}, or either R₁ₑ and R_{1f}, is such a polar substituent the other substituent is either H or a C-1 to C-4 alkyl.

80. The composition of claim 1 which is a PS affinity SAM composition, wherein in said compound (I),
at least one of R₁ₐ, or R_{1b} is a C-1 to C-4 alkyl, at least one of R_{1c}, or R_{1d} is a C-1 to C-4 alkyl, at least one of R₁ₑ, or R_{1f} is a C-1 to C-4 alkyl, R_{g} is a C-1 to C-4 alkyl, and further wherein if only one of either R₁ₐ and R_{1b}, or either R_{1c} and R_{1d}, or either R₁ₑ and R_{1f}, is a C-1 to C-4 alkyl, the other substituent is H.

81. The composition of claim 1 which is a neutral affinity SAM composition, wherein in said compound (I), R₁ₐ, R_{1b}, R_{1c}, R_{1d}, R₁ₑ, R_{1f}, and R_{1g} are H.

82. A process of coating with a PMMA affinity SAM composition, comprising the steps:
i) coating a substrate with the PMMA affinity SAM composition of claim 79,
ii) baking at a temperature ranging from about 150° C. to about 200° C.,
iii) rinsing with an organic spin casting solvent to form a self-assembled monolayer with an affinity to the PMMA block segments a block copolymer of polystyrene (PS) and methyl methacrylate (MMA) (PS-b-PMMA).

83. A process of coating with a PS affinity SAM composition, comprising the steps;
ia) coating a substrate with the PS affinity SAM composition of claim 80,
iia) baking at a temperature ranging from about 150° C. to about 200° C.,
iiia) rinsing with an organic spin casting solvent to form a self-assembled monolayer with an affinity to PS block segment of a block copolymer of polystyrene (PS) and methyl methacrylate (MMA)(PS-b-PMMA).

84. A process of coating with a Neutral affinity SAM composition, comprising the steps;
ib) coating a substrate with the Neutral affinity SAM composition of claim 81,
iib) baking at a temperature ranging from about 150° C. to about 200° C.,
iiib) rinsing with an organic spin casting solvent to form a self-assembled monolayer with a neutral affinity block segment of a block copolymer of polystyrene (PS) and methyl methacrylate (MMA) (PS-b-PMMA).

85. A process of selective deposition of a self-assembled monolayer, followed by directed self-assembly of a block copolymer comprising the steps:
ic) using lithography processing to form a chemoepitaxy array of metal and non-metal lines on a substrate,
iic) coating said substrate with a PMMA affinity brush polymer, or a PMMA affinity SAM composition wherein, said PMMA affinity SAM composition is a composition of claim 1, wherein said compound of structure (I) is one wherein,
A is a core moiety which is selected from structure (Ia), (Ib), (Ic) and (Id), to which is attached through X, a direct valence bond or a divalent linking group, m number of linear alkylene moieties of chain length n, wherein n ranges from 8 to 12 and where each said linear alkylene moiety has a terminal B reactive moiety, and further wherein * designates possible attachment point of said linear alkylene moieties in each structure, wherein structure (Ia), and (Ib), have 2 or 3 attachment points (m=2 or 3) which are on adjacent carbons, structure (Ic) has 2 attachment point (m=2), structure (Id), has three attachment point (m=3),
B is selected from —OH, —CH═CH₂, —O—(P=O)(OR)₂, —O—(P=O)(OR)Rs, —N₃, and —SH, wherein R and
Rs are independently selected from a C-1 to C-8 alkyl
R₁ₐ and R_{1b} are independently selected from H a C-1 to C-4 alkyl, a C-1 to C-4 alkyloxy, a C-3 to C-6 methyl(carbonyloxyalkyl) (—CH₂—(C=O)—O-alkyl), a C-2 to C-5 carbonyloxyalkyl (—(C=O)—O-alkyl), nitro (NO₂), NH₂, and CN,
R_{1c}, R_{1d}, R₁ₑ, R_{1f} and R_{1g} are independently selected from H a C-1 to C-4 alkyl, a C-1 to C-4 alkyloxy, C-2 to C-5 carbonyloxyalkyl (—(C=O)—O-alkyl), a C-3 to C-6 methylcarbonyloxyalkyl (—CH₂—(C=O)—O-alkyl), and CN; and further wherein,
a least one of R₁ₐ and R_{1b}, is a polar substituent selected from a C-1 to C-4 alkyloxy, a C-3 to C-6 methyl(carbonyloxyalkyl) (—CH₂—(C=O)—O-alkyl), a C-2 to C-5 carbonyloxyalkyl (—(C=O)—O-alkyl), a Nitro (NO₂), NH₂, and CN;
at least one of R_{1c}, or R_{id} is a polar substituent selected from a C-1 to C-4 alkyloxy, a C-3 to C-6 methyl(carbonyloxyalkyl) (—CH₂—(C=O)—O-alkyl), a C-2 to C-5 carbonyloxyalkyl (—(C=O)—O-alkyl) and CN;
at least one of R₁ₑ, or R_{1f} is a polar substituent selected a C-1 to C-4 alkyloxy, a C-3 to C-6 methyl(carbonyloxyalkyl) (—CH₂—(C=O)—O-alkyl), a C-2 to C-5 carbonyloxyalkyl (—(C=O)—O-alkyl) and CN;
R_{1g} is a is a polar substituent selected from a C-1 to C-4 alkyloxy, a C-3 to C-6, methyl(carbonyloxyalkyl) (—CH₂—(C=O)—O-alkyl), a C-2 to C-5 carbonyloxyalkyl (—(C=O)—O-alkyl) and CN, and further wherein if only one of either R₁ₐ and R_{1b}, or either R_{1c} and R_{1d}, or either R₁ₑ and
R_{1f}, is such a polar substituent the other substituent is H or a C-1 to C-4 alkyl,
iiic) baking at a temperature ranging from 150° C. to 200° C.,
ivc) rinsing with an organic spin casting solvent the produce a substrate in which only the metal lines have an attached PMMA affinity brush, or a PMMA affinity self-assembled monolayer,
ve) coating with the Neutral affinity SAM composition of claim 81,
vic) baking at a temperature ranging from about 150° C. to about 200° C.,
viic) rising with an organic spin casting solvent to produce a substrate in which the metal lines still have an attached PMMA brush, but also have a Neutral affinity self-assembled monolayer attached to the non-metal lines,
viiic) coating the substrate formed in step viic) with a block copolymer solution,
vivc) annealing the block copolymer coating to form directed self-assembled block copolymer L/S pattern.

86. The process of claim 85, wherein the PMMA affinity SAM composition is a PMMA affinity SAM composition wherein in said compound (I),
at least one of R₁ₐ and R_{1b}, is a polar substituent selected from a C-1 to C-4 alkyloxy, a C-3 to C-6, methyl(carbonyloxyalkyl) (—CH₂—(C=O)—O-alkyl), a C-2 to C-5 carbonyloxyalkyl (—(C=O)—O-alkyl), a nitro (NO₂), NH₂, and CN;
at least one of R_{1c}, or R_{id} is a polar substituent selected from a C-1 to C-4 alkyloxy, a C-3 to C-6, methyl(carbonyloxyalkyl) (—CH₂—(C=O)—O-alkyl), a C-2 to C-5 carbonyloxyalkyl (—(C=O)—O-alkyl) and CN;
at least one of R₁ₑ, or R_{1f} is a polar substituent selected from a C-1 to C-4 alkyloxy, a C-3 to C-6, methyl(carbonyloxyalkyl) (—CH₂—(C=O)—O-alkyl), a C-2 to C-5 carbonyloxyalkyl (—(C=O)—O-alkyl) and CN;
R_{1g} is a polar substituent selected from a C-1 to C-4 alkyloxy, a C-3 to C-6, methyl(carbonyloxyalkyl) (—CH₂—(C=O)—O-alkyl), a C-2 to C-5 carbonyloxyalkyl (—(C=O)—O-alkyl) and CN, and further wherein if only one of either R₁ₐ and R_{1b}, or either R_{1c} and R_{1d}, or either R₁ₑ and R_{1f}, is such a polar substituent the other substituent is either H or a C-1 to C-4 alkyl.

87. A process of selective removal of a self-assembled monolayer and directed self-assembly of a polymer comprising the steps:
id) using lithography processing to form a chemoepitaxy array of metal and non-metal lines on a substrate,
iid) coating with a composition of the PS affinity SAM composition of claim 80,
iiid) baking at a temperature ranging from about 150° C. to about 200° C.,
ivd) rinsing with an organic spin casting solvent to produce a substrate in which the whole substrate has an attached PS affinity self-assembled monolayer, on both the metal and non-metal lines,
vd) treating with a removal solution, to selectively remove the PS affinity self-assembled monolayer, wherein said removal solution is selected from the group consisting of
a removal solution with a pH from about 3 to about 5 comprising, water, glycine, hydrogen peroxide solutions and a secondary acid component selected from a dicarboxylic acid, a tricarboxylic acid or an aryl sulfonic acid substituted with an alkyl,
a removal solution with a pH from about 3 to about 5 consisting of an alcoholic glycolic spin casting solvent, water, and an acid component selected from tricarboxylic acid, having a pKₐ from about 2.9 to about 5, a dicarboxylic acid having a pKₐ from about 3 to about 5 or an alkyl substituted aryl sulfonic acid,
a removal solution consisting of an alcoholic glycolic spin casting solvent, and a dithiol and
a removal solution consisting of an alcoholic glycolic spin casting solvent, and an amine,
vid) optionally baking at a temperature ranging from about 100° C. to about 200° C.,
viid) rinsing with an organic spin casting solvent to produce a substrate in which on the whole substrate only has PS affinity self-assembled monolayer on the non-metal lines,
viiid) coating with the composition of a Neutral affinity SAM composition of claim 81,
ixd) baking at a temperature ranging from about 150° C. to about 200° C.,
xd) rinsing with an organic spin casting solvent to produce a substrate in which the metal lines have an attached Neutral affinity self-assembled monolayer, and the non-metal lines have an attached PS affinity self-assembled monolayer,
xid) coating the substrate formed in step ixd) with a solution of a block copolymer which has polar and non-polar block segments,
xiid) annealing the block copolymer coating to form directed self-assembled block copolymer L/S pattern.

88. A process of coating with a PMMA affinity SAM composition on a chemoepitaxy array of metal and non-metal lines and selectively removing deposited SAM on the metal lines, comprising the steps:
ie) using lithography processing to form a chemoepitaxy array of metal and non-metal lines on a substrate,
iie) coating a substrate with the PMMA affinity SAM composition of claim 79,
iiie) baking at a temperature ranging from about 150° C. to about 200° C.,
ive) rinsing with an organic spin casting solvent to form a PMMA affinity self-assembled monolayer on both the metal lines and non-metal lines,
ve) treating with a removal solution, to selectively remove the PMMA affinity self-assembled monolayer from the metal lines, wherein said removal solution is selected from the group consisting of
a removal solution with a pH from about 3 to about 5 comprising, water, glycine, hydrogen peroxide solutions and a secondary acid component selected from a dicarboxylic acid, a tricarboxylic acid or an aryl sulfonic acid substituted with an alkyl,
a removal solution with a pH from about 3 to about 5 consisting of an alcoholic glycolic spin casting solvent, water, and an acid component selected from tricarboxylic acid, having a pKₐ from about 2.9 to about 5, a dicarboxylic acid having a pKₐ from about 3 to about 5 or an alkyl substituted aryl sulfonic acid,
a removal solution consisting of an alcoholic glycolic spin casting solvent, and a dithiol and
a removal solution consisting of an alcoholic glycolic spin casting solvent, and an amine,
vie) optionally baking at a temperature ranging from about 100° C. to about 200° C.,
viie) treating with an organic spin casting solvent to remove the cleaved SAM producing a substrate in which only the non-metal lines have attached a PMMA affinity self-assembled monolayer.

89. A process of coating with a PS affinity SAM composition on a chemoepitaxy array of metal and non-metal lines and selectively removing deposited SAM on the metal lines, comprising the steps:
if) using lithography processing to form a chemoepitaxy array of metal and non-metal lines on a substrate,
iif) coating a substrate with the PS affinity SAM composition of claim 80,
iiif) baking at a temperature ranging from about 150° C. to about 200° C.,
ivf) rinsing with an organic spin casting solvent to form a PS affinity self-assembled monolayer on both the metal lines and non-metal lines,
vf) treating with a removal solution to selectively remove the PS affinity self-assembled monolayer from the metal lines, wherein said removal solution is selected from the group consisting of
a removal solution with a pH from about 3 to about 5 comprising, water, glycine, hydrogen peroxide solutions and a secondary acid component selected from a dicarboxylic acid, a tricarboxylic acid or an aryl sulfonic acid substituted with an alkyl,
a removal solution with a pH from about 3 to about 5 consisting of an alcoholic glycolic spin casting solvent, water, and an acid component selected from tricarboxylic acid, having a pKₐ from about 2.9 to about 5, a dicarboxylic acid having a pKₐ from about 3 to about 5 or an alkyl substituted aryl sulfonic acid,
a removal solution consisting of an alcoholic glycolic spin casting solvent, and a dithiol and
a removal solution consisting of an alcoholic glycolic spin casting solvent, and an amine,
vif) optionally baking at a temperature ranging from about 100° C. to about 200° C.,
viif) treating with organic spin casting solvent to remove the cleaved PS affinity self-assembled monolayer from the metal lines producing a substrate in which only the non-metal lines have attached a PS affinity self-assembled monolayer.

90. A process of coating with a Neutral affinity SAM composition on a chemoepitaxy array of metal and non-metal lines and selectively removing deposited SAM on the metal lines, comprising the steps:
ig) using lithography processing to form a chemoepitaxy array of metal and non-metal lines on a substrate,
iig) coating a substrate with the Neutral affinity SAM composition of claim 81,
iiig) baking at a temperature ranging from about 150° C. to about 200° C.,
ivg) rinsing with an organic spin casting solvent to form a Neutral affinity self-assembled monolayer on both the metal lines and non-metal lines,
vg) treating with a removal solution to selectively remove the Neutral affinity self-assembled monolayer from the metal lines, wherein said removal solution is selected from the group consisting of
a removal solution with a pH from about 3 to about 5 comprising water, glycine, hydrogen peroxide solutions and a secondary acid component selected from a dicarboxylic acid, a tricarboxylic acid or an aryl sulfonic acid substituted with an alkyl,
a removal solution with a pH from about 3 to about 5 consisting of an alcoholic glycolic spin casting solvent, water, and an acid component selected from tricarboxylic acid, having a pKₐ from about 2.9 to about 5, a dicarboxylic acid having a pKₐ from about 3 to about 5 or an alkyl substituted aryl sulfonic acid,
a removal solution consisting of an alcoholic glycolic spin casting solvent, and a dithiol and
a removal solution consisting of an alcoholic glycolic spin casting solvent, and an amine,
vig) optionally baking at a temperature ranging from about 100° C. to about 200° C.,
viig) treating with an organic spin casting solvent to remove the cleaved Neutral affinity self-assembled monolayer from the metal lines producing a substrate in which only the non-metal lines have attached a Neutral affinity self-assembled monolayer.

91.-101. (canceled)
